(19) United States
(12) Patent Application Publication
(10) Pub. No.: US 2019/0046576 A1
(43) Pub. Date: Feb. 14, 2019

(54) ADIPOSE TISSUE DERIVED MESENCHYMAL STROMAL CELL CONDITIONED MEDIA AND METHODS OF MAKING AND USING THE SAME

(71) Applicant: Cell Care Therapeutics, Monrovia, CA (US)

(72) Inventors: Raja Shekhar Gangaraju, Colliersville, TN (US); Nicolas Miroslav Jotterand Sohl, Long Beach, CA (US); Veronique Hedwige Jotterand, Long Beach, CA (US); Mickey Pentecost, West Hollywood, CA (US)

(73) Assignee: Cell Care Therapeutics, Monrovia, CA (US)

(21) Appl. No.: 16/076,511

(22) PCT Filed: Feb. 13, 2017
(86) PCT No.: PCT/US2017/017718

§ 371 Date: Aug. 8, 2018

Related U.S. Application Data
(60) Provisional application No. 62/294,489, filed on Feb. 12, 2016.
(60) Provisional application No. 62/414,285, filed on Oct. 28, 2016.

(51) Int. Cl. A61K 35/28; A61K 47/10; A61K 9/19; A61K 9/00; A61K 47/12; A61K 47/14; A61K 47/18; A61K 47/22; A61K 47/26; A61P 27/02; A61K 9/08

(52) U.S. Cl. CPC A61K 35/28; A61K 47/10; A61K 9/19; A61K 9/0019; A61K 9/0048; A61K 9/08; A61K 47/14; A61K 47/183; A61K 47/22; A61K 47/26; A61P 27/02; A61K 47/12

(57) ABSTRACT

Provided herein are lyophilized compositions containing the secretome of cultured adipose cells, pharmaceutical compositions and that additionally contain a sustained release drug delivery matrix, as well as methods of making and using such compositions.

### RELATED APPLICATIONS

This application claims priority to U.S. Provisional Application No. 62/294,489, filed Feb. 12, 2016 and U.S. Provisional Application No. 62/414,285, filed Oct. 28, 2016, each of which is herein incorporated by reference in its entirety.

### FIELD OF INVENTION

This invention relates generally to compositions comprising stem cell secretions derived from conditioned media as well as methods of making and using the same.

### BACKGROUND OF THE INVENTION

Regenerative medicine is an area of medicine that is concerned with the replacement or regeneration of human cells, tissues, or organs, in order to restore or establish normal functions. For example, stem cell therapies can be utilized in order to treat, prevent, or cure a variety of diseases and disorders.

Stem cells are cells that have the ability to divide without limit and that, under certain specific conditions, can differentiate into a variety of different cell types. Totipotent stem cells are stem cells that have the potential to generate all of the cells and tissues that make up an embryo. Pluripotent stem cells are stem cells that give rise to cells of the mesoderm, endoderm, and ectoderm. Multipotent stem cells are stem cells that have the ability to differentiate into two or more cell types, whereas unipotent stem cells are stem cells that differentiate into only one cell type.

However, it is difficult to produce and store live stem cells therapies on a clinically relevant scale. (See Trainor et al., Nature Biotechnology 32(1) (2014). Moreover, the therapeutic potency and regenerative capacity of such therapies is often variable and the cells can die before or during transplantation. (See Newell, Seminars in Immunopathology 33(2):91 (2011)). Implanted stem cells are also susceptible to host immune system attack, and it is often difficult to assess potency and/or control “dosing”. Thus, there is a need in the art for additional regenerative therapies that can overcome the cost, storage, and manufacturing quality control limitations that are currently associated with cell-based regenerative medicine therapies.

Moreover, there is also large, unmet need in the art for ocular therapies that can target the back of the eye and deliver a therapeutic payload long-term to difficult-to-reach sensory tissue in the retina that have degenerated due to inflammation secondary to trauma, acute inflammation, age and/or oxidative stress.

### SUMMARY OF INVENTION

Retinal neuroprotection from inflammation secondary to acute or chronic metabolic disease remains a major area of unmet medical need for patients with back of the eye diseases and is not currently achieved with the current standard of care utilizing anti-VEGF therapies. Activation of immune cells, including retinal microglia is a common feature of degenerative retinal diseases, including diabetic retinopathy and dry AMD, and may be responsible for initiating and propagating chronic neuroinflammation and neurodegenerative processes leading to gliosis, hemorrhaging, geographic atrophy, breakdown of the retinal pigmented epithelium (RPE), and vascular leakage leading to loss of visual function. (See Madeira et al., Mediators of Inflammation 2015:673090 (2015)).

Adipose stromal cells (ASC or ADSC), through paracrine signaling, arrest apoptosis and protect against glial scarring and degeneration of endothelial tight junctions by reducing microglia activation and t-cell proliferation through the secretion of soluble and membrane bound chemokines, cytokines, growth factors, angiogenic factors, and miRNA. (See Cai et al., Stem Cells 25(12):3234-3243 (2007)).

While one of the major mechanisms of immunomodulation by mesenchymal stem cells (MSC) is the regulation of T and NK cells, more recently MSC have also been shown to polarize macrophages from the classic proinflammatory M1 phenotype, toward the anti-inflammatory M2 phenotype. (See Kim et al., Exp Hematol 37(12): 1445-1453 (2009).

Harvesting and processing ASC secretions, released by the cells for paracrine signaling, offers the possibility of developing cost effective, shelf-stable, and cell-free regenerative therapies that represent an appealing treatment alternative to direct ASC transplantation.

Provided herein are compositions of processed lyophilized adipose stromal cell secretions that are shelf stable, easily reconstituted, and intravitreally injected for ophthalmic use. These compositions have been tested in animal studies and recapitulate the regenerative neurovascular protective effect of ASC delivered to the retina.

Also provided herein are scalable cGMP compliant manufacturing processes that permit the enhancement of ASC paracrine activity and potency of secretions by pre-activating the cells under inflammatory conditions that mirror the inflammatory in vivo retinal milieu. It has been demonstrated that the combination of cytokines, including TNF-α and IFN-γ, have synergistic effects on the expression of key regenerative proteins. MSC pre-stimulation of the cells prior to collection of secretions increases the regenerative and neuroprotective capacity of the therapeutic, indicating the importance of integrating second order cytokine pre-treatment combinations into the manufacturing process.

Provided herein are lyophilized compositions containing a concentrated, cell-free secretome of cultured adipose cells, wherein the adipose cells comprise at least one adipose stem cell (ASC) and wherein at least 90% (i.e., at least 90, 91, 92, 93, 94, 95, 96, 97, 98, 99, or 100%) of the cultured adipose cells express not only mesenchymal markers but also at least one pericyte marker; and an effective amount of a lyophilizing agent. By way of non-limiting example, the pericyte marker may be selected from the group consisting of CD140b, CD146, and Neural/glial antigen 2 (NG2). These cells may also be positive for classical MSC markers including, for example, CD73, CD90, CD105, and/or negative for classical leukocyte and endothelial markers such as CD45, CD14, CD19, HLA-DR and CD31. (See FIG. 1). The expression of one or more of pericyte markers by at least 90% of the ASCs may influence the therapeutic efficacy of the compositions containing the concentrated, cell-free secretome of the adipose cells. By way of non-limiting example, the expression of the pericyte markers may increase the potency of any of the compositions described herein.

Examples of suitable lyophilizing agents include, for example, Tris-EDTA and sucrose. In one embodiment, the composition additionally includes an effective amount of a buffer for filtration. For example, Tris-EDTA (e.g., about 25 mM Tris and about 1 mM EDTA) can be selected as the buffer used to filter, which influences the lyophilization cycle, and sucrose can be selected as the lyophilization agent that acts as a protein stabilizer to protect the product during the freezing cycle. Any other lyophilizing agents commonly used in the art can also be utilized. Determination of the appropriate lyophilization agent as well as the effective amount of other lyophilizing agents is within the routine level of skill in the art.

Adipose cells can be obtained by any method(s) commonly used in the art. For example, the adipose cells may be obtained from a male or female subject following a liposuction procedure.

Any of the lyophilized compositions described herein are shelf-stable at a temperature between about 20 and 35° C. (i.e., 20, 21, 22, 23, 24, 25, 26, 27, 28, 29, 30, 31, 32, 33, 34, or 35° C.) for a period of at least 2, 3, 4, 5, 6, 7, 8, 9, 10, 11, or 12 weeks. (See, e.g., FIG. 4C). In some embodiments, the compositions are shelf-stable for a period of at least 3 months (i.e., at least 3, 4, 5, 6, 7, 8, 9, 10, 11, 12, or more months).

Preferably, these lyophilized compositions are non-immunogenic. (See, e.g., FIGS. 13A, 13B, and 14).

Also provided are pharmaceutical compositions containing an effective amount of any of the lyophilized compositions described herein and a sustained release drug delivery matrix. For example, the sustained release drug delivery matrix is biodegradable and/or biocompatible. In various embodiments, the sustained release drug delivery matrix is selected from the group consisting of a gel, a paste-like composition, a semi-solid composition, and a microparticulate composition.

These gels, paste-like compositions, and semi-solid compositions can be mechanically formed through macroscopic processing. Examples of the manufacture of sustained release drug delivery matrices are provided in U.S. 20140356435, U.S. 20130136775, U.S. Application No. 62/060,642, and PCT/US15/54249, which are herein incorporated by reference in their entireties.

Preferably, the sustained release drug delivery matrix does not cause any chemical or biological changes to the lyophilized composition. For example, the sustained release drug delivery matrix may be hydrophobic or hydrophilic in nature.

In various embodiments, the sustained release drug delivery matrix is a hydrophobic matrix. The hydrophobic matrix may include one or more hydrophobic excipients selected from the group consisting of magnesium stearate, magnesium palmitate, fatty acid salts, cetyl palmitate, fatty acid salts, plant oils, fatty acid esters, tocopherols, and combinations thereof. In one example, the hydrophobic matrix contains magnesium stearate and tocopherol.

In some embodiments, the hydrophobic matrix contains at least a hydrophobic solid component and a hydrophobic liquid component. Examples of suitable hydrophobic solid components include, but are not limited to, waxes, fruit wax, carnauba wax, bees wax, waxy alcohols, plant waxes, soybean waxes, synthetic waxes, triglycerides, lipids, long-chain fatty acids (i.e., magnesium stearate) and their salts, magnesium palmitate, esters of long-chain fatty acids, long-chain alcohols (i.e., cetyl palmitate or cetyl alcohol), waxy alcohols, oxethylated plant oils, and oxethylated fatty alcohols. Moreover, examples of suitable liquid hydrophobic components include, but are not limited to, plant oils, castor oil, jojoba oil, soybean oil, silicon oils, paraffin oils, and mineral oils, cremophor, oxethylated plant oils, oxethylated fatty alcohols, tocopherols, lipids, and phospholipids.

The effective amount of the lyophilized composition can be between about 0.01 and about 50% (w/w) (i.e., 0.01, 0.02, 0.03, 0.04, 0.05, 0.06. 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (w/w)). In some embodiments, the amount of the lyophilized composition in the pharmaceutical composition will be lower (i.e., 20, 15, 10, 5, 1, 0.5, 0.1, 0.05% (w/w)).

Those skilled in the art will recognize that in any of the pharmaceutical compositions, the amount of the active ingredient(s) (i.e., the secretome) may be between 0.01% and 10% (i.e., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2.0, 2.5, 3.0, 3.5, 4.0, 4.5, 5.0, 5.5, 6.0, 6.5, 7.0, 7.5, 8.0, 8.5, 9.0, 9.5, or 10%).

The lyophilized composition can be dispersed in the hydrophobic matrix in particulate form or in a dissolved state.

The pharmaceutical compositions may additionally contain at least one excipient selected from the group consisting of monosaccharides, disaccharides, oligosaccharides, polysaccharides, hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, or mixtures thereof.

The pharmaceutical compositions described herein may also contain one or more anti-caking agents. For example, the anti-caking agent is a compound selected from the group consisting of magnesium stearate, magnesium palmitate and other similar compounds.

The pharmaceutical compositions described herein may also contain at least one polymer. The polymer may be selected from the group consisting of polyvinyl alcohol (PVA), polyvinylpyrrolidone (PVP), polyethylene glycol (PEG), gelatin, collagen, alginate, starch, cellulose, chitosan, carboxymethylcellulose, cellulose derivatives, pectin, gum arabic, carrageenan, hyaluronic acid, albumin, fibrin, fibrinogen, synthetic polyelectrolytes, polyethylenimine, acacia gum, xanthan gum, agar agar, polyvinylalcohol, borax, polyacrylic acids, protaminsulfate and casein.

Any of the compositions described herein release therapeutically effective amounts of regenerative and anti-inflammatory factors from the secretome of the adipose cells for a period of up to 6 months (i.e. , 1, 2, 3, 4, 5, or 6 months). Non-limiting examples of regenerative or anti-inflammatory factors can include proteins (e.g., cytokines, chemokines, growth factors, enzymes), nucleic acids (e.g., microRNA (miRNA)), lipids (e.g., phospholipids), polysaccharides, and/or combinations thereof, either bound within or on the surface of extracellular vesicles (e.g., exosomes) or separate from extracellular vesicles. Those skilled in the art will recognize that these regenerative or anti-inflammatory factors may act to stimulate tissue regeneration, vascular (e.g., neurovascular) repair, or both tissue regeneration and vascular (e.g., neurovascular) repair.

To generate any of the compositions described herein, the at least one ASC may be cultured under conditions that increase the expression of the one or more regenerative or anti-inflammatory factors.

The total protein included in any of the compositions described herein may be between 0.01 mg/ml and 1.5 mg/ml (e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, or 1.5 mg/ml). By way of non-limiting example, the secretome of ASCs cultured according to any of the methods disclosed herein may include one or more of the following proteins: Tumor necrosis factor-inducible gene 6 protein (also known as TSG-6) (Gene: TNFAIP6; UniProtKB ID: P98066), Metalloproteinase inhibitor 1 (TIMP1; UniProtKB ID: P01033), Metalloproteinase inhibitor 2 (TIMP2; UniProtKB ID: P16035), SPARC (UniProtKB ID: P09486), Insulin-like growth factor-binding protein 3 (IGFBP3; UniProtKB ID: P17936), Insulin-like growth factor-binding protein 4 (IGFBP4; UniProtKB ID: P22692), Insulin-like growth factor-binding protein 6 (IGFBP6; UniProtKB ID: P24592), Insulin-like growth factor-binding protein 5 (IGFBP5; UniProtKB ID: P24593), Insulin-like growth factor-binding protein 7 (IGFBP7; UniProtKB ID: Q16270), Vascular endothelial growth factor C (VEGFC; UniProtKB ID: P49767), Plasminogen activator inhibitor 2 (SERPINB2; UniProtKB ID: P05120), Serpin B6 (SERPINB6; UniProtKB ID: P35237), Antithrombin-III (SERPINC1; UniProtKB ID: P01008), Plasminogen activator inhibitor 1 A.K.A PAI1 (SERPINE1; UniProtKB ID: P05121), Glia-derived nexin (SERPINE2; UniProtKB ID: P07093), Pigment epithelium-derived factor (also known as PEDF (SERPINF1; UniProtKB ID: P36955), Plasma protease C1 inhibitor (SERPING1; UniProtKB ID: P05155), Serpin H1 (SERPINH1; UniProtKB ID: P50454), CD81 antigen (CD81; UniProtKB ID: P60033), CD63 antigen (CD63; UniProtKB ID: P08962), Tissue factor pathway inhibitor 2 (TFPI2; UniProtKB ID: P48307), 72 kDa type IV collagenase (MMP2; UniProtKB ID: P08253), Interstitial collagenase (MMP1; UniProtKB ID: P03956), Matrix metalloproteinase-14 (MMP14; UniProtKB ID: P50281) and Galectin-1 (LGALS1; UniProtKB ID: P09382).

FIG. 7E shows 100 abundant proteins that are preserved in processed ASC-CM and CC-101 (in both histidine buffer and Tris/EDTA).

Any of the culture methods described herein (e.g., culturing in the presence of exogenously added amounts of IFNγ and TNFα) may result in a two or more fold change (i.e., 2, 3, 4, 5, 6, 7, 8, 9, 10 or more) in expression of one or more of the following cytokines and chemokines: Growth-regulated alpha protein (CXCL1; UniProtKB ID: P09341), interleukin-6 (IL6; UniProtKB ID: P05231), interleukin-8 (IL-8, CXCL8; UniProtKB ID: P10145), C-C motif chemokine 2 (CCL2; UniProtKB ID: P13500), C-C motif chemokine 8 (CCL8; UniProtKB ID: P80075), C-C motif chemokine 5 (CCL5; UniProtKB ID: P13501), C-X-C motif chemokine 10 (CXCL10; UniProtKB ID: P02778), or Tumor necrosis factor receptor superfamily member 11B (TNFRSF11B; UniProtKB ID: 000300). (See FIGS. 7A and 7B).

GRO/CXCL1 has been shown to mediate the stimulatory effects of ASCs on endothelial cells. (See Zhang et al., Nature Communications 7.11674 (2016)).

MSC conditioned medium inhibits EAE-derived CD4 T cell activation by suppressing STAT3 phosphorylation via MSC-derived CCL2, and further analysis demonstrates that the effect is dependent on MSC-driven matrix metalloproteinase proteolytic processing of CCL2 to an antagonistic derivative. (See Rafei et al., J. Immunol. 182:5994-6002 (2009)).

ASCs cultured according to any of the methods disclosed herein may express one or more extracellular vesicles (EVs). For example, the extracellular vesicles may be an exosome, microvesicle, membrane particle, membrane vesicle, exosome-like vesicle, ectosome-like vesicle, ectosome or exovesicle. Extracellular vesicles likely play a role in intercellular communication by acting as vehicles between a donor and recipient cell through paracrine mechanisms.

Exosomes usually express tetraspanins, integrins, MHC Class I and/or Class II antigens, CD antigens and cell-adhesion molecules on their surfaces. Exosomes contain a variety of clathrin, GTPases, cytoskeletal proteins, chaperones, and metabolic enzymes (not including lysosomal, mitochondrial ER proteins as to exclude a cytoplasm profile). They also contain mRNA splicing and translation factors. The pre (ASC-CM) and post-lyophilized (CC-101) compositions described herein contain numerous examples of proteins compiled in ExoCarta, an online database of putative exosome constituents (FIG. 7C). Moreover, functional enrichment analysis of the ASC-CM/CC-101 proteome indicates a statistically significant over-representation of proteins in the gene ontology class “extracellular exosome” (GO: 0070062) (FIG. 7D).

Any of the compositions described herein may contain between 1×10⁸ and 9×10¹¹ (i.e., 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹¹, 2×10¹¹, 3×10¹¹, 4×10¹¹, 5×10¹¹, 6×10¹¹, 7×10¹¹, 8×10¹¹, 9×10¹¹) extracellular vesicles between 30 nm and 1000 nm (e.g., 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nm), or between 30 and 500 nm, or between 30 and 300 nm, as determined by tunable resistance pulse sensing. Extracellular vesicles described herein may contain one or more classical exosomal markers. By way of non-limiting example, the classical exosomal marker may be one or more tetraspanins optionally selected from CD53, CD63, CD9, CD81, CD82, and/or CD37. Alternatively (or additionally), the exosomal marker may be 14-3-3.

MicroRNAs (miRNAs) are a family of conserved, short (approximately 22 nucleotide), single stranded RNA molecules that are found in plants, animals, and some virus. miRNAs function to regulate posttranscriptional gene expression levels. miRNAs can be found both within cells and in extracellular environments (such as biological fluids and cell culture media). MicroRNAs are putative cargo of extracellular vesicles such as exosomes. MicroRNAs may play a role in a variety of processes including, for example, development, differentiation, homeostasis, metabolism, growth, proliferation, and apoptosis. (See Landskroner-Eiger et al., Cold Spring Harb Perspect Med 3:a06643 (2013)).

By way of non-limiting example, the secretome of ASCs cultured according to any of the methods disclosed herein may alternatively or additionally include one or more precursor or mature miRNAs selected from hsa-miR-221/222, hsa-miR-199, hsa-miR-22, hsa-miR-16, and/or hsa-miR-26.

miR-221/222 has been shown to target pro-angiogenic c-KIT. Overexpression of this miRNA reduces endothelial tube formation, migration, and wound healing in response to Stem Cell Factor (SCF). (See Landskroner-Eiger et al., at Table 1).

miR-199 has been implicated in a wide variety of cellular and developmental mechanisms. These include cancer development and progression; protection of cardiomyocytes; and/or skeletal muscle formation. miR-199 may also regulate angiogenic processes. (See Dai et al., Int J Clin Pathol. 8(5):4735-4744 (2015); He et al., PloS One 8(2):e56647 (2013)).

miR-22 can function as a tumor suppressor. Known targets of miR-22 include histone deacetylase 4 (HDAC4) and Myc Binding Protein (MYCBP). miR-22 can inhibit in vitro angiogenesis by targeting AKT3. (See Zheng et al., Cell Physiol Biochem 34(5):1547-1555 (2014)).

miR-16 may be involved with cellular differentiation. miR-16 can target VEGF mRNA and suppress angiogenesis. (See Lee et al., PloS One 8(12):e84256 (2013)).

miR-26 expression is induced in response to hypoxia and upregulated during smooth muscle cell (SMC) differentiation and neurogenesis. miR-26 expression is also down-regulated in certain malignant tumors (e.g., hepatocellular carcinoma, nasopharyngeal carcinoma, lung cancer, and breast cancer) and overexpressed in some cancers (e.g., high-grade glioma, cholangiocarcinoma, pituitary tumors, and bladder cancer). miR-26 also regulates angiogenesis through various targets. (See Chai et al., PloS One 8(10):e77957 (2013); Icli et al., Circ Res 113(11):1231-1241 (2013)).

Also provided are dosage forms containing any of the pharmaceutical compositions described herein, wherein the dosage form has a size and shape suitable for injection into a human or mammalian eye. For example, the pharmaceutical composition may be injected into the eye as a suspension through a 29 gauge needle.

In one embodiment, the pharmaceutical compositions described herein may be micronized prior to administration. In general, the term “micronized” is defined as having been through the process of reducing the average diameter of a solid material's particles. Any suitable micronization technique may be used in order to achieve the desired result. In another embodiment, the sustained release drug delivery composition comprising the lyophilized composition described herein is in a microparticulate form. Any other suitable dosage form and/or mode of administration may also be used.

Also provided are methods of treating ophthalmic disorders in a patient by administering an effective amount of any of the lyophilized compositions and/or pharmaceutical compositions described herein to a patient. Further provided are the lyophilized compositions and/or pharmaceutical compositions described herein for use in treating ophthalmic disorders in a patient. The lyophilized compositions and/or pharmaceutical compositions are for administration to the patient in an effective amount. For example, the ophthalmic disorder is an inflammatory and/or degenerative disease effecting vascular and/or neurological function of the retina such as the treatment of wet and dry AMD, diabetic retinopathy, retinopathy of prematurity, punctate inner choroidopathy, retinal branch vein occlusion, iritis, uveitis, endophthalmitis, optic neuropathies, glaucoma, Stargardt's Disease, retinal detachment, Retinitis Pigmentosa, Juvenile retinoschisis, senile retinoschisis, limbal stem cell deficiency, corneal surface diseases, traumatic injuries of the cornea, traumatic brain injuries, traumatic ocular injuries, traumatic injuries of the brain effecting vision and/or the retina.

In one embodiment, an effective amount of any of the pharmaceutical compositions described herein is administered to a patient in order to treat an inflammatory and/or degenerative ophthalmic disease effecting vascular and/or neurological function selected from wet and dry AMD, diabetic retinopathy, retinopathy of prematurity, punctate inner choroidopathy, retinal branch vein occlusion, iritis, uveitis, endophthalmitis, optic neuropathies, glaucoma, Stargardt's Disease, retinal detachment, Retinitis Pigmentosa, Juvenile retinoschisis, senile retinoschisis, limbal stem cell deficiency, corneal surface diseases, traumatic ocular injuries including injury to the cornea, traumatic brain injuries, traumatic injuries of the brain effecting vision and/or the retina.

In a different embodiment, an effective amount of any of the lyophilized compositions described herein is administered to a patient in order to treat an inflammatory and/or degenerative ophthalmic disease effecting vascular and/or neurological function is selected from wet and dry AMD, diabetic retinopathy, retinopathy of prematurity, punctate inner choroidopathy, retinal branch vein occlusion, iritis, uveitis, optic neuritis, glaucoma, Stargardt's Disease, retinal detachment, Retinitis Pigmentosa, Juvenile retinoschisis, senile retinoschisis, limbal stem cell deficiency, corneal surface diseases, traumatic ocular injuries including the injury to the cornea, traumatic brain injuries, traumatic injuries of the brain effecting vision and/or the retina.

In any of the methods described herein, the pharmaceutical composition and/or lyophilized composition is administered at least every 2-6 months (i.e., at least every 2, 3, 4, 5, or 6 months).

The pharmaceutical composition and/or lyophilized composition can be administered topically to the eye of the patient or by injection (e.g., by intraocular injection). For example, the pharmaceutical composition or lyophilized composition is injected into the vitreous chamber of the eye, injected sub-conjunctivally, injected sub-tenon (see, e.g., Weiss et al., Neural Regen Res 10(6):982-988 (2015), injected retrobulbar, and/or injected intra-retinally, for example through a 29 gauge needle.

Those skilled in the art will recognize that the anti-inflammatory and regenerative factors released from the pharmaceutical composition or the lyophilized composition can exert a biological function in the patient. By way of non-limiting example, these regenerative factors can protect and/or stimulate regrowth of pericytes, endothelial cells, ganglion cells and astrocytes and/or decrease glial activation. Alternatively (or additionally), the regenerative factors can decrease vascular permeability, decrease abnormal vascular growth, improve retinal thickness, reduce damage to neurovascular tissue, reduce gliosis, improve or protect retinal function, improve or protect neurological function, improve or protect vision, or any combination thereof.

In one embodiment, the pharmaceutical contains 0.5-1 ml of the lyophilized composition (e.g., 0.5, 0.6, 0.7. 0.8, 0.9, or 1 ml) and the sustained release drug delivery matrix. For example, the pharmaceutical composition is micronized into a suspension for intravitreal injection.

Also provided are methods of making the lyophilized compositions described herein by a) enzymatically digesting adipose tissue to obtain a population of adipose cells, wherein the population of adipose cells comprises at least one adipose stem cell (ASC); b) culturing adipose cells in a first culture medium at a seed density between 2 and 4×10⁵ cells/cm²; c) passaging the cells in the first culture medium at least once (e.g., 2, 3, 4, or 6 times); d) selecting cells having at least 90% (i.e., at least 90, 91, 92, 93, 94, 95, 96, 97, 98, 99, or 100%) expression of one or more pericyte markers; e) culturing the selected cells in a second culture medium, wherein the second culture medium is serum free and comprises at least one inflammatory cytokine; f) transferring the selected cells into a basal culture medium that does not contain inflammatory cytokines; g) removing cells from the basal culture medium to produce a cell-free conditioned medium comprising the secretome of the adipose cells; and h) lyophilizing the conditioned medium.

For example, the adipose tissue can be digested with collagenase.

Those skilled in the art will recognize that the one or more pericyte markers can be selected from CD140b, CD146, and/or neural/glial antigen 2 (NG2). Likewise, those skilled in the art will recognize that classical MSC markers may include CD73, CD90, and/or CD105.

In one embodiment, the second serum free culture medium contains between about 10 and about 30 ng/ml (i.e., 10, 11, 12, 13, 14, 15, 16, 17, 18, 19, 20, 21, 22, 23, 24, 25, 26, 27, 28, 29, or 30 ng/ml) TNFα, between about 1 and about 20 ng/ml IFNγ (i.e., 1, 2, 3, 4, 5, 6, 7, 8, 9, 10, 11, 12, 13, 14, 15, 16, 17, 18, 19, or 20 ng/ml), or a combination thereof.

Any of the culturing methodologies described herein (i.e., culturing in serum free media in the presence of TNFα and/or IFNγ) can synergistically increase the amounts of certain growth factors, cytokines, and/or other proteins present in the secretome of the adipose stem cells.

Metalloproteinase inhibitor 1 (“TIMP1”), a tissue inhibitor of metalloproteinases, is a glycoprotein known to be expressed in several tissues of organisms and has also been shown to promote cell proliferation in wide range of cell types. It may also exhibit anti-apoptotic functions.

Tumor necrosis factor-inducible gene protein (“TSG-6”) is a potent anti-inflammatory protein that has been implicated in ophthalmic animal disease models. For example, TSG-6 has been shown to inhibit the inflammatory response of microglial cells.

Culturing the cells in the second serum free culture medium containing at least one inflammatory cytokine increases TIMP1 expression by the cells. For example, TIMP1 expression may be increased by at least 2, 3, 4, 5, 6, 7, or more fold. (See Example 1, infra).

Culturing the cells in the second serum free culture medium containing at least one inflammatory cytokine also increases TSG-6 expression by the cells. For example, TSG-6 expression is increased by at least 2, 3, 4, 5, 6, 7, or more fold.

In these methods, culturing the cells in the presence of one or more inflammatory cytokines additionally decreases the T cell activity (multiplication) of the lyophilized composition. For example, T cell activity of the lyophilized composition is decreased by at least 2, 3, 4, 5, 6, 7, or more fold.

In some embodiments, the cells are removed from the second serum free culture medium after 24 hours.

The cells in the first culture medium can be passaged 2, 3, 4, or 5 times.

Effective tangential flow filtration of the cell-free conditioned media can be accomplished by adding an effective amount of EDTA to the conditioned media. In some embodiments, the conditioned media is concentrated prior to lyophilization. For example, this can be accomplished by filtering the conditioned media using tangential flow filtration (TFF) at a molecular weight cut off (MWC) of about 5 kDa.

The combination of the use of a specific tangential flow filtration protocol (see, e.g., Example 1, infra), the addition of effective amount of EDTA (e.g., 1 mM EDTA) to the ASC conditioned media prior to filtration, as well as the specific filter cut off (e.g., 5 kDa) allows the conditioned media to be processed in order to preserve and concentrate almost all of the ASC secreted proteins, nucleic acids, lipids, and/or polysaccharides, whether bound within or on the surface of extracellular vesicles such as exosomes or separate from the extracellular vesicles. In addition, this combination also removes small molecules and peptides found in the cell culture media. Together, this combination results in compositions that retain the therapeutic components of the soluble protein fraction and exosomal fraction. Likewise, the resulting secretome compositions are also superior to stem cell therapies, where a majority of injected cells are eliminated after injection. (See FIG. 13).

In further embodiments, following TFF filtering, the conditioned media is diafiltered into Tris EDTA buffer, histidine buffer, glycerine buffer, phosphate buffer, Tris HCl buffer, citrate buffer or a combination thereof prior to lyophilization. In another example, conditioned media may pass through centrifugal filters with defined molecular weight cut-off (MWC or WMCO) to concentrate. Those skilled in the art will recognize that any other suitable methods known in the art can be used to concentrate the conditioned media prior to lyophilization.

Also provided are methods of making the pharmaceutical compositions described herein by mixing an effective amount of the lyophilized composition with the sustained release drug delivery matrix to form a gel, paste-like, semi-solid, or microparticulate form of the drug composition. For example, the lyophilized composition may be reconstituted in the sustained release drug delivery matrix. Alternatively, the lyophilized composition may be reconstituted prior to mixing with the sustained release drug delivery matrix.

Those skilled in the art will recognize that the forming of the gel, paste-like, semi-solid, or microparticulate form of the pharmaceutical composition or any combination thereof can be accomplished by repeated cycles of pressing and folding, in an algorithmic manner, of the mixture of the sustained release drug delivery matrix and the lyophilized composition. The pressing may be accomplished by applying a pressure of not more than 10⁶ N.m⁻². Additionally, the hydrophobic matrix within the sustained release drug delivery matrix may be kept in a non-molten state throughout the mixing.

These methods may additionally involve the step of forming the pharmaceutical composition into a suitable dosage form (e.g., a dosage form suitable for intraocular injection).

Any of the aspects and embodiments described herein can be combined with any other aspect or embodiment as disclosed here in the Summary of the Invention, in the Drawings, and/or in the Detailed Description of the Invention, including the below specific, non-limiting, examples/embodiments of the present invention.

Unless otherwise defined, all technical and scientific terms used herein have the same meaning as commonly understood by one of ordinary skill in the art to which this application belongs. In the specification, the singular forms also include the plural unless the context clearly dictates otherwise.

Although methods and materials similar to or equivalent to those described herein can be used in the practice and testing of the application, suitable methods and materials are described below. All publications, patent applications, patents, and other references mentioned herein are incorporated by reference.

The references cited herein are not admitted to be prior art to the claimed application. In the case of conflict, the present specification, including definitions, will control. In addition, the materials, methods, and examples are illustrative only and not intended to be limiting.

Other features and advantages of the application will become apparent from the following detailed description in conjunction with the examples.

### BRIEF DESCRIPTION OF THE DRAWINGS

FIG. 1 shows the results of flow cytometric analysis of ASCs from one human donor.

FIG. 2A shows the SDS-PAGE/Filter results demonstrating effective secretome recovery and purification following tangential flow filtration and diafilitration of the process development run detailed in Example 1.

FIG. 2B shows that CC-101 contains both exosome and non-exosome associated proteins. Panel A shows quantification of antibody array spot intensities showing similar abundance of the cytokines present in the pre and post- 100 kDa molecular weight cut-off filtered CC-101. Panel B shows SDS-PAGE and immunoblot analyses of the retentate. 14-3-3, a protein incorporated into exosomes, and CD63, a tetraspanin incorporated into the lipid membrane of exosomes are enriched in the retentate.

FIG. 3A shows the results of the physical inspection of the lyophilizate in both histidine (HIS) buffer and Tris/EDTA (TE) buffer.

FIG. 3B shows an example of the proteins that are present and preserved pre- and post-lyophilization.

FIGS. 4A and 4B show product stability data for the pre- and post-lyophilized TE and HIS samples at 4° C. for 7 days.

FIG. 4C shows product stability for the post-lyophilized TE samples. Lyophilized CC101 was stored at room temperature, 4° C., or −80° C. for 21 days. Following incubation, samples were dissolved in 1 mL H₂O. Total protein and microRNA concentration were measured in triplicate using Qubit Protein Assay and Qubit microRNA kit, respectively.

FIG. 5 shows the results of DNA removal/Sartobind Q analysis.

FIG. 6A shows that CC-101 has immunosuppressive effects on stimulated CD4+ T-cells within stimulated peripheral blood mononuclear cells (PBMCs).

FIG. 6B shows that CC-101 has immunosuppressive effects on CD3/CD28 stimulated peripheral blood mononuclear cells.

FIG. 7A shows IFNγ/TNFα priming of ASCs increases the abundance of cytokines and chemokines in ASC-CM. 7A (Panel A) shows representative membrane-based antibody arrays comparing expression levels of many cytokines/chemokines from ASC-CM from cells untreated or treated with IFNγ and TNFα. Culture medium was used as a control for nonspecific background signal. 7A (Panel B) shows quantification of selected cytokine expression from 3 samples of ASC-CM from untreated or IFNγ/TNFα treated cells analyzed by antibody arrays. Note, to better illustrate the fold change in response to IFNγ/TNFα stimulation, the data has been background subtracted and normalized to the baseline cytokine expression from untreated cells.

FIG. 7B shows that culturing the cells in a second serum free culture medium containing IFNγ and TNFα has additive and synergistic effects on the expression of some proteins in ASC-CM. ASCs were stimulated with TNFα, IFNγ, TNFα and IFNγ, or untreated. 24 h after washout of the cytokines, the ASC-CM was collected and the protein composition was analyzed by label free shotgun proteomics.

FIGS. 7C and 7D show that shotgun proteomics and bioinformatic analyses reveal an abundance of exosome proteins in ASC-CM (CC-101).

FIG. 7E shows 100 abundant proteins identified in ASC-CM (pre-lyophilized) formulated in both histidine and Tris buffers and their post-lyophilized forms (CC-101) identified by LC-MS shotgun proteomics. NSAF×10⁵ values are represented as a heatmap.

FIG. 7F shows ELISA results demonstrating that paracrine factors released by ASC are unaffected by the lyophilization procedure.

FIG. 8 shows that culturing the cells in a second serum free culture medium with exogenously added TNFα, or IFNγ and TNFα increases the amount of TSG-6 expression in the final product.

FIG. 9A shows the concentration and size distribution of extracellular vesicles in CC-101 with Tris-EDTA buffer.

FIG. 9B shows the concentration and size distribution of extracellular vesicles in CC-101 with Histidine buffer.

FIG. 10 shows the release of the lyophilized composition from the sustained release drug delivery matrix.

FIG. 11 shows that the lyophilized composition resuspended in PBS improves visual acuity in mice following traumatic brain injury with 50 psi air blast to the brain.

FIG. 12 shows that the lyophilized composition resuspended in PBS improves visual contrast sensitivity in mice following traumatic brain injury with 50 psi air blast to the brain.

FIG. 13A is a series of photographs demonstrating that the lyophilized composition resuspended in PBS (CC-101) protects from hyper proliferation of the retinal pigment epithelium and vascular leakage when injected intravitreally in mice following traumatic brain injury with 50 psi air blast to the brain. Similar results were obtained with 8 animals in the CC-101 group.

FIG. 13B shows that the lyophilized composition resuspended in PBS (CC-101) reduces retinal GFAP levels in regions intermediate to the ONH and the ora serrata. Quantification of GFAP staining from photomicrographs shown on left shows significant reduction in GFAP fluorescence. Similar results were obtained with 4 animals in the CC-101 group.

FIG. 14 shows that the lyophilized composition is non-immunogenic and well tolerated in non-human primates following intravitreal dosing at Day 0 (64 μg/ml total protein) and Day 29 (128 μg/ml total protein).

FIG. 15 shows that CC-101 protects from vascular permeability in paracellular leakage assay.

FIG. 16 shows the proteins common to pre-lyophilized ASC-CM and reconstituted post-lyophilized CC-101, as determined by shotgun proteomics.

FIG. 17 is a list of the top miRNAs identified with RNA next gen sequencing of precipitated exosomes from pre-filtered ASC-CM.

### DETAILED DESCRIPTION OF THE INVENTION

### Definitions

The terms “treatment,” “treat,” or “treating,” and the like, as used herein covers any treatment of a human or nonhuman mammal (e.g., rodent, cat, dog, horse, cattle, sheep, and primates etc.), and includes preventing the disease or condition from occurring in a subject who may be predisposed to the disease or condition but has not yet been diagnosed as having it. It also includes inhibiting (arresting development of), relieving or ameliorating (causing regression of), or curing (permanently stopping development or progression) the disease or condition.

As used herein, the terms “a” or “an” means one or more or at least one.

As used herein, the term “about” refers to the recited value ±10%, ±9%, ±8%, ±7%, ±6%, ±5%, ±4%, ±3%, ±2%, or ±1%.

As used herein, a “therapeutically effective” or “effective” dosage or amount of a composition is an amount sufficient to have a positive effect on a given medical condition. If not immediate, the therapeutically effective or effective dosage or amount may, over period of time, provide a noticeable or measurable effect on a patient's health and well-being.

As used herein the phrase “adipose tissue” refers to a connective tissue which comprises fat cells (adipocytes).

As used herein a “pharmaceutical composition” refers to an effective amount of the lyophilized compositions described herein in combination with a sustained release drug delivery matrix. The pharmaceutical composition may optionally contain other components such as pharmaceutically suitable carriers and excipients, which may facilitate administration of a compound to a subject.

The term “pharmaceutically acceptable carrier” refers to a carrier or a diluent that does not cause significant irritation to a subject and does not abrogate the biological activity and properties of the administered compounds.

The term “excipient” refers to an inert substance added to a pharmaceutical composition to further facilitate administration of a compound.

As used herein, the terms “mix”, “mixing”, and the like describe a mechanical process or a mechanical treatment of the components. For example, mixing can be in the sense of carrying out repeated cycles of pressing and folding or comparable processing steps which lead to an intense compression and mixing of the provided hydrophobic matrices.

### Stem Cells

Adult stem cells can be harvested from a variety of adult tissues, including bone marrow, fat, and dental pulp tissue. While all adult stem cells are cable of self-renewal and are considered multipotent, their therapeutic functions vary depending on their origin. As a result, each type of adult stem cell has unique characteristics that make them suitable for certain diseases. Mesenchymal stem cells (MSCs) are multipotent, nonhematopoietic (non-blood) stem cells isolated from (derived from) a variety of adult tissues, including bone marrow and adipose tissue. As used herein, “isolated” refers to cells removed from their original environment. MSCs may differentiate into cells of mesodermal lineage, for example, adipocytes, osteoblasts, and chondrocytes.

Stem cells produce factors, such as growth factors, that regulate or are important for regulating multiple biological processes. A growth factor is an agent, such as a naturally occurring substance capable of stimulating cellular growth and/or proliferation and/or cellular differentiation. Typically, growth factors are proteins or steroid hormones. While the terms “growth factor” and “factor” and the like are used interchangeably herein, the term “biological factor” is not limited to growth factors.

Adipose stem cells (referred to interchangeably herein as “ASC” and “ADSL”), which are harvested from adult fat tissue and are present in high frequency relative to bone marrow stem cells, are best suited for treatment of vascular disease and soft tissue repair; bone marrow stem cells (BM-MSC) are best suited for treatment of inflammation and muscle damage; and dental pulp stem cells (DPSC) are best suited for neuroprotection.

Those skilled in the art will recognize that ASCs are good candidates for treating inflammatory and/or degenerative ophthalmic diseases because they possess numerous clinical advantages and offer a wide potential for clinical use. ASCs are obtained from a non-controversial tissue source; are applicable to many different degenerative diseases; are anti-inflammatory and immunoprivileged; are capable of differentiating into bone, cartilage, fat, muscle, heart, vascular, and nerve tissue types; and activate innate regenerative pathways in patients. (See Strem et al., Keio J Med 54(3):132-41 (2005), Traktuev et al., Circ. Res. 102:77-85 (2008), and Rajashekhar, Front Endocrinol (Lausanne) 5:59 (2014)).

For use in the compositions and methods described herein, donors can be identified who have expanded ASCs express high levels of CD140b, NG2, and/or CD146 pericyte protein surface markers at P5 prior to switching to serum free (SF) media for secretome harvest. In one non-limiting embodiment, donor inclusion criteria includes the following: non-smoking, female, under 30 years of age, family history of longevity on maternal and paternal sides of family, and/or no known illnesses or significant family history of chronic disease.

ASC also display similar stem cell surface markers as pericytes (e.g., CD140b, CD146, NG2, and/or 3G5 ganglioside antigen), which may possibly be due to their association with vasculature within fat. ASC also play a key role in the regeneration and formation of new blood vessels. Because ASC are multipotential mesenchymal progenitor cells and have phenotypic overlap with pericytes that encircle micro-vessels in multiple human organs (including adipose tissue), these cells have a direct role in providing microvascular support.

Human mesenchymal stem cells (MSCs), including ASCs, are characterized by the surface marker profile of CD45−/CD31−/CD73+/CD90+/CD105+/CD44+ (or any suitable subset thereof). (See Bourin et al., Cytotherapy 15(6):641-648 (2013)). Further, appropriate stem cells display the CD34+ positive at the time of isolation, but lose this marker during culturing. Therefore the full marker profile for one stem cell type that may be used according to the present application includes CD45−/CD31−/CD73+/CD90+/CD105+. In another embodiment utilizing mouse stem cells, the stem cells are characterized by the Sca-1 marker, instead of CD34, to define what appears to be a homologue to the human cells described above, with the remaining markers remaining the same.

### ASC Culture Conditioned Medium (ASC-CM)

Provided herein are conditioned medium (CM) including biological factors secreted by ASCs (also referred to herein as the “secretome”, “ASC-CM”). Also provided are processed conditioned medium including biological factors secreted by ASCs that have been filtered through tangential flow filtration (also referred to herein as “Post-TFF ASC-CM” or “Pre-lyo ASC-CM”) as well as lyophilized processed conditioned medium comprising biological factors secreted by ASCs that have been filtered through tangential flow filtration (also referred to herein as the “CC-101”, “CC101”).

The conditioned medium is obtained by culturing stem cells in media, as described herein, and separating the resulting media, which contains stem cells and their secreted stem cell products (secretome) into conditioned medium that contains biological factors and fewer stem cells than were present prior to separation. The conditioned medium may be used in the methods described herein and is substantially free of stem cells (may contain a small percentage of stem cells) or free of stem cells. Biological factors that may be in the conditioned medium include, but are not limited to, proteins (e.g., cytokines, chemokines, growth factors, enzymes), nucleic acids (e.g., miRNA), lipids (e.g., phospholipids), polysaccharides, and/or combinations thereof. Any combination(s) of these biological factors may be either bound within or on the surface of extracellular vesicles (e.g., exosomes) or separate from extracellular vesicles.

Conditioned medium (and, thus, stem cell secreted factors) can be obtained from stem cells obtained from the individual to be treated (the individual in need) or from another (donor) individual, such as a young and/or healthy donor). For example, ASC obtained from the individual to be treated (autologous stem cells) or from a donor (allogeneic stem cells), can be used to produce the conditioned medium described herein.

Adipose tissue derived adherent cells may be isolated by a variety of methods known to those skilled in the art. For example, such methods are described in U.S. Pat. No. 6,153,432, which is incorporated by reference. The adipose tissue may be derived from omental/visceral, mammary, gonadal, or other adipose tissue sites. One preferred source of adipose tissue is omental adipose. In humans, the adipose is typically isolated by liposuction. For example, approximately 150-300 ml of abdominal adipose tissue can be extracted via liposuction.

As outlined in Example 1, infra, adipose tissue digestion is accomplished using minor modifications made to standard tissue digestion protocols known in the art. Preferably, these modifications are ones that help to increase overall P0 ASC yield.

Cell culture is performed using standard cell culture process. Determination of the appropriate cell culture process is within the routine level skill in the art.

At P5, cells are switched to serum free (SF) media. Multiple inflammatory factors are added in the SF media stage to stimulate the cells for 24 hrs and then removing and rinsing the cells before culturing the cells without any fetal bovine serum (FBS) or other inflammatory factors. The combined addition of IFNγ and TNFα increased TIMP1 expression 7 fold, which is believe to be correlated with greater therapeutic potency vis a vis our degenerative vascular target.

Other cytokines and/or cell signaling mediators may also be added to the SF culture media (either alone or in any combinations). For example, cytokines and/or cell signaling mediators that may be added can include, but are not limited to IFNγ, TNFα, interleukin-1b (IL-1b), interleukin-2 (IL-2), interleukin-6 (IL-6), interleukin-8 (IL-8), interleukin-10 (IL-10), interleukin-18 (IL-18), transforming growth factor-b (TGF-b), granulocyte colony-stimulating factor (G-CSF), granulocyte macrophage colony-stimulating factor (GM-CSF), platelet-derived growth factor (PDGF), nitric oxide (via NO-donor molecules) and/or hydrogen peroxide.

In one embodiment, there is a quick transfer of the ASC-CM into 10 mM EDTA. The addition of EDTA is important for maintaining the integrity and separation of the thousands of proteins and miRNA present in the ASC-CM during filtration.

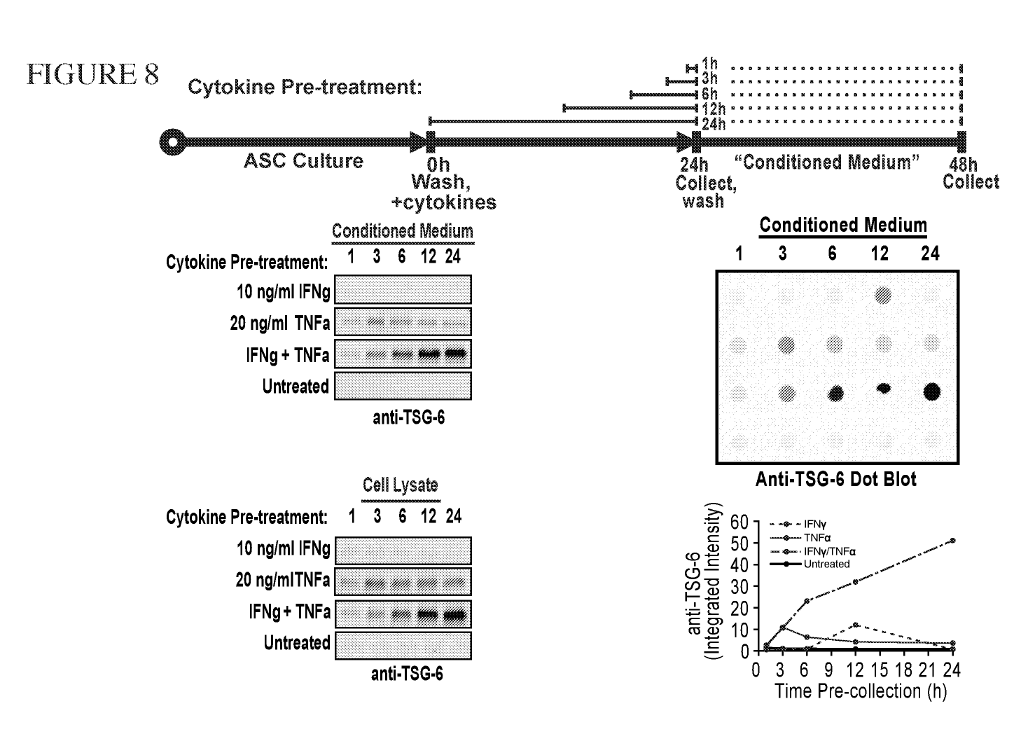

Culturing the cells in the second serum free culture medium containing at least one inflammatory cytokine also increases TSG-6 expression by the cells. For example, TSG-6 expression is increased by at least 2, 3, 4, 5, 6, 7, or more fold. (See FIG. 8).

Next, ASC-CM is concentrated and diafiltered by TFF. Major aspects of the processing that occurs at this stage include the use of a 5 kD filter cutoff for TFF and combination of TFF and diafiltration.

Additional purification steps may also be taken to further concentrate solution and Sartobind Q filtration to remove DNA.

Next, the ASC-CM is lyophilized to form the lyophilized compositions described herein. Lyophilization cycle must be very slow and conservative in order for cake to form. It is important to use a buffer that works for both diafiltration and secreted factor preservation as well as for lyophilization of dilute secretome solution.

Non-limiting examples of base media useful in culturing according to the present invention include Minimum Essential Medium Eagle, ADC-1, LPM (Bovine Serum Albumin-free), F10 (HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Alpha (MEM-alpha), Minimum Essential Medium Eagle (MEM-E-with Earle's salt base), Minimum Essential Medium Eagle (MEM-H-with Hank's salt base) and Minimum Essential Medium Eagle (MEM-NAA with non- essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. A preferred medium for use in the present invention is MEM-alpha. These and other useful media are available from GIBCO, Grand Island, N.Y., USA and Biological Industries, Bet HaEmek, Israel, among others. A number of these media are summarized in Methods in Enzymology, Volume LVIII, “Cell Culture”, pp. 62 72, edited by William B. Jakoby and Ira H. Pastan, published by Academic Press, Inc.

The medium may be supplemented with serum such as fetal serum of bovine or other species, and optionally or alternatively, growth factors, cytokines, and hormones at concentrations of between picograms/ml to milligram/ml levels. For example, the medium may be supplemented with inflammatory cytokines (e.g., IFNγ and/or TNFα) in order to stimulate the cells.

It is further recognized that additional components may be added to the culture medium. Such components may be antibiotics, antimycotics, albumin, amino acids, and other components known to the art for the culture of cells. Additionally, components may be added to enhance the differentiation process when needed.

### Biomimetic Regenerative Medicine Platform

Most cell types secrete various regenerative factors (e.g. cytokines, chemokines, growth factors, and the like) that are collectively known as the secretome, which function as messengers for cell-to-cell communication.

Stem cells' primary means of effecting regeneration is believed to occur through cell-to-cell signaling, rather than through transplantation. As a result, disclosed herein is a cell-free, anti-inflammatory, regenerative medicine platform that can mimic the function and regenerative signaling mechanisms of any stem cell and that has the practicality and economics of a conventional drug. This biomimetic technology is designed to release regenerative and anti-inflammatory factors derived from stem cells in order to stimulate tissue regeneration and vascular repair in the same manner as a live stem cell.

Secreted regenerative factors are extracted from culture expanded stem cells and refined. Specifically, tissue (e.g., fat tissue) is processed and the supernatant is cultured with growth media until the cell population reaches confluence and is primarily stem cells.

In some embodiments, the factors secreted by the stem cells can be administered directly to patients. However, the factors from the secretome are combined with an effective amount of a lyophilizing agent prior to lyophilization. The resulting lyophilized compositions can be reconstituted prior to administration to the patient.

In other embodiments, the lyophilized composition is combined with generally regarded as safe (GRAS) excipients (Therakine, Berlin), such as those disclosed in U.S. 20140356435, which is herein incorporated by reference in its entirety, that function as a sustained release drug delivery matrix, in order to form the pharmaceutical compositions described herein. Specifically, suitable excipients (e.g., hydrophobic excipients) are chosen and combined with active pharmaceutical ingredients and are homogenized through folding, kneading, pressing, and/or cutting. Those skilled in the art will recognize that the precise excipients and the formulation techniques utilized can be adjusted in order to fine-tune product specifications, including, for example, secretome release, duration, shape, and/or size. Determination of the appropriate excipient(s) is within the routine level of skill in the art.

The resulting product is a biomimetic regenerative therapy that releases stem cell derived factors following injection into a target tissue niche.

Using this biomimetic regenerative therapy, a persistent, linear release of stem cell factors can be achieved for up to 6 months (e.g., up to 1, 2, 3, 4, 5, or 6 months) or more. Moreover, the efficient delivery of just the regenerative stem cell factors reduces manufacturing and dose costs that are associated with other cell-based regenerative therapies. Likewise, using this biomimetic regenerative therapy, discrete and quantifiable in vivo dosing is possible; production is readily scalable; and the off-the-shelf product, which only requires standard refrigeration, can easily be manufactured, stored, and administered.

This biomimetic platform utilizes a biodegradable sustained release drug delivery matrix that utilizes the breakdown of physical rather than chemical bonds to achieve this sustained release of a complex assortment of regenerative proteins for periods up to six months without negatively impacting the potency of the proteins and/or extracellular vesicles. Specifically, these matrices are based on nanoscale physical chemistry and biophysical interactions and are mechanically formed through macroscopic processing. Because there is no chemical cross-linking, chemical or biological changes to the active therapeutic ingredients as well as extreme pH and/or heat conditions, the use of these drug delivery matrices does not denature the proteins that are contained within them. Accordingly, the use of these sustained release drug delivery matrices allows for selective, site-specific delivery; reduction of drug toxicity; improvements in safety and efficacy; linear release of drugs with durations ranging from weeks to months; direct administration into affected organs; bolus release followed by a linear release up to 6 months or more; the use of lower concentration of active therapeutic ingredients or cells; and/or the preservation of bioactivity throughout manufacturing and delivery. Moreover, they do not result in the acid burst that is observed with the use of PLGA delivery systems.

Thus, this sustained release drug delivery matrix can accommodate a complex mixture of regenerative factors, such as those found in the secretome of adipose stem cells. Therefore, the biomimetic regenerative medicine platform described herein can be administered approximately every 3-6 months, as compared to standard biologic treatments, which must be delivered approximately every 2-8 weeks, thereby reducing patient inconvenience, health care costs, and/or risk exposure.

### Sustained Release Drug Delivery Matrix

Provided herein are pharmaceutical compositions containing an effective amount of a lyophilized composition and a sustained release drug delivery matrix. Such pharmaceutical compositions can be manufactured by providing at least the lyophilized composition and a hydrophobic matrix; and mixing the hydrophobic matrix and the lyophilized composition to form a gel, a paste-like, semi-solid or microparticulate form of pharmaceutical composition or a combination thereof. An advantage of this manufacturing method is that it provides a sustained release formulation with improved release characteristics. Importantly, the resulting pharmaceutical compositions allow the sustained release of ingredients characterized by a specific biological activity that might decrease or terminate when using other delivery mechanisms.

By way of non-limiting example, the hydrophobic matrix itself can be comprised of natural waxes, fats and oils, tocopherols and derivatives thereof, as well as synthetic substances or chemically modified natural waxes, fats, and/or oils.

In some embodiments, the hydrophobic matrix is formed by mixing at least a hydrophobic solid component and a hydrophobic liquid component, which allows the formation of hydrophobic matrices having a wide range of consistencies i.e., rheological properties like viscosities of the paste-like or semi-solid composition depending on their quantitative relation. Selection of suitable liquid and solid hydrophobic components allows for the formation of gels, paste-like compositions, or semi-solid compositions having the desired properties.

In various embodiments, the ratio between the solid hydrophobic phase and the liquid hydrophobic phase of the above embodiments is greater than or equal to 0 and less than or equal to 100, particularly greater than or equal to 0.5 and less than or equal to 50, more particular greater than or equal to 1 and less than or equal to 20, and even more particular greater than or equal to 1 and less than or equal to 10.

The pharmaceutical compositions may optionally also contain at least one excipient, which can act as a buffer, filler, binder, osmotic agent, lubricant, and/or fulfill similar functions. For example, the excipient may be selected from monosaccharides, disaccharides, oligosaccharides, polysaccharides like hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and/or mixtures or combinations thereof The presence of such excipients may further modify the release characteristics of the sustained release drug delivery matrix.

The hydrophobic materials can optionally be labeled with any of a wide variety of agents, which are known to those skilled in the art. For example, dyes, fluorophores, chemiluminescent agents, isotopes, metal atoms or clusters, radionuclides, enzymes, antibodies, or tight-binding partners such as biotin and avidin can all be used to label the hydrophobic materials for detection, localization, imaging, or any other analytical or medical purpose. The hydrophobic materials, particularly a liquid component of the matrix, can also optionally be conjugated with a wide variety of molecules in order to modify its function, modify its stability, and/or further modify the rate of release. The pharmaceutical composition can be coated with a covalently- or non-covalently-attached layer of a species such as small molecules, hormones, peptides, proteins, phospholipids, polysaccharides, mucins, or biocompatible polymers such polyethylene glycol (PEG), dextran, or any of a number of comparable materials. The wide range of materials, which can be used in this fashion, and the methods for accomplishing these processes, are well known to those skilled in the art.

The pharmaceutical compositions can be formed by repeated cycles of pressing and folding, e.g., pressing and folding in an algorithmic manner of the hydrophobic matrix itself and/or mixed with the lyophilized composition. The folded mass is then pressed again. By repeating these processes, a better distribution of the pharmaceutically active compound (API) (i.e., the secretome of the ASCs) throughout the matrix can be achieved. For example, kneading is one example of an algorithmic pressing-folding cycle. The pressing may be accomplished by applying a pressure of not more than 10⁶ N.m⁻².

The controlled mixing of the components into a homogeneous mass transforms the preparation into a paste- or dough-like consistency, which is appropriate for the production of slow release compositions. For example, all solid hydrophobic ingredients can be mixed in a first step followed by adding the liquid hydrophobic matrix component to generate the paste-like or semi-solid consistency during mechanical treatment. The lyophilized composition is added, for instance as a dry powder or a liquid or aqueous solution into the paste like mass and the mechanical treatment is continued to gain homogeneity of the paste like mass.

The matrix formed by the mechanical treatment of solid and liquid components is typically a hydrophobic matrix but may also include a small amount of hydrophilic excipients/ingredients and/or aqueous solutions.

In some embodiments, no heating to transfer the hydrophobic solid component into a liquid state is used, and the solid hydrophobic matrix is kept throughout the mechanical treatment in a non-molten state.

In other embodiments, in order to prevent self-organization processes from occurring, active cooling is used in order to keep the hydrophobic matrix in a non-molten state throughout the pressing and folding cycles.

For example, the temperature of the mixture during pressing and folding cycles can be kept below a certain temperature value (e.g., below 37, below 45, below 50, or below 60° C.) by cooling, which protects susceptible biologically active molecules (e.g., proteins in the secretome) from denaturation.

Prior to mixing with the hydrophobic matrix, the lyophilized composition can be reconstituted using any suitable reconstitution methods known in the art. Alternatively, the lyophilized composition does not need to be reconstituted prior to mixing with the hydrophobic components.

Examples of suitable solid hydrophobic components include, but are not limited to, waxes, fruit wax, carnauba wax, bees wax, waxy alcohols, plant waxes, soybean waxes, synthetic waxes, triglycerides, lipids, long-chain fatty acids and their salts like magnesium stearate, magnesium palmitate, esters of long-chain fatty acids, long-chain alcohols like cetyl palmitate, waxy alcohols, long-chain alcohols like cetylalcohol, oxethylated plant oils, oxethylated fatty alcohols, and combinations thereof

Examples of suitable liquid hydrophobic components include, but are not limited to, plant oils, castor oil, jojoba oil, soybean oil, silicon oils, paraffin oils, and mineral oils, cremophor, oxethylated plant oils, oxethylated fatty alcohols, tocopherols, lipids, phospholipids.

After formation, any of the pharmaceutical compositions described herein can be further processed into a suitable form, such as, for example, bodies or micro-particles of desired shape, size and size distribution by means of colloid forming techniques and other technological procedures. Colloid forming techniques comprise e.g. milling, cold extruding, emulgating, dispersing, sonicating.

The pharmaceutical compositions formed by the methods described herein maintain their drug-releasing properties for a prolonged time such as weeks and months. Thus, the lyophilized composition (whether reconstituted prior to mixing or not) remains protected in the paste-like or semi-solid mixture so that its specific biological activity can be maintained.

If desired, additional barrier layers can be formed around the pharmaceutical compositions. For example, a micro-porous membrane made from ethylene/vinyl acetate copolymer or other materials for ocular use can be formed around the paste-like or semi-solid mixture. Further options include, for example, the use of biodegradable polymers for subcutaneous and intramuscular injection, bioerodible polysaccharides, hydrogels etc.

In some embodiments, the effective amount of the lyophilized composition within the pharmaceutical composition may be between about 0.01 and about 25% (w/w) (e.g., 0.01, 0.02, 0.03, 0.04. 0.05, 0.06. 0.07, 0.08, 0.09, 0.1, 0.2., 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25% (w/w). In other embodiments, the amount of the lyophilized composition in the pharmaceutical composition will be much higher (i.e., 25, 30, 35, 40, 45, 50, 55, 60% (w/w) or more).

Those skilled in the art will recognize that the percentage of active (i.e., the ASC secretome) within the pharmaceutical composition will be about 0.1 to about 10%.

The various starting components such as the hydrophobic matrix and/or the lyophilized composition can be further manipulated and processed using a wide variety of methods, processes, and equipment familiar to one skilled in the art. For example, the hydrophobic matrix components can be thoroughly mixed using any of a number of known methods and equipment, such as trituration with a mortar and pestle or blending in a Patterson-Kelley twin-shell blender, before adding the API. Furthermore, a wide variety of shapes, sizes, morphologies, and surface compositions of the pharmaceutical composition can be formed. Micro-particles or cylindrical bodies with different aspect ratios can be prepared by means of mechanical milling, molding, and extruding or similar processes of the paste-like or semi-solid or even semi-solid material. The resulting particles can be further treated to prepare them for specific applications such as, for example, drug delivery systems. The mixture, paste or mass can also be transformed into micro-particles or bodies by means of cold extrusion, cooled pressure homogenization, molding, and/or other such well-established procedures can yield a wide range of final products. As another example, the pharmaceutical composition can be squeezed through a sieving disk (i.e., a die) containing predefined pores or channels with uniform pore geometry and diameter by an extrusion process.

### Therapeutic Uses

Both the lyophilized compositions and the pharmaceutical compositions described herein are applicable to a wide range of degenerative and inflammatory diseases for which effective treatment solutions are lacking. For example, they are well-suited for ocular disease targets such as retinal diseases, including, but not limited to diabetic retinopathy, age-related macular degeneration, glaucoma, retinitis pigmentosa, retinopathy of prematurity, iritis, uveitis, Stargardt's disease and traumatic brain injuries, traumatic injuries of the brain effecting vision and/or the retina because they are able to address retinal disease at its source.

In normal retinal vessels, pericytes help to stabilize vessels in the retina by lining endothelial cells. However, in retinopathy, inflammation and oxidative stress due to age or high blood sugar can lead to the death of pericytes and the degeneration of the vessel lining, which causes vascular leakage of growth factors and the proliferation of unwanted blood vessels in the retina. (See Cheung et al., Lancet 376(9735):124-36 (2010); Rehman, J. Mol. Med (Berl) 89:943-45 (2011); Wei, et al., Stem Cells 27:478-88 (2009); and Antonetti, Nat Med 15:1248-49 (2009)).

As demonstrated in Rajashekhar et al., PLoS One 9(1):e84671 (2014) (herein incorporated by reference), delivery of regenerative factors derived from adipose stem cells results in retinal regeneration and vascular repair. In two animal models, intravenous injection of adipose stem cells (ASCs) and/or factors secreted therefrom (without cells) improved retinal function, conferred neuroprotection by release of trophic factors; alleviated vascular leakage by direct differentiation into pericytes, and reduced inflammation by down regulating key inflammatory genes including but not limited to ICAM-1. (See Gene ID: 3383). The injection of derived secreted factors produced comparable results when compared to the injection of live stem cells.

Intravitreal injection of mesenchymal stem cells (MSC) has also been shown to confer neuroprotection of retinal pigment epithelial cells (RPE) and retinal ganglion cells in a laser induced open angle glaucoma animal model. (See Ezquer et al., Stem Cell Res Ther 7:42 (2016). In fact, it is well established that MSCs can secrete a range of neurotrophic factors including, but not limited to, nerve growth factor (NGF), brain derived neurotrophic factor (BDNF), glial derived neurotrophic factor (GDNF), and ciliary neurotrophic factor (CNF). (See Johnson et al., IOVS 51:2051-59 (2010)). Further, the injection of bone marrow derived MSCs into the anterior chamber of the eye in a laser induced open angle glaucoma animal model induces trabecular meshwork and reduces intraocular pressure. (See Yu et al., Biophysical and Biochemical Research Communication 344(4):1071-79 (2006) and Kelley et al., Exp Eye Res. 88(4):747-51 (2009)).

Key regenerative attributes of ASCs include, for example, repair of leaking retinal blood vessels by replacing lost pericytes; secretion of a number of neurotrophic and anti-apoptotic factors; protection and repair of retinal epithelial cells and retinal ganglion cells; reduction of inflammation, thereby promoting growth; and/or induction of trabecular meshwork regeneration and reduction of intraocular pressure.

Any of the compositions described herein can easily be injected through common techniques into the vitreous chamber of the eye. For example, the delivered regenerative factors are released from the biomimetic pharmaceutical compositions in order to protect and/or stimulate the regrowth of pericytes and astrocytes, which promotes regeneration.

The significant clinical and manufacturing efficiencies of the compositions described herein include, but are not limited to the following:
a true off-the-shelf product that is easy to store and handle: following lyophilization, the active cell derived therapeutic can be stored at room temperature and has a shelf-life comparable to existing biologic drugs on the market. Moreover, no cryopreservation is required, and the final pharmaceutical product can be refrigerated and stored for several weeks to months;
controlled dosing, sustained release: combination of the lyophilized composition with the sustained release drug delivery matrix to produce the pharmaceutical compositions provides superior control over where and when regenerative factors are released and allows quantification and standardization of dosing to an unprecedented degree in the regenerative medicine space;
reduced immunogenicity: because stem cells must come from a donor, significant questions remain regarding their long-term compatibility with recipient tissue. (See Eliopolous et al., Blood 106:4057-4065 (2005); Hare et al., JAMA 308:2369-79 (2012); Huang et al., Circulation 122:2419-29 (2010); and Richardson et al., Stem Cell Rev 9:281-302 (2012)). However, the cell-free compositions described herein release similar factors as a stem cell regardless of the immune system response;
non-invasive targeting of ocular tissues: because the blood retinal barrier creates a challenge for conventional drug delivery, invasive procedures are still common practice. Thus, there is a continued need to develop ocular drug delivery systems that can deliver drugs to its target without requiring undue risk exposure. (See Guadana et al., The AAPS Journal 12(3) (2010));
increases bioavailability: more than 90% of the marketed ophthalmic formulations are in the form of eye drops, which are unable to reach the retina. (See Roots Analysis, Sustained Release Ocular Drug Delivery Systems 2014-2024 (2013)). As such, increasing the therapeutic dose of a treatment that reaches the back of the eye and having a safe and convenient way of doing are important factors in treating and managing retinal disease.
releases therapy at a slow, constant, controlled rate: conventional discrete delivery of small molecules or biologics through drops or injections means that drug levels are oscillating and, therefore, management of condition is inconsistent. In contrast, the pharmaceutical compositions described herein prolong release time and allow for stable, linear release of therapeutic proteins and factors for periods up to six months;
improves patient compliance: most ocular treatments (like eye drops and suspensions) call for daily topical administration. Therefore, it is common for patients to miss treatments, which ultimately can lead to disease management issues. In contrast, patients will likely only require one injection of the pharmaceutical compositions described herein from their doctor about every three months. Therefore, they will receive the continuous benefit without the risk or hassle of daily drops or bimonthly injections.

The lyophilized or pharmaceutical composition may be administered in a systemic manner. Alternatively, one may administer the pharmaceutical composition locally, for example, topically or via injection directly into a tissue region of a patient.

For injection, the active ingredients of the pharmaceutical composition may be micronized and/or formulated in aqueous solutions, preferably in physiologically compatible buffers such as Hank's solution, Ringer's solution, or physiological salt buffer. For topical administration, penetrants appropriate to the barrier to be permeated are used in the formulation. Such penetrants are generally known in the art.

Any of the compositions described herein can be administered into the human or animal body, for example, by implanting or injecting the mixture into a human or animal body; intraocular injecting the mixture into a human or animal body; subcutaneous injecting the mixture into a human or animal body; intramuscular injecting the mixture into a human or animal body; intraperitoneal injecting the mixture into a human or animal body; intravenous injecting the mixture into a human or animal body; oral administration of the mixture into the human or animal body; intramuscular injecting the mixture into the human or animal body; intrathecal injecting the mixture into the human or animal body; sublingual administration of the mixture into the human or animal body; buccal administration of the mixture into the human or animal body; rectal administration of the mixture into the human or animal body; vaginal administration of the mixture into the human or animal body; ocular administration of the mixture into the human or animal body; otic administration the mixture into the human or animal body; cutaneous administration the mixture into the human or animal body; nasal administration (i.e., by spraying) the mixture into the human or animal body; cutaneous administration the mixture into the human or animal body; topical administration the mixture into the human or animal body; systemic administration the mixture into the human or animal body; transdermal administration the mixture into the human or animal body; and/or inhalative or intranasal (i.e., by nebulization) administration of the mixture into the human or animal body.

For any of the compositions described herein, the effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1).

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. Determination of the appropriate amount is within the routine level of skill in the art.

Any of the compositions described herein may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

### Vascular Repair

Diabetic retinopathy develops as sustained metabolic dysregulation, which inflicts progressive damage to the retinal microvasculature. This, in turn, increases vascular permeability. In advanced stages, it can lead to the aberrant proliferation of vascular endothelial cells, which damage the rods and cones of the retina, thereby causing vision loss.

Previous studies have shown that increased vascular permeability in diabetic rats decreased with intravitreal adipose stem cell or secretome injection.

### Neuro-Protection

Gliosis is a nonspecific reactive change of Muller glial cells in response to damage to the retina. During retinal development Muller glia arise from neural retinal progenitor cells and span nearly the entire width of the retina from the outer limiting membrane, where Müller processes form connections with photoreceptors, to the inner limiting membrane, where Müller and retinal astrocyte processes form the boundary between the retina and the vitreous. (See Newman et al., Trends Neurosci. 19:307-312 (1996)). Both the Müller glia and retinal astrocytes have been shown to play very important roles in supporting and protecting the retinal neurons, specifically are critical to the formation of the blood-retinal barrier. (See Kuchler-Bopp et al., Neuroreport. 10:1347-1353 (1999)). Reactive gliosis involves the proliferation or hypertrophy of several different types of glial cells, including astrocytes, microglia, and oligodendrocytes in diseases including glaucoma, retinal ischemia, and diabetes. (See Bringmann et al., Prog Retin Eye Res. 25:397-424 (2006)). In its most extreme form, the proliferation associated with gliosis leads to the formation of a glial scar. (See Silver Jet al., Nat Rev Neurosci. 5:146-156 (2004)).

Gliosis is notably decreased following injury to the retina when adipose derived mesenchymal stem cells or their regenerative factors are administered, as evidenced by a reduction in GFAP and Casp-3 expressing cells, which are gene expression markers associated with gliosis. Previous studies have shown that increased gliosis in ischemic reperfusion rat model decreased with intravitreal injection of adipose stem cells and adipose stem cell secretome. Further evidence that adipose stem cell secretome reduce gliosis comes from suppression of microglial activation. Activated microglia exist in two states; an M1-state associated with production of pro-inflammatory cytokines and reactive oxygen species or an anti-inflammatory M2-state associated with wound healing and debris clearance. Activated microglia treated with ASC secretome demonstrated a decrease in microglial activation.

### Improved Retinal Function

The electroretinogram (ERG) is evoked from the retina of the eye by a brief flash of light and measures the electrical response of retinal cells including photoreceptors (rods and cones), inner retinal cells (bipolar and amacrine cells), and the ganglion cells. Thus, the ERG is a test that helps evaluate retinal function and diagnose a number of retinal disorders, including diabetic retinopathy.

Previous studies have shown poor response of electroretinogram in an ischemic reperfusion rat model has been shown to be alleviated with intravitreal injection of adipose stem cells and adipose stem cell secretome.

### Anti-Inflammatory

ASC have been shown to significantly reduce inflammation at the site of injury by secreting factors that prevent the proliferation and function of many inflammatory immune cells, including T-cells, natural killer cells, B cells, monocytes, macrophages, and dendritic cells.

Several pro-inflammatory cytokines and biomarker panel genes that are implicated in diabetic retinopathy research (e.g., ccl2, ICAM-1, Edn2, TIMP1, Crybb2, Gat3, Lama5, and Gbp2) were significantly downregulated in the diabetic rat model with a single intravitreal injection of ASCs. Previous studies have demonstrated that increased diabetic retinopathy related gene transcripts in diabetic rats decreased with intravitreal adipose stem cell injection.

### Kits, Medicaments and Articles of Manufacture

Any of the compositions described herein may be used in the manufacture of the medicament, for example, a medicament for treating or prolonging the survival of a patient suffering from a disease, condition, or disorder.

Also provided are kits for treating or prolonging the survival of a patient with a disease, condition, or disorder containing any of the compositions described herein, optionally along with instructions for use.

Articles of manufacture and dosage forms are also provided, which include a vessel containing any of the compositions described herein and instructions for use to treat or prolong the survival of a patient with a disease, condition, or disorder.

Any of the compositions described herein can be included in a container, pack, or dispenser together with instructions for administration.

The invention having been described, the following examples are offered by way of illustration and not limitation.

### EXAMPLES

### Example 1

### Development of an Adipose Tissue Derived Mesenchymal Stromal Cell Conditioned Media Product Under cGMP Guidelines

### Manufacturing Protocol

### Donor Selection and Tissue Harvest

Approximately 150-300 ml of abdominal tissue extracted via liposuction can be selected from a suitable donor (e.g., non-smoking, female, under 30, family history of longevity on both sides of family, and/or no known illnesses or significant family history of chronic disease.

### Digestion

Adipose tissue digestion is accomplished using minor modifications made to standard tissue digestion protocols known in the art. Preferably, these modifications are ones that help to increase overall P0 ASC yield.

Approximately 300 ml lipoaspirate is transferred to a sterile bottle and allow the adipose tissue to settle above the blood fraction. Blood is removed from beneath the adipose tissue using a 10 ml aspirator pipet, and lipoaspirate is rinsed with 300 ml of DPBS by shaking the bottle vigorously for 10 seconds.

Adipose tissue is allowed to float above DPBS and then remove DPBS with a 10 ml aspirator. These steps are repeated three additional times. More rinses may be required if the final rinse DPBS is not clear.

Next, 2X Liberase MNP-S (0.14 WU/ml) is prepared. Lipoaspirate is divided into 50 ml tubes, and equal volume of 2X Liberase is added and shaken vigorously for 5-10 seconds to ensure proper mixing.

Lipoaspirate is then incubated at 37° C. on a nutating mixer with orbital rotations at 24 rpm/min for 90 minutes. To stop enzyme activity FBS is added to a final concentration of 10%, and mixed well.

Next, the solution is centrifuged at 300 g for 10 minutes, and the floating adipocytes, lipids and digestion medium is aspirated.

The pellet is the Stromal Vascular Fraction (SVF) of the adipose tissue, which is resuspended in ACK Lysis Buffer for RBC lysis and incubated at RT for 5-10 minutes.

Following centrifugation at 300 g for 10 minutes, the supernatant is aspirated. Then, the pellet is resuspended in 10 ml Complete Medium (α-MEM+10% FBS+Glutamax), and the cell resuspension is passed through a 100 um cell strainer to remove undigested tissue clumps. The strainer is rinsed with 5 ml Complete Medium.

The cell suspension is next passed through a 40 um cell strainer, and the filter is rinsed with 5 ml Complete Medium.

Finally, the cell suspension is centrifuged at 300 g for 10 minutes, and the cell pellet is resuspended in Complete Medium and the cells are counted.

### Cell Culture: P0-P2

Cells are seeded at a density of 2-4×10⁵ cells/cm² in appropriate size T flasks to form the P0 culture. Cell cultures are checked the next day, and a half feed is performed on the 1^{st} or second day. Cultures are fed every 3-4 days.

When colonies start getting dense, harvest cultures with TryPLE, count cells and seed P1 at 5×10³ cells/cm² in appropriate size T flasks. Cells are again fed every 3-4 days and harvested when cells are 80-90% confluent.

Cells are cryopreserved to make a P2 RCB at 1-2×10⁶ cells/ml/vial.

### Cell Culture: P2 Dethaw—P5

Cell culture is performed using standard cell culture process, and determination of the appropriate cell culture process is within the routine level skill in the art.

P2 ASCs are thawed and cultured in 4× T225 flasks. When 80-90% confluent, P2 cells were harvested and sub-cultured into 6× single trays at P3; when 80-90% confluent, P3 cells were harvested and sub-cultured into 2×10 tray cell factories at P4; when 80-90% confluent, P3 cells were harvested and sub-cultured into 2×10 tray cell factories at P4; and when 80-90% confluent, P4 cells were harvested and sub-cultured into 10× 10 tray cell factories (10 CFs) at P5.

### P5 Switch to Serum Free Media

At P5, cells are switched to serum free (SF) media. Multiple inflammatory factors are added in the SF media stage to stimulate the cells for 24 hrs. The cells are then removed and rinsed before culturing without any FBS or other inflammatory factors such as IFNγ and/or TNFα. In one embodiment, there is a quick transfer of the ASC-CM into 10 mM EDTA.

At 80% ASC confluence, 10 CFs were rinsed twice with DPBS—(without Ca2+ Mg2+). 20 ng/ml TNFα and 10 ng/ml IFNγ supplemented Serum Free Medium (SFM) was added to 10 CFs. Twenty four hours later, 20 ng/ml TNFα and 10 ng/ml IFNγ supplemented SFM was discarded and cells were rinsed twice with DPBS.

Next, SFM (unsupplemented) was added to each 10 CFS, and twenty four hours later, ASC-CM was harvested and collected in 500 ml centrifuge bottles, which were centrifuged at 2000 g for 10 minutes (to remove large debris).

ASC-CM was then transferred to a 10 L Stedim Bag and 10 mM EDTA was added to prevent metalloproteases.

### ASC-CM Concentration and Diafiltration by TFF

Next, ASC-CM is concentrated and diafiltered by TFF. TFF is performed using an about 5 kD filter cutoff. As demonstrated below, the use of Tris-EDTA buffer is critical for maintaining the integrity and separation of the thousands of proteins and miRNA present in the ASC-CM.

Additional purification steps may also be taken to further concentrate the solution. Moreover, Sartobind Q filtration can be used to remove DNA into 25 mM Tris+ 10 mM EDTA pH 8.0.

Two 5 kD TangenX 0.1 m² cassettes (XP005A01L) were used for tangential flow filtration (TFF). A 3 L glass spinner outfitted with 3-port and 2-port side arms was utilized for the reservoir. One of the ports of each side-arm terminated in a diptube. The diptube on the two-port sidearm was utilized as the recirculation line and the other port terminated in a HEPA. The diptube on the 3-port sidearm was utilized for sampling and removing concentrated product. The other 2 ports on the 3-port side arm were used for the retentate line and for the feed line from the pooled ASC-CM bag.

The system was assembled 2 days prior to use and was stored in 0.01 M NaOH, after sanitization with 0.5 N NaOH for >30 min. The TFF system was rinsed to pH neutral with 1 L of sWFI (to remove any NaOH).

Next, the TFF system was conditioned using 1 L SF Medium through the feed port. The 8.5 L bag of ASC-CM was welded onto the TFF system at the feed port, and one L of concentrate was maintained in the reservoir during the initial concentration process.

The recirculation pump was started and kept at 1200 mL/min when the permeate line was opened. Permeate flow rates were maintained around 40 mL/min until the ASC-CM bag was depleted and reservoir had 1 L concentrate. At this point the pump was stopped and approximately half of the system volume (˜500 mL) was drained into the attached 1 L Erlenmeyer flask. The Erlenmeyer was then sealed and placed into the fridge.

Diafiltration into TE Buffer pH 8.0

The 3 L Tris-EDTA (25 mM Tris 1 mM EDTA, pH 8; TE) bag was welded onto the feed port. TE diafiltration was initiated and the concentrate level in the reservoir was maintained at 500 ml. When approximately 100 mL of TE remained in the bag, the TE bag was clamped off and the ASC-CM was further concentrated to ˜325 mL and removed from the system. The remaining TE was then added to the system and circulated at 200 mL/min for ˜5 min to rinse, then pooled in the Erlenmeyer flask (final volume=466 mL).

Diafiltration into Histidine Buffer pH 8.0

In an alternative embodiment, the 1 L Erlenmeyer containing 500 ml of the concentrated ASC-CM (that was removed before TE diafiltration and stored at 4° C.) and 3 L bag of 25 mM Histidine pH 8.0 (His) was then attached to the reservoir. Diafiltration into His was initiated and the concentrate level in the reservoir was maintained at 500 ml.

Permeate flow rate was maintained at 35 mL/min for the duration of the process. ASC-CM was further concentrated down to ˜350ml and removed into an Erlenmeyer flask. TFF system was rinsed with ˜100 mL of His buffer as for the TE buffer and pooled into the Erlenmeyer flask (463 mL final volume).

### Lyophilization

Next, the ASC-CM is lyophilized to form lyophilized compositions. Lyophilization cycle must be very slow and conservative in order for cake to form.

### Vial Prep and Loading

Bulk solutions were thawed at 2° C. to 8° C. Once the bulk solutions were thawed, the containers were pooled together. Sucrose, NF, was added to each formulation at a concentration of 25 mg/ml.

Schott Scc/20 mm (Part No. 68000318) tubing vials were filled to a target fill volume 2 ml, and Schott 10 cc/20 mm (Part No. 68000320) tubing vials were filled to a target fill of volume of 4 ml. Volume was verified by weight, assuming a density of 1.00 g/ml. West 20 mm V10-F597W (Part No. 19700033) stoppers were partially inserted into the vials.

Thermocouples were placed in the bottom center of eight vials. Two bottomless trays containing the product were placed on the shelves of a Hull Model 8FS12 pilot sized lyophilizer, and the tray bottoms were removed. After loading the product, the chamber was evacuated to 12 psia.

### Lyophilization

The shelves were chilled for loading and then the shelves were controlled at a target setpoint of 5° C. (±3° C.) to equilibrate the product temperatures in the vials. The shelves were then shelved at an average controlled rate of 30° C. per hour to a target setpoint of −50° C. (±3° C.) and control at −50° C. to complete the freezing step. The condenser was chilled to below −40° C. and the chamber was evacuated to the target pressure of 40 microns (±10 microns).

Chamber pressure was controlled at the target setpoint of 40 microns by bleeding in 0.2 μm filtered Nitrogen, NF into the chamber.

The shelves were warmed to a target setpoint of −38° C. (±3° C.) at an average controlled rate of 15° C. per hour and control at the setpoint to complete primary drying. The shelves were next warmed to a target setpoint of 20° C. (±3° C.) at an average controlled rate of 15° C. per hour and control at the setpoint for secondary drying to reduce the residual moisture content.

The chamber was backfilled with 0.2 μm filtered Nitrogen, NF to atmospheric pressure, and the vials were stoppered and unloaded from the chamber.

### Results

### Cell Culture:

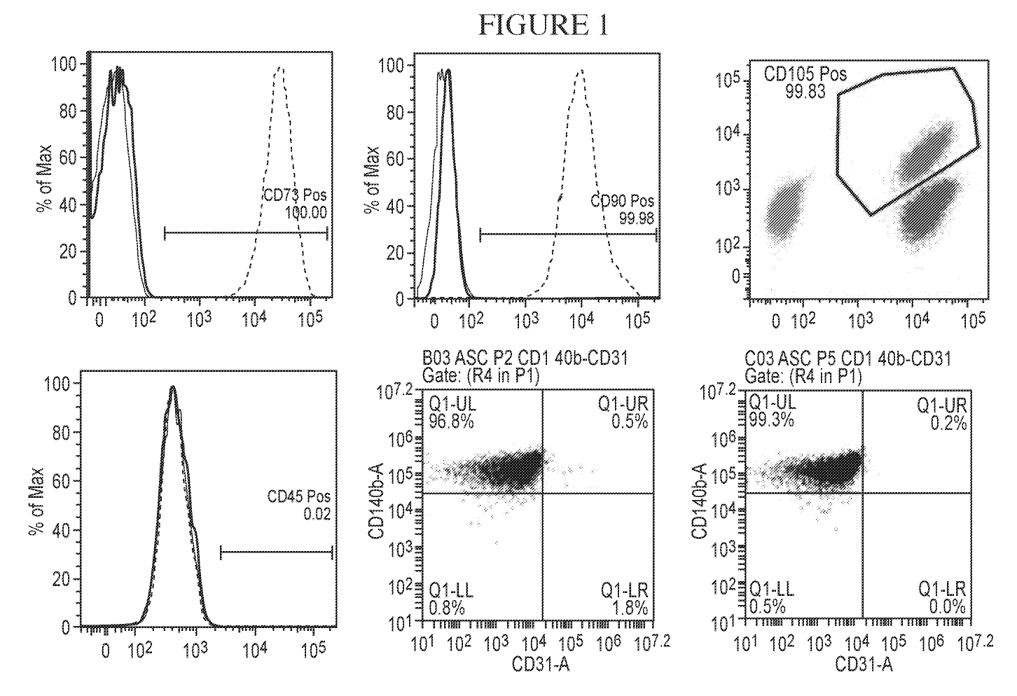

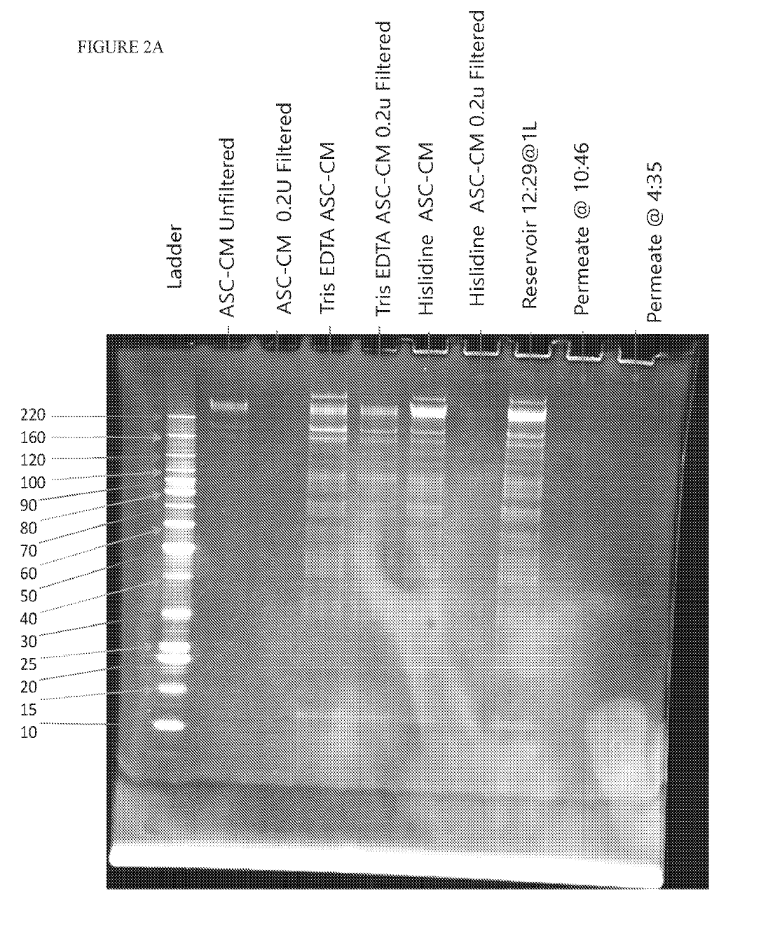

ASC express classical mesenchymal markers. Flow cytometric analysis of Cell Care Therapeutics ADSC express MSC markers of CD73, 90, 105 and are negative for CD45. Data is shown from one human donor in FIG. 1. Also shown in the bottom two figures is that ADSC express the CD140b pericytic marker and are negative for the endothelial marker CD31 at p2 through p5.

### Filtration:

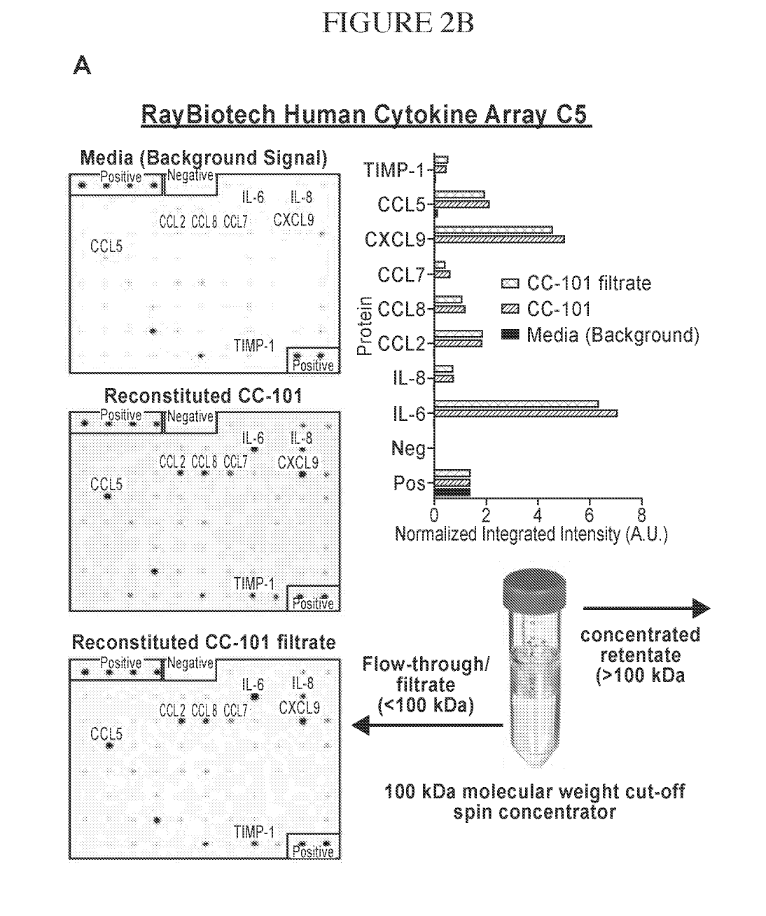

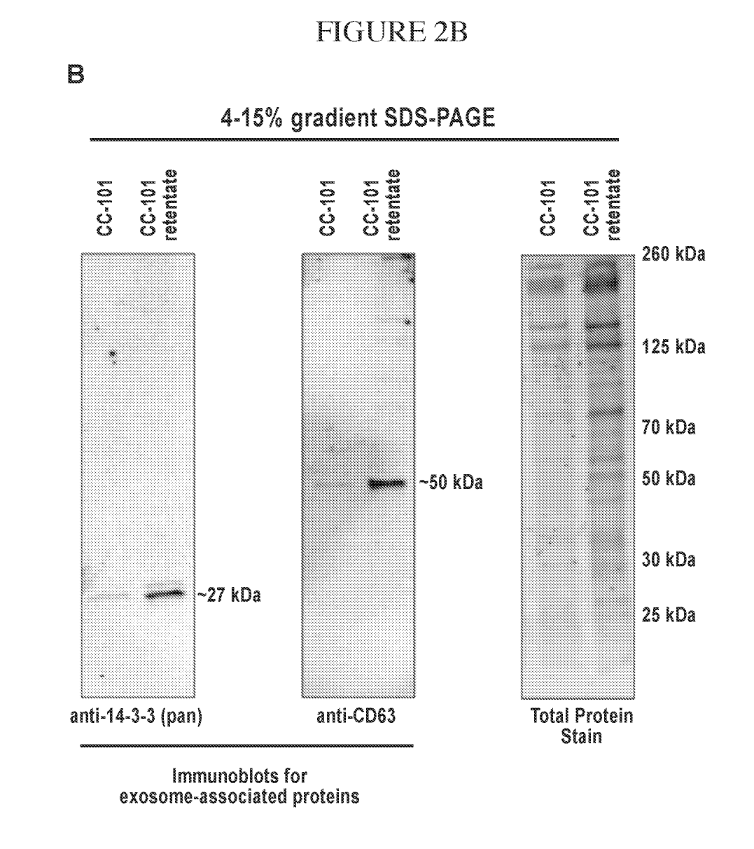

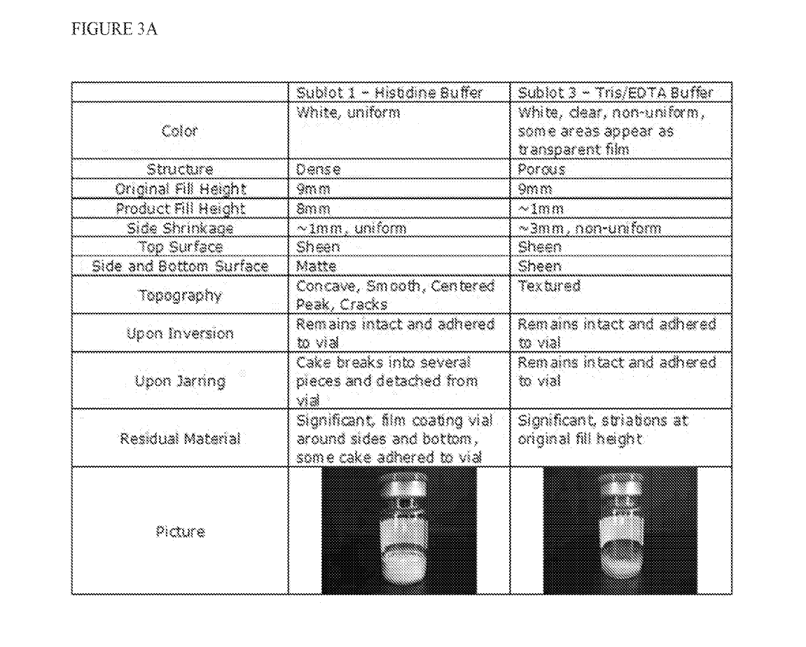

Effective secretome recovery and purification following TFF and diafiltration observed with SDS-PAGE/Filter are shown in FIG. 2A.

### Lyophilization:

The Histidine formulation appeared slightly hazy with large particles floating around in the solution (definitely not just undissolved sucrose) with a pH of 7.4. The Tris/EDTA formulation appears clear and colorless with a pH of 8.

Based on the results of Freeze Drying Microscopy (FDM), the Tris/EDTA formulation needs to be maintained below −46° C. for Primary Drying, which is very low. Thus, the Tris/EDTA formulation requires a very conservative cycle.

The Histidine formulation performed a little better and needs to be maintained below ˜30° C. for Primary Drying.

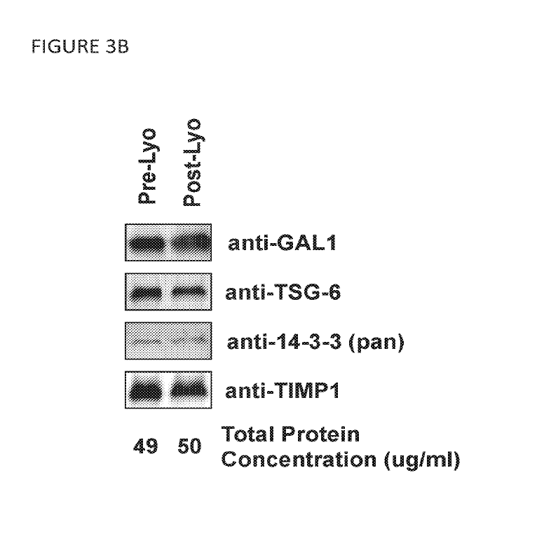

The results of the physical inspection following are shown in FIG. 3A.

### Reconstitution and pH:

| | Reconstitution | Description of Constituted | |
|---|---|---|---|
| Sublot | Time (seconds) | Solution | pH |
| 1 - Histidine | 20, 20, 20 | All three vials appeared | 7.3, 7.1, |
| Buffer | | clear and colorless | 7.1 |
| 2 - Tris/EDTA | 60, 80, 40 | All three vials appeared | 7.8, 7.8, |
| Buffer | | clear and colorless | 7.8 |

### HT-DSC (High Temperature Differential Scanning Calorimetry):

| Sublot | Scan Rate | Event |
|---|---|---|
| 1 - Histidine | 2° C./min | Glass Transition at 85.6° C. |
| 1 - Histidine | 10° C./min | Glass Transition at 78.7° C. |

HT-DSC could not be performed on Sublot 3 because material was very hard and sticky.

### TGA (Thermogravimetric Moisture Analysis):

| Sublot | Scan Rate | Weight Loss Event | Onset of Degradation |
|---|---|---|---|
| 1 - Histidine | 10° C./min | 2.4% | 173.1° C. |

TGA could not be performed on Sublot 3 because material was very hard and sticky.

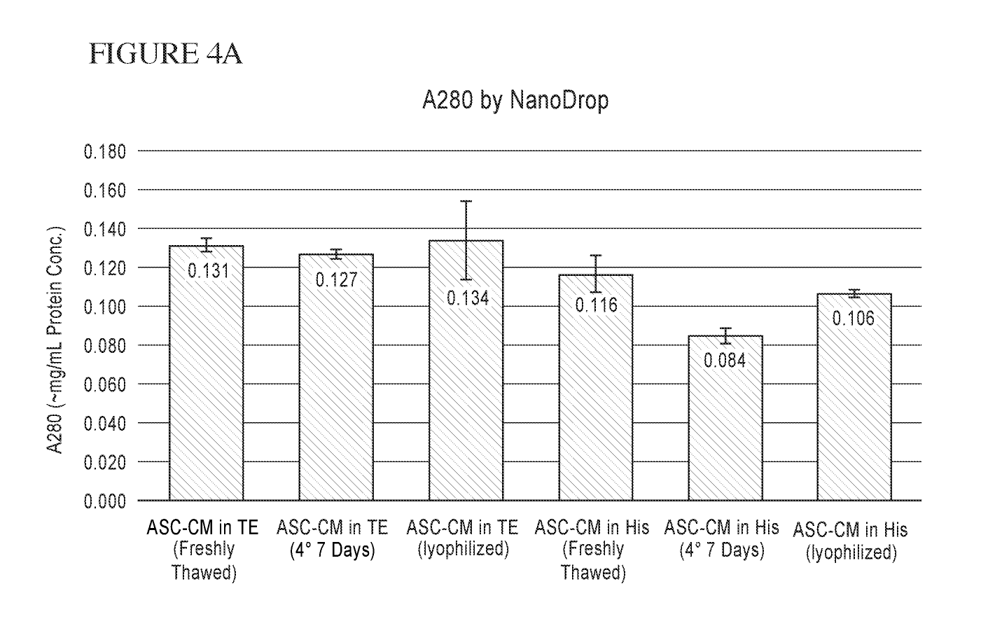

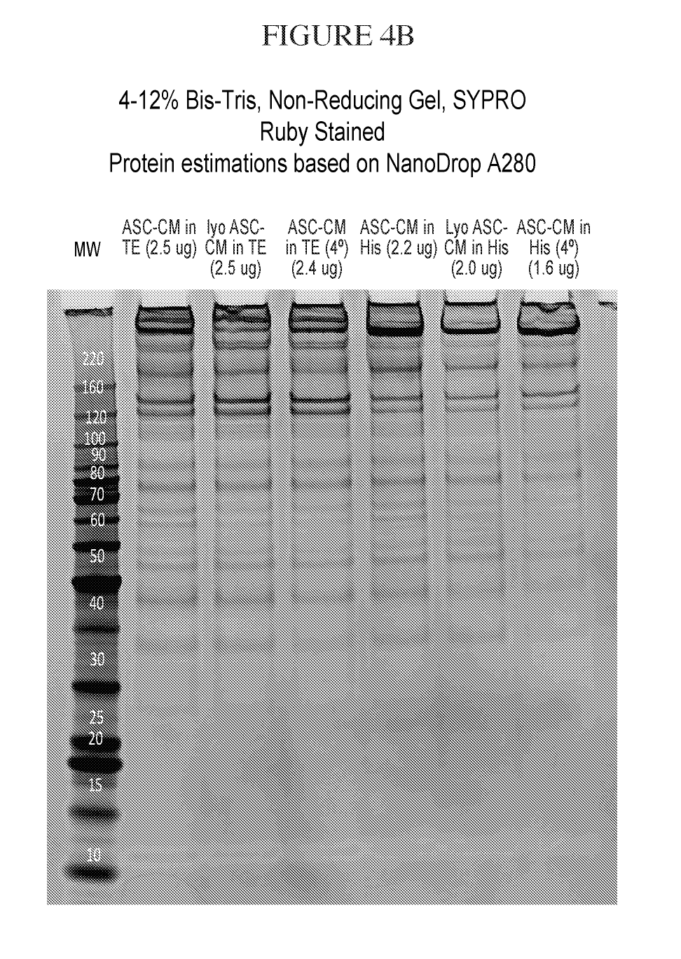

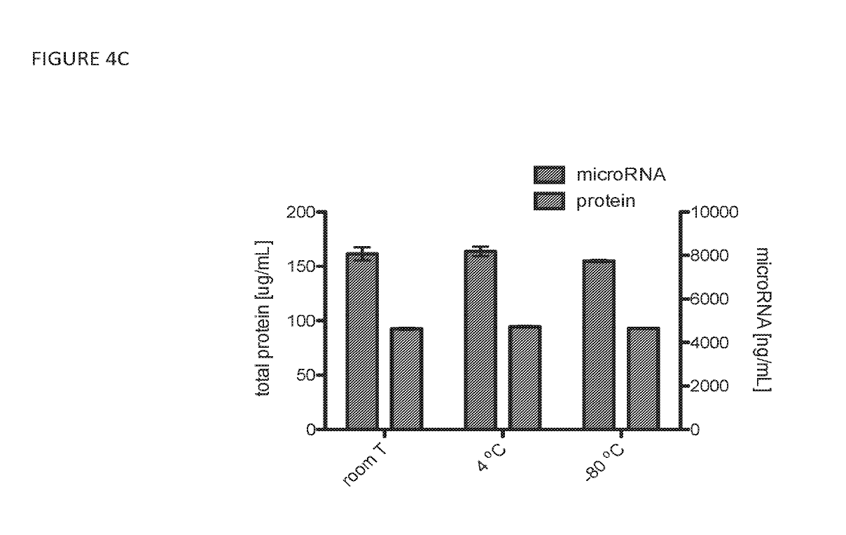

Product stability maintained following lyophilization. No significant difference in protein concentration or band profile was observed between the pre- and post-lyophilization TE and HIS samples. (See FIGS. 4A and 4B).

Protein and miRNA Content:

The results of Qubit protein concentration (μg/ml) are summarized in the table below. (Samples at 4° C. for >week).

| Sample | Protein [μg/mL] | microRNA [ng/mL] |
|---|---|---|
| ASC-CM unfiltered | 6.5 +/− 2 | 523.5 +/− 14 |
| Post-TFF ASC-CM-TE | 47.1 +/− 3 | 4415 +/− 65 |
| Post-Lyo ASC-CM-TE | 42.5 +/− 6 | 3900 +/− 50 |
| Post-TFF ASC-CM-His | 54.3 +/− 4 | 3980 +/− 80 |
| Post-Lyo ASC-CM-His | 32 +/− 6 | 1770 +/− 10 |

Protein and microRNA concentrations are average of 3 measurements. Data obtained using Qubit total protein kit and microRNA Assay kit.

Stability of the lyophilized CC-101 was also studied under different storage temperatures, and total protein and total miRNA were not significantly affected. Lyophilized CC-101 was stored at room temperature, 4° C. or −80° C. for 21 days. Following incubation, samples were dissolved in 1 mL of H₂O. Total protein and microRNA concentration were measured in triplicate using Qubit Protein Assay and Qubit microRNA kit respectively. The results are shown in FIG. 4C.

### DNA Removal/Sartobind Q:

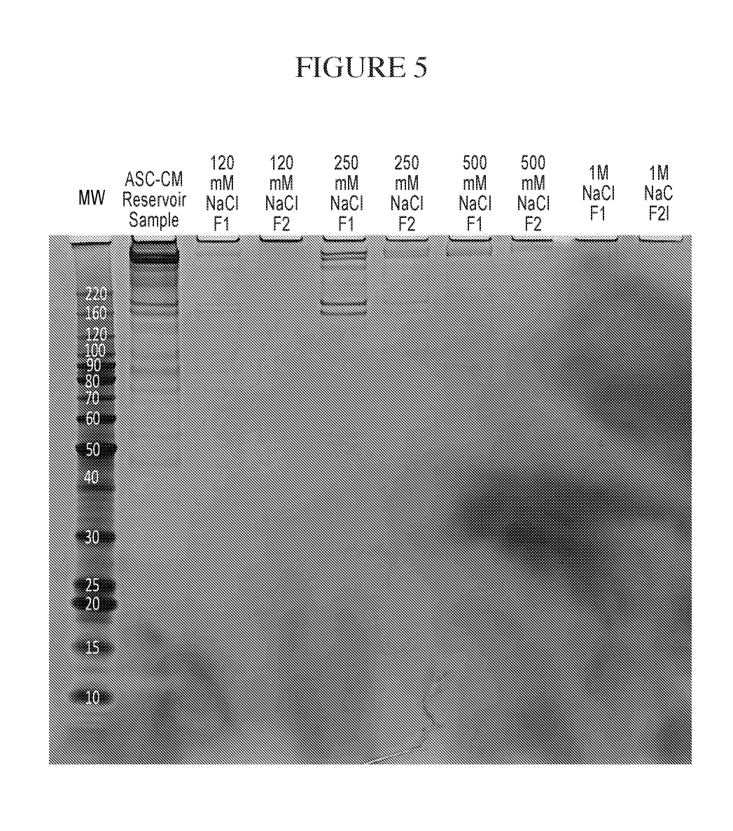

DNA Estimation by Picogreen showed that ASC-CM had 2 mg DNA, while the reservoir had 1.35 mg. Post Sartobind Q DNA Elution using 1M NaCl resulted in 0.11 μg/ml DNA being eluted. No DNA was eluted at concentrations lower than 1M NaCl. It is desirable to run ASC-CM through Sartobind Q Column and/or washing with a suitable volume of 500 mM NaCl before proceeding with TFF. The results are shown in FIG. 5.

### CFSE Immunopotency Assay:

This assay is designed to assess the degree to which each ASC cells (p5 or post induction harvested p5 ASC) or ASC-CM sample (either intact; post TFF, or post lyophilization) can suppress the proliferation of T helper (CD4+) lymphocytes. Samples (or ASC cells) were tested using cryopreserved leukocytes purified from the peripheral blood of healthy individuals.

The IPA measures the suppression of CD4+ T-cell proliferation via flow cytometry using the tracking dye Carboxyfluorescein Diacetate, Succinimidyl Ester (CFSE) in conjunction with anti-human CD4 fluorescently labeled antibody.

Briefly, primary Peripheral Blood Mononuclear Cells (PBMCs) were stained with CFSE and cultured according to manufacturer's protocol. Labeled cells were plated at 4×10⁵ leukocytes per well (1×10⁶ cells/mL density) containing the either the ASCs as above or treated with ASC samples (CM or post TFF, or post lyophilization). This results in titrated PBMC:ASC cells or equivalent volume of samples (CM or post TFF, or post lyophilization) ratios of 1:1, 1:0.5, 1:0.2, 1:0.1, and 1:0.05.

Additional wells were plated with stimulated PBMCs alone, and 1:0.05 ratio of PBMC: ASC cells or equivalent volume of samples (CM or post TFF, or post lyophilization) without stimulation, all which serve as controls. Bone marrow (BM) MSC cells were plated in the same ratio against PBMC (resulting suppression by BM-MSC serves as the reference immunosuppression for the assay).

The PBMC alone control serves as the positive control for maximum T cell proliferation against which the degree of ASC (or equivalent volume of CM or post TFF, or post lyophilization)-mediated suppression was measured. The non-stimulated 1:0.05 ratio well was used to generate a negative control gate against which proliferation is measured. Concomitantly, T cell-stimulatory monoclonal antibodies, anti-human CD3 and anti-human CD28 were added to each well.

Cells were cultured for 4 days at 37° C.; collected and stained with anti-human CD4-fluorescent antibody, anti-human CD14 fluorescent antibody and live/dead stain. Upon staining, cells were collected analyzed for proliferation via CFSE intensity of CD4+ [CD14⁻ 7AAD⁻] cells using flow cytometry.

The results of the immunopotency assay (expressed in normalized IC50) are summarized in the table below.

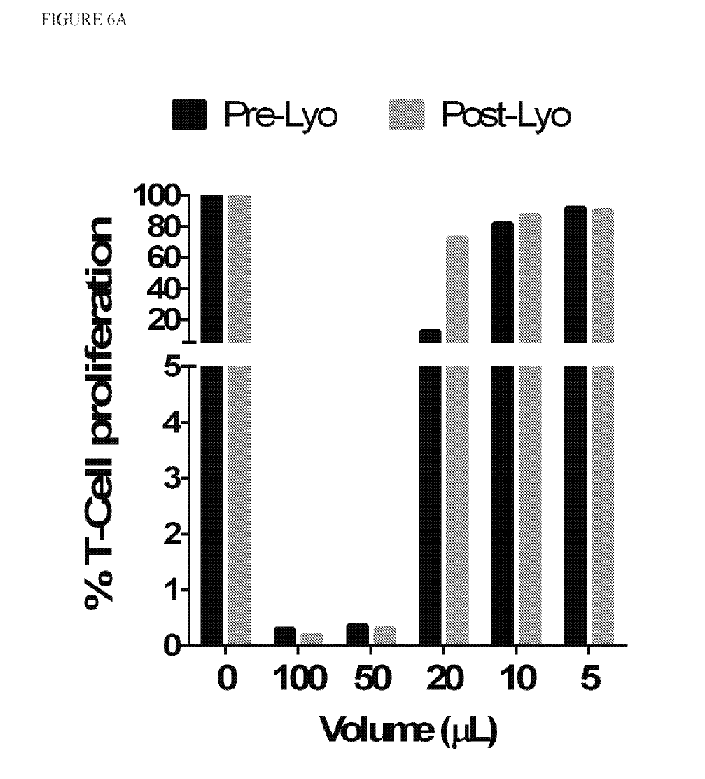

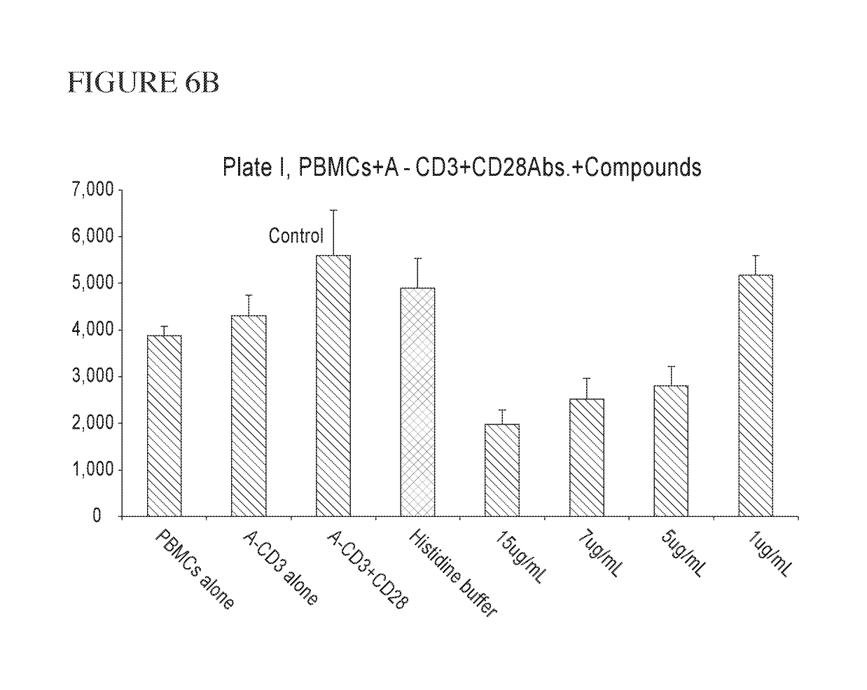

Note a significant decrease in T-cell proliferation in a dose dependent fashion when equal amounts of T-cells were plated with increasing dose of ADSC-CM. The data is shown in FIG. 6A.

### BRDU Immunopotency Assay:

T-cell suppression was assessed using time-resolved fluoroimmunoassay based on the incorporation of BrdU (5-bromo-2′-deoxyuridine) into newly synthesized DNA strands of proliferating cells. When cells are cultured with labeling medium that contains BrdU, this pyrimidine analog is incorporated in place of thymidine into the newly synthesized DNA. Incorporated BrdU is detected using a europium labeled monoclonal antibody.

### Addition of Cells to Assay Plates (Day-0)

PBMCs (isolated from heparinized human whole blood by Ficoll/Hypaque, density gradient centrifugation) were plated into 96-well round bottom plate as follows: The cells are resuspended in culture medium at a concentration of 1×10⁶cells/mL and 100 μL (100,000 cells/well) of this solution is added to each well of the plate. The total volume in each well is made to 200 μL with culture medium. Only the internal sixty wells are used in each assay. The culture plates are incubated in a humidified incubator at 37° C. for 72-96 hours. For assay wells in which the responder cells are stimulated soluble anti-CD3 and anti-CD28 Abs at 5 μg/mL and 2 μg/mL, respectively

Each experimental condition was set up in four or five replicates to measure cell proliferation. In each assay, additional controls are also set up including 10 replicate wells of PBMC cells stimulated with only anti-CD3 Ab, (maximum/positive stimulation control) and 5 replicates of PBMC cells cultured in the absence of both anti-CD3 and anti-CD28 Ab (no/negative stimulation).

### Addition of Drug Compound (Day-0)

Post TFF ASC-CM His induced sample (in histidine buffer) (CC-101) was added at four defined concentrations in a volume of 50 μL to each well in replicates of five wells for each assay condition and 25 mM Histidine buffer as a control. Cultures (assay plates) are incubated for four days in an incubator at 37° C. with 5% CO2

### Addition of Eu Labeled BrDu (Day-3)

At the end of 72 hours, the cells are pulsed with Eu++ (Europium) labeled BrDu which is added to each well (20 μL/well) and plates incubated for additional 16 to 18 hours in a humidified incubator at 37° C.

### Harvest of Assay Plates (Day-4)

Next day the labeled cells are harvested and processed as per the Delfia cell proliferation protocol and T cell proliferation is measured using time resolved fluorescence (non- radioactive) method. The stimulation and proliferation of PBMC cells is reflected in the measure Europium counts (FIG. 6B)

The measured data is calculated in two different ways following determination of the mean values (e.g., from quadruplicates wells) of each experimental condition.

In one scenario data is expressed as a standard stimulation index (SI) that is defined as the mean of experimental wells divided by the mean of the control wells (unstimulated). In another method data is expressed as net counts or cpm (cpm experimental—cpm background/unstimulated).

### ASC-CM Contains Exosome and Non-Exosome Associated Proteins:

The results of an experiment to separate ASC-CM into fractions with and without exosomes are shown in FIG. 2B. Approximately 95% of the volume of reconstituted CC-101 was filtered using a 100 kDa molecular weight cut-off spin concentrator in which biological products (e.g., proteins or protein complexes) smaller than 100 kDa flow through the filter (filtrate) while biological products greater than 100 kDa (e.g., proteins, protein complexes or exosomes) are concentrated in the retentate. Note that cytokines in the filtrate can be detected on membrane-based antibody arrays comparing expression levels of many cytokines/chemokines. Quantification of antibody array spot intensities shows similar abundance of the cytokines present in the pre and post-filtered CC-101. Culture medium was used as a control for nonspecific background signal. The assay was performed according to the manufacturer's (RayBiotech) instructions for use with a LI-COR Odyssey infrared imaging system. The results indicate that the detected cytokines do not appreciably associate with exosomes or in higher molecular weight complexes that would be restricted for passage across the filter membrane. SDS-PAGE and immunoblot analyses of the unfiltered, reconstituted CC-101 and the concentrated retentate show that 14-3-3, a protein incorporated into exosomes, and CD63, a tetraspanin incorporated into the lipid membrane of exosomes are enriched in the retentate despite their individual molecular weights below the 100 kDa when resolved under the exosome disrupting conditions of SDS-PAGE. For immunoblot analysis of proteins, samples were combined with 4X SDS-SB. Samples were boiled, subjected to SDS-PAGE using standard methods, and transferred to immobilon-FL PVDF membrane (EMD Millipore, Billerica, Mass.) using standard electrotransfer methods. Membranes were blocked with LI-COR blocking buffer and probed with primary antibodies to the proteins of interest followed by fluorescent secondary antibodies of appropriate species reactivity and fluorescence spectra (LI-COR, Lincoln, Nebr.) and imaged on an Odyssey infrared scanner (LI-COR) according to the manufacturer's instructions.

### Paracrine Factors Released by ADSC are Unaffected by the Lyophilization Procedure:

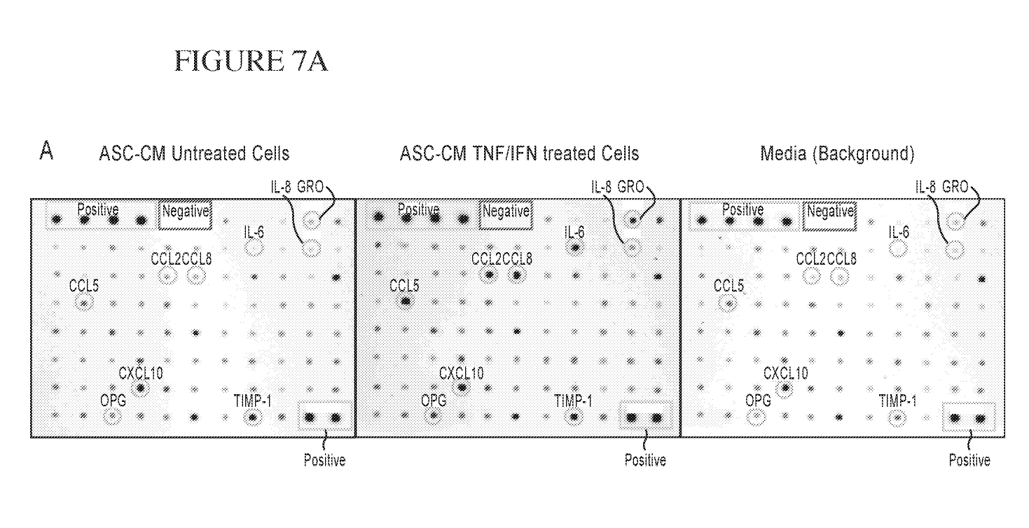

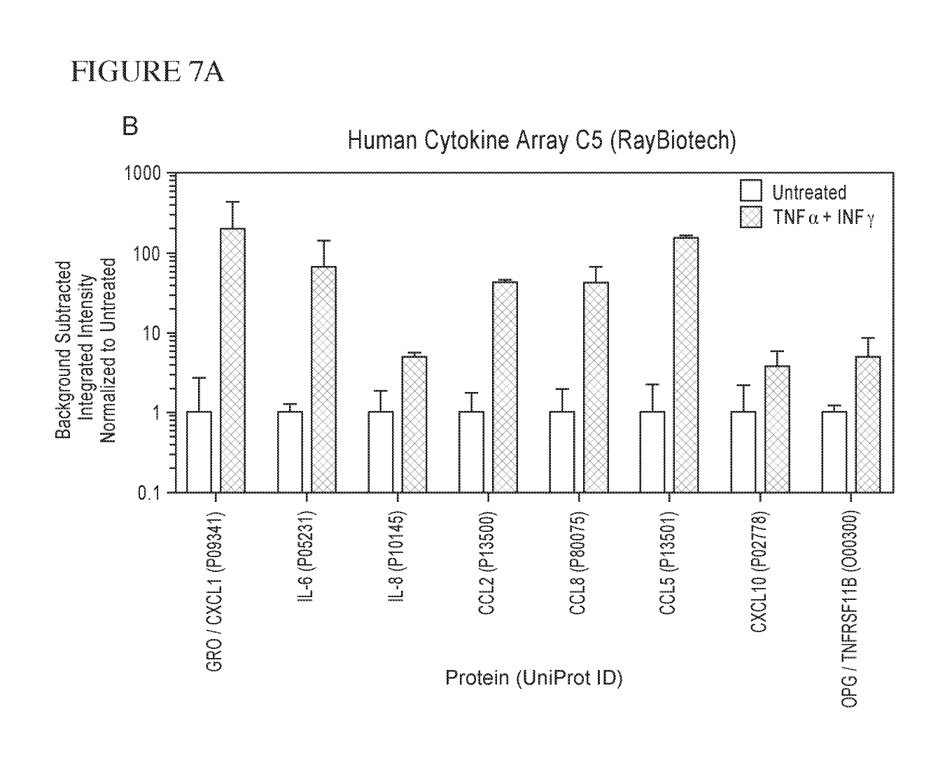

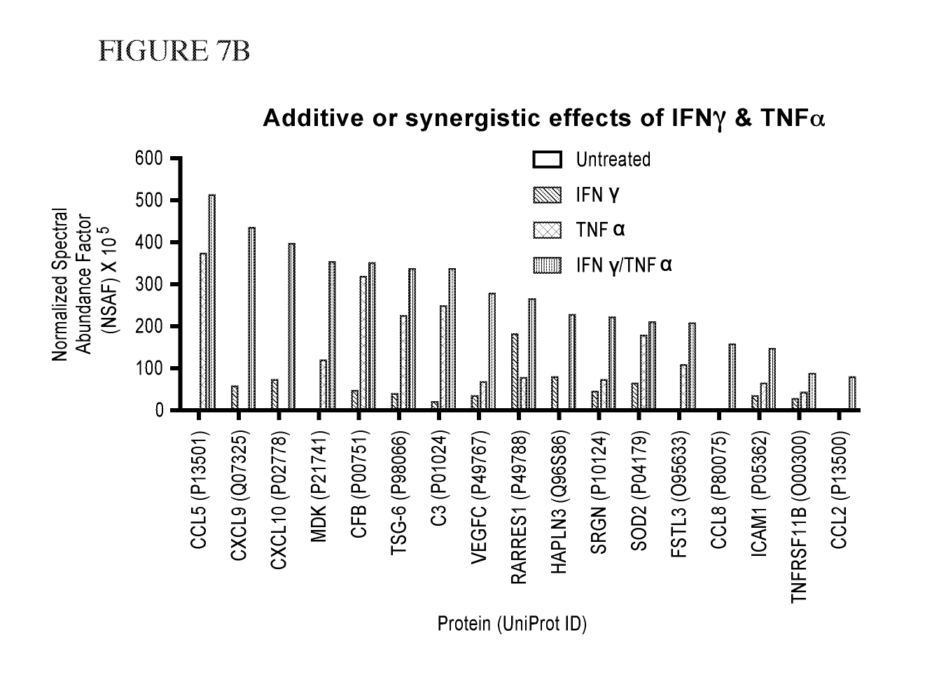

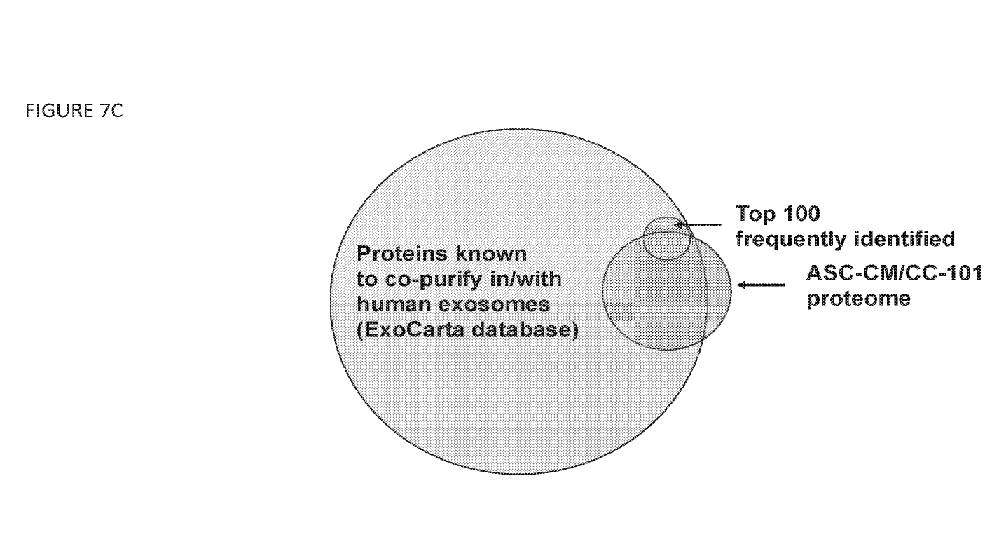

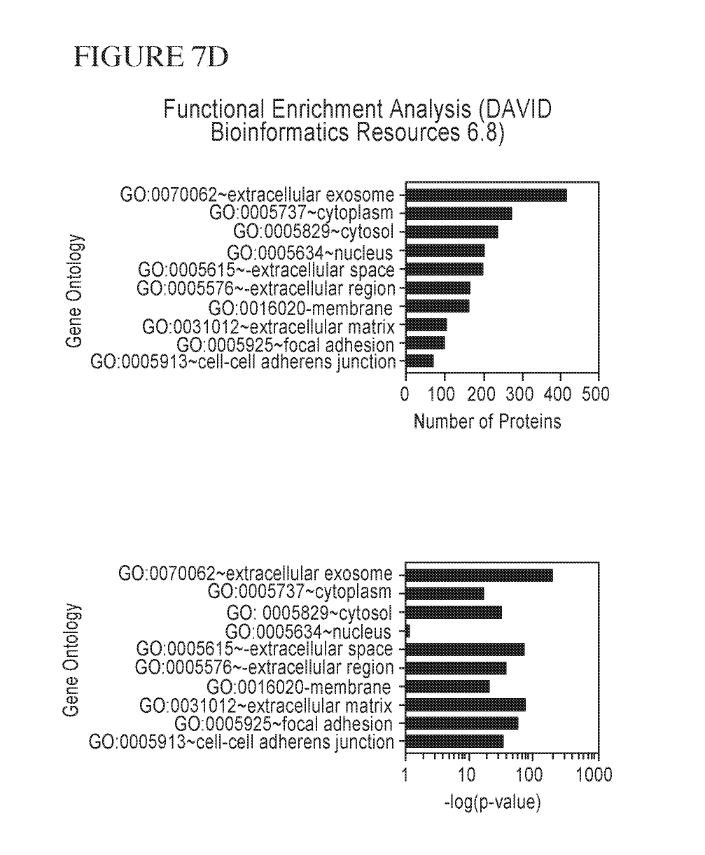

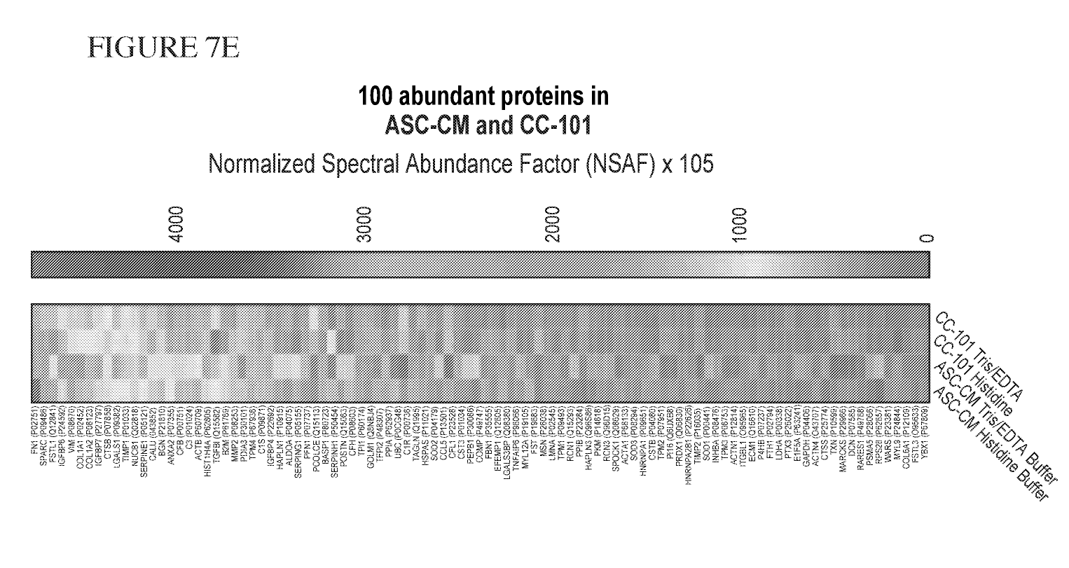

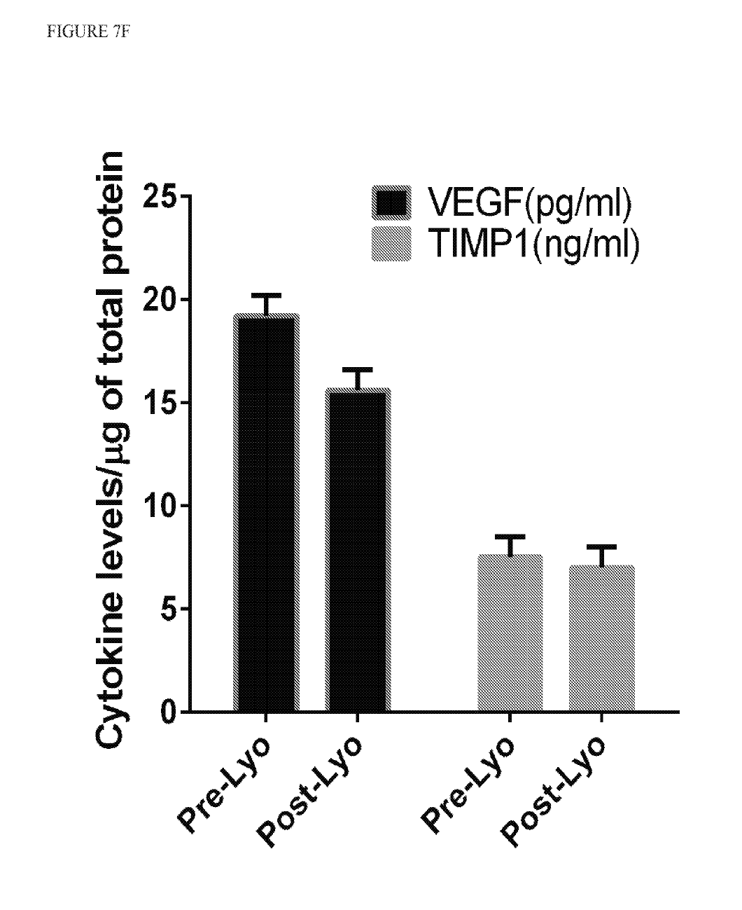

Both VEGF and TIMP1 were measured in the cell supernatants of ADSC by ELISA assays. VEGF concentrations are very low (pg/ml) and at a sub therapeutic level to drive angiogenesis. As expected, pre and post-lyophilization procedure, the amounts of VEGF and TIMP1 detected were similar. ELISA results are shown in FIG. 7F.

Relative stability of proteins pre- and post-lyophilization were also examined by immunoblot analysis. CC-101 (Post-Lyo) was resuspended to the same volume as the processed ASC-CM from which it was derived (Pre-Lyo) (FIG. 3B). Similar total protein concentrations were confirmed using Qubit Protein Assay Kit and a Qubit 3.0 fluorometer (ThermoFisher). Samples were subjected to SDS-PAGE and immunoblot analysis with antibodies to Galectin 1 (GAL1), TSG-6, 14-3-3 proteins, and TIMP1, showing similar abundance of these proteins pre and post-lyophilization. Immunoblots were performed as described above.

### Paracrine Factors Released by ADSCs are Increased by Cytokine Stimulation:

FIG. 7A and FIG. 7B show that IFNγ, TNFα or the combination of the two increases the expression of a number of proteins in ASC-CM. As shown above, membrane-based antibody arrays can be used to measure the abundance of many cytokines/chemokines at once. 7A (panel A) shows representative images of antibody arrays comparing expression levels of cytokines/chemokines in ASC-CM from cells untreated or treated with IFNγ and TNFα. Culture medium was used as a control for nonspecific background signal. The assay was performed according to the manufacturer's instructions for use with a LI-COR Odyssey infrared imaging system. Quantification of selected cytokine expression profiles indicates that IFNγ/TNFα treatment stimulates the expression of CXCL1, IL-6, IL-8, CCL2, CCL8, CCL5, CXCL10 and TNFRSF11B by at least 2-fold.

An increase in paracrine factors from cells stimulated by IFNγ and TNFα was also observed using label-free quantitive shotgun proteomic analyses. Proteins from ASC-CM were precipitated with trichloroacetic acid (TCA), washed with ice-cold acetone twice, dried, and stored at −20° C. until further processing. Protein samples were resuspended in 8M urea in 100 mM Tris pH 8.5, reduced, alkylated and digested by the sequential addition of lys-C and/or trypsin proteases. The digested peptide solution was fractionated online using strong-cation exchange and reverse phase chromatography and eluted directly into an Q Exactive mass spectrometer. MS/MS spectra were collected and subsequently analyzed using the ProLuCID and DTASelect algorithms. Database searches were performed against a human database. Protein and peptide identifications were further filtered with a false positive rate of less than 5% as estimated by a decoy database strategy. Normalized spectral abundance factor (NSAF) is calculated as the number of spectral counts (SpC) identifying a protein, divided by the protein's length (L), divided by the sum of SpC/L for all proteins in the experiment. This is a common metric for comparing relative protein abundances across experiments in label free proteomics. FIG. 7B shows that IFNγ, TNFα or the combination increases the expression of a number of paracrine factors. Moreover, IFNγ and TNFα have synergistic effects on the expression of a number of the factors including CXCL9, CXCL10, VEGFC and TSG-6.

The synergistic effect of combined TNFα and IFNγ treatment on TSG-6 was independently confirmed by immunoblot and dot-blot analysis of the ASC-CM. FIG. 8 diagrams various lengths of cytokine treatment and shows that that the combination of TNFα and IFNγ and length of stimulation increase the expression of TSG-6 within the cell (Cell Lysate) and the ASC-CM. The methods of immunoblot analyses are described above. For dot-blot analysis, samples were directly bound to immobilon-FL PVDF under vacuum suction using a Bio-Dot microfiltration apparatus. Membranes were blocked with LI-COR blocking buffer and probed with primary antibodies to the proteins of interest followed by fluorescent secondary antibodies of appropriate species reactivity and fluorescence spectra (LI-COR, Lincoln, Nebr.) and imaged on an Odyssey infrared scanner (LI-COR) according to the manufacturer's instructions. To quantify expression, the background subtracted integrated fluorescence intensities of the bands or dots were determined using LI-COR Odyssey software.

### Proteomic Analysis to Characterize the Composition of ASC-CM/CC-101:

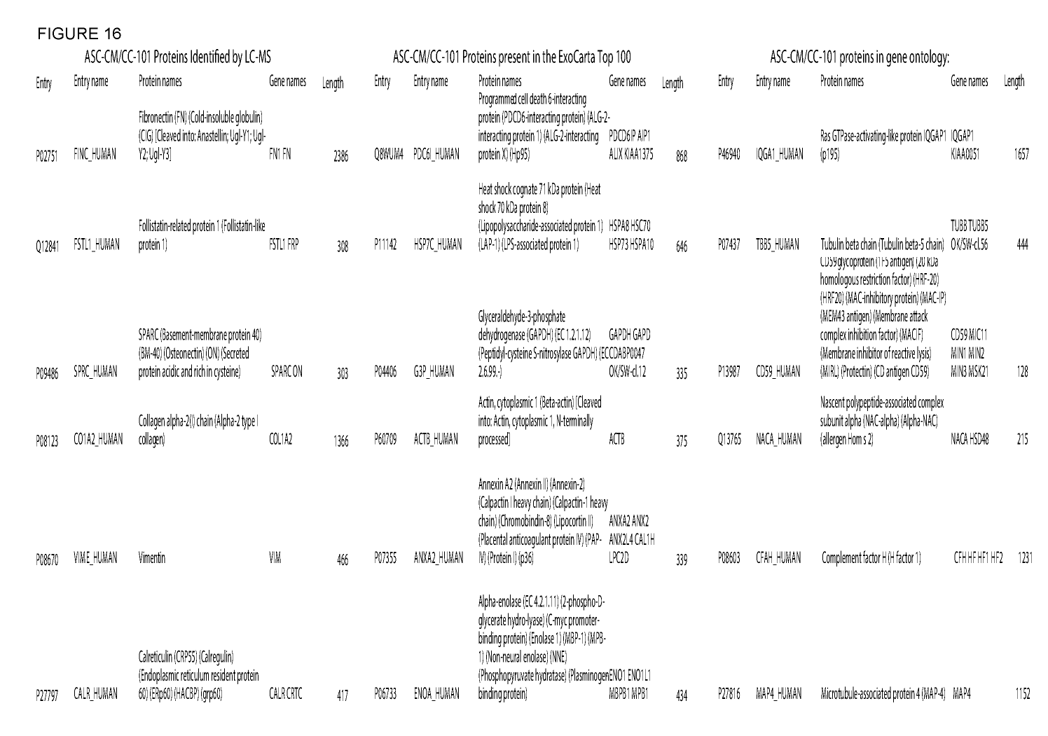

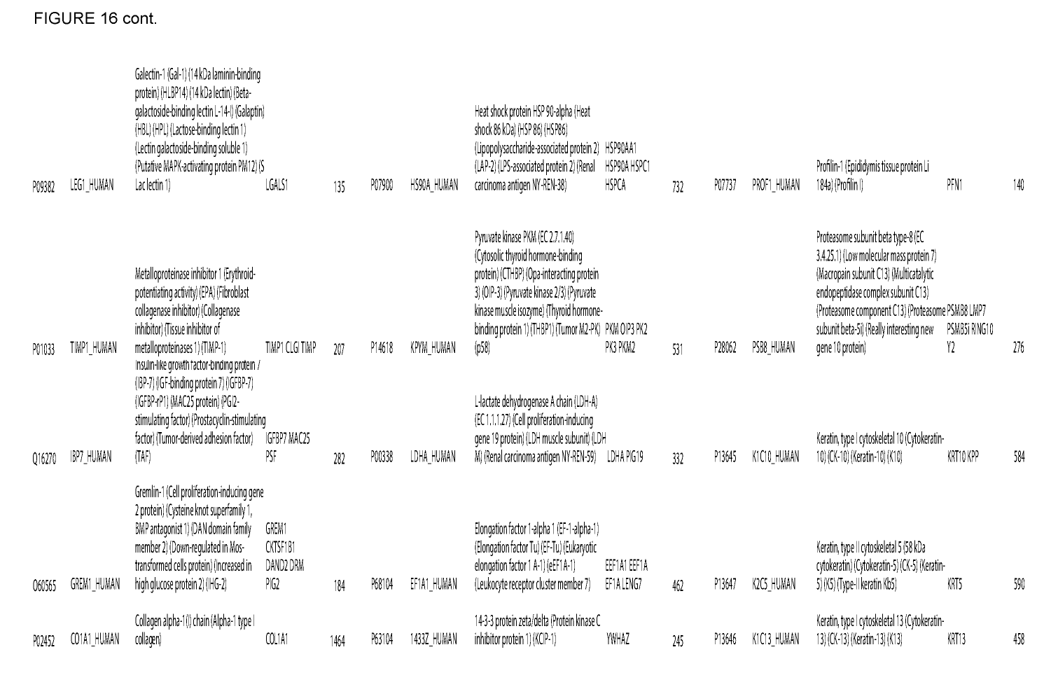

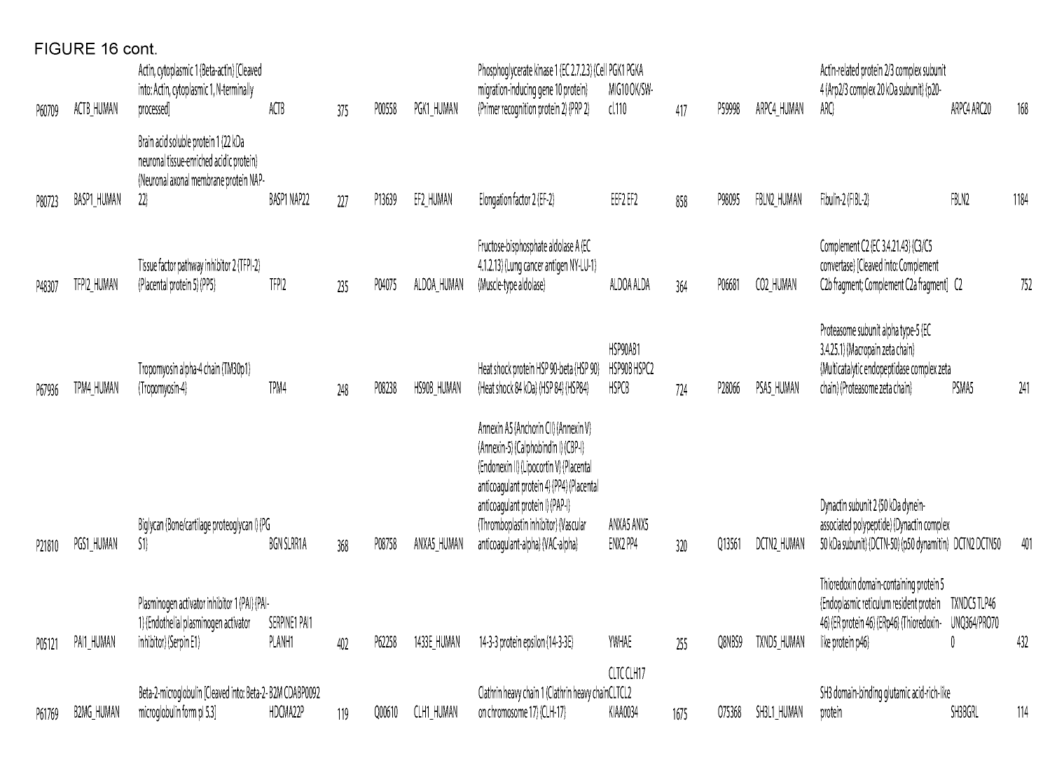

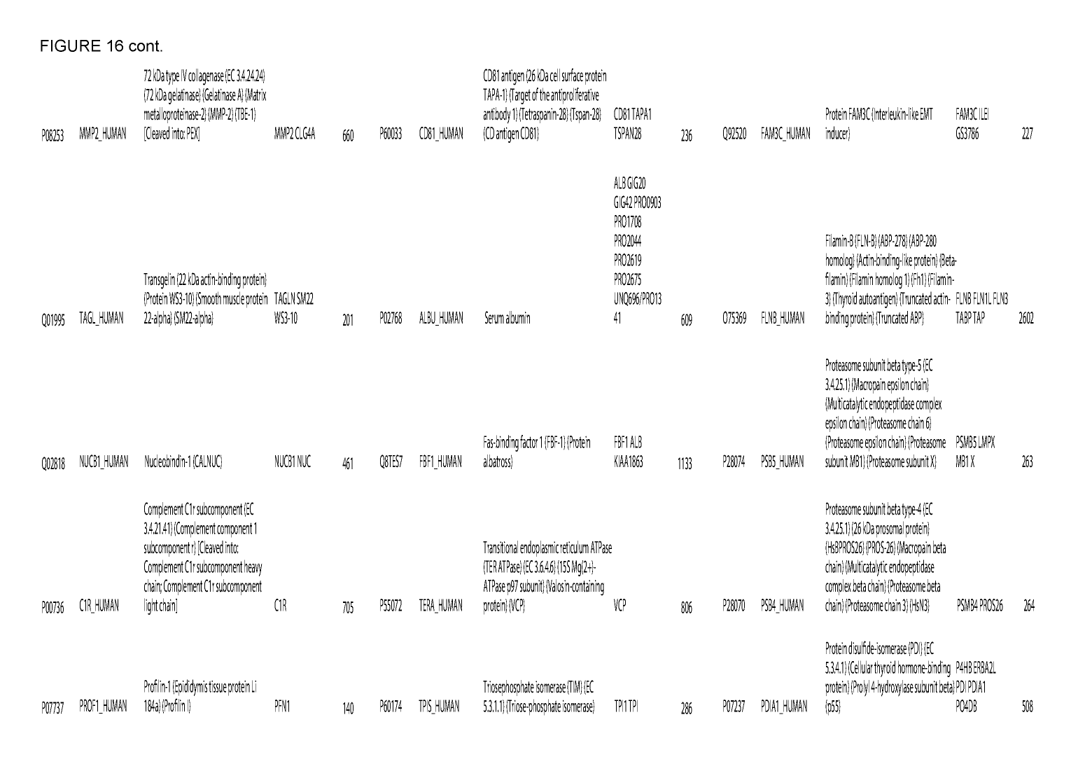

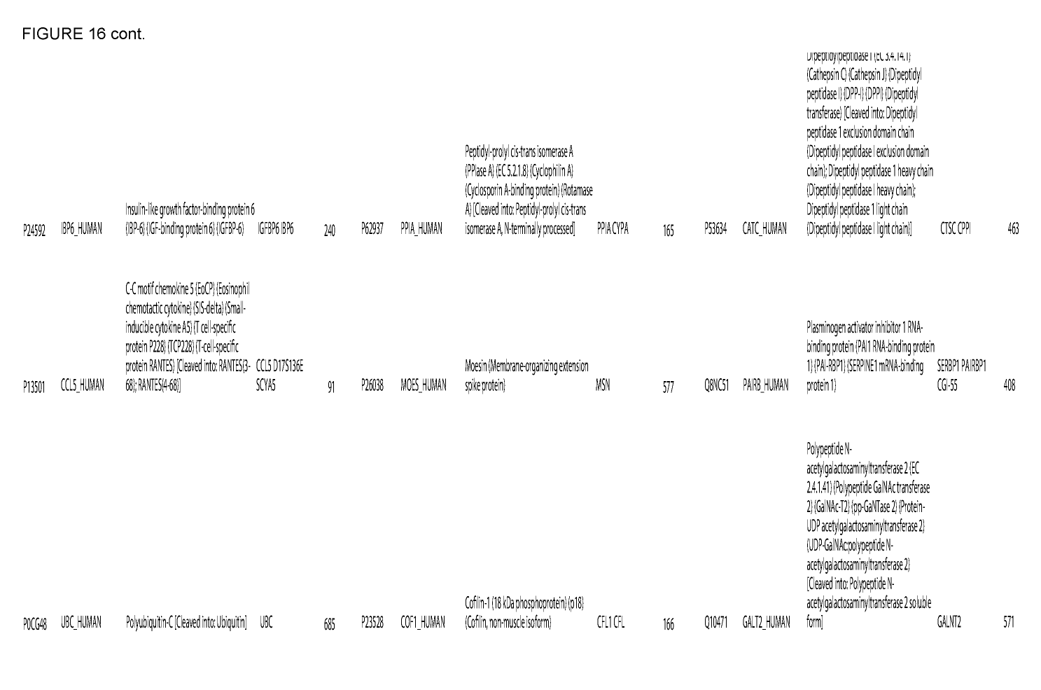

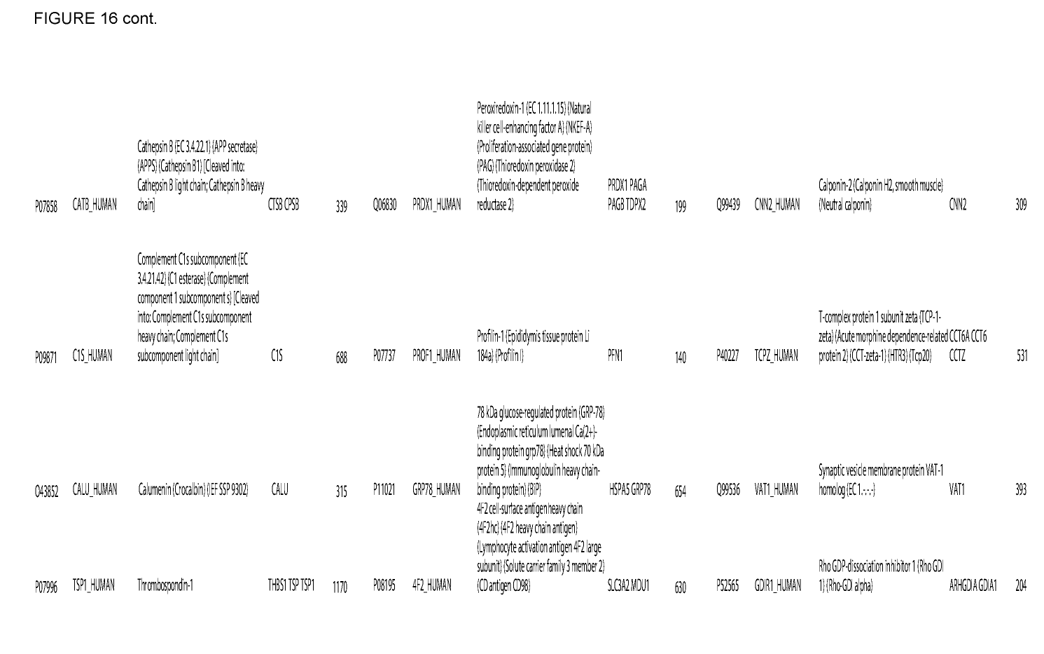

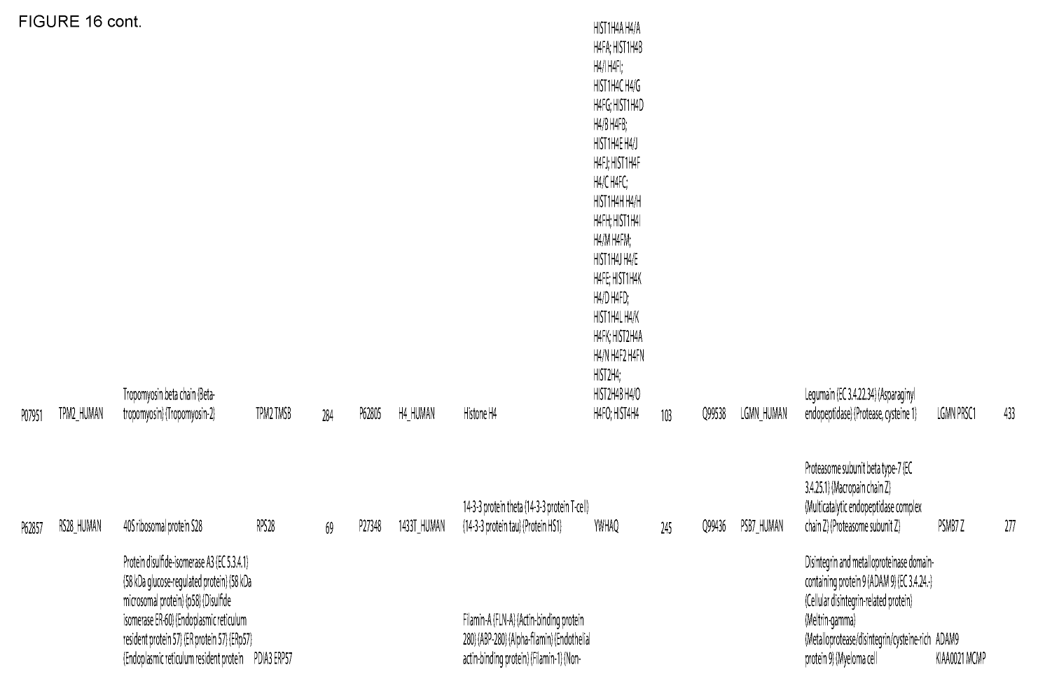

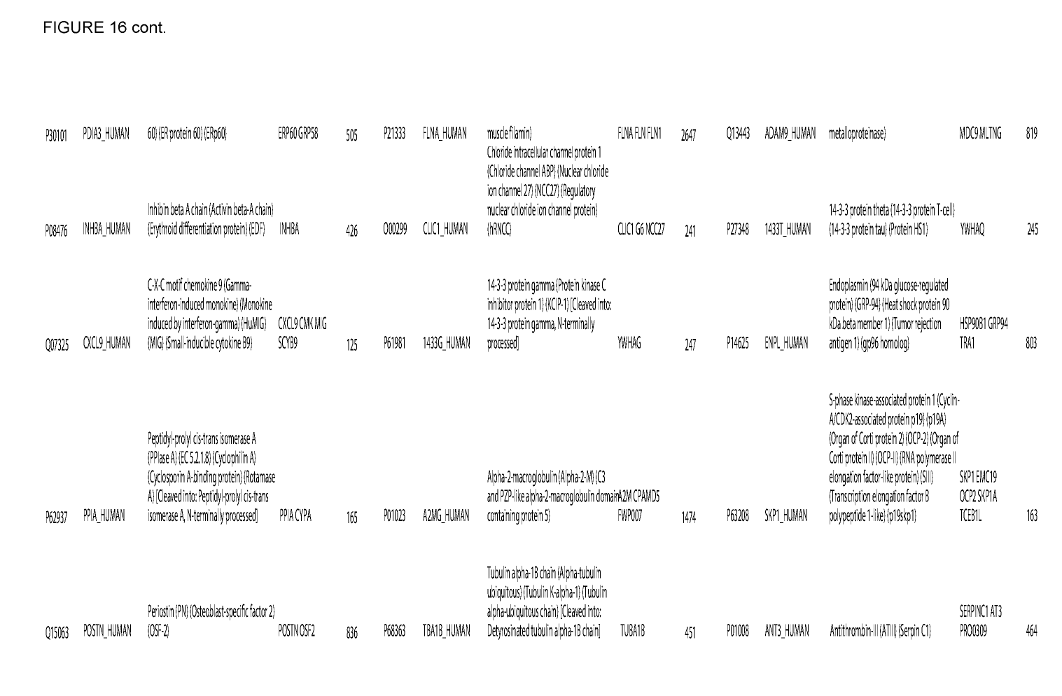

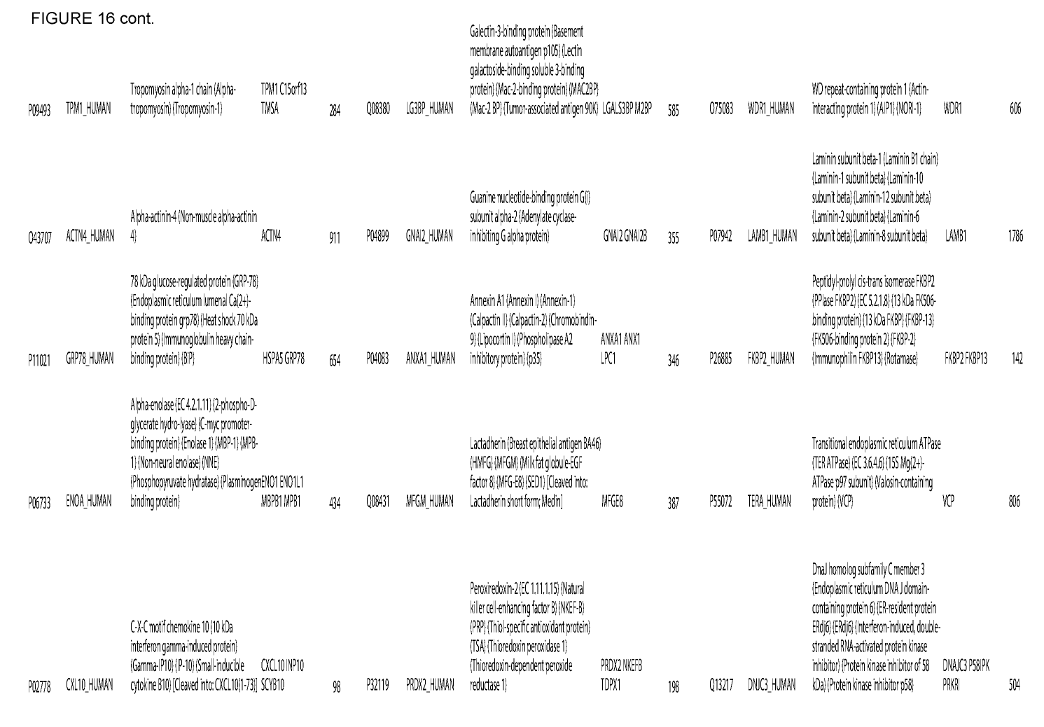

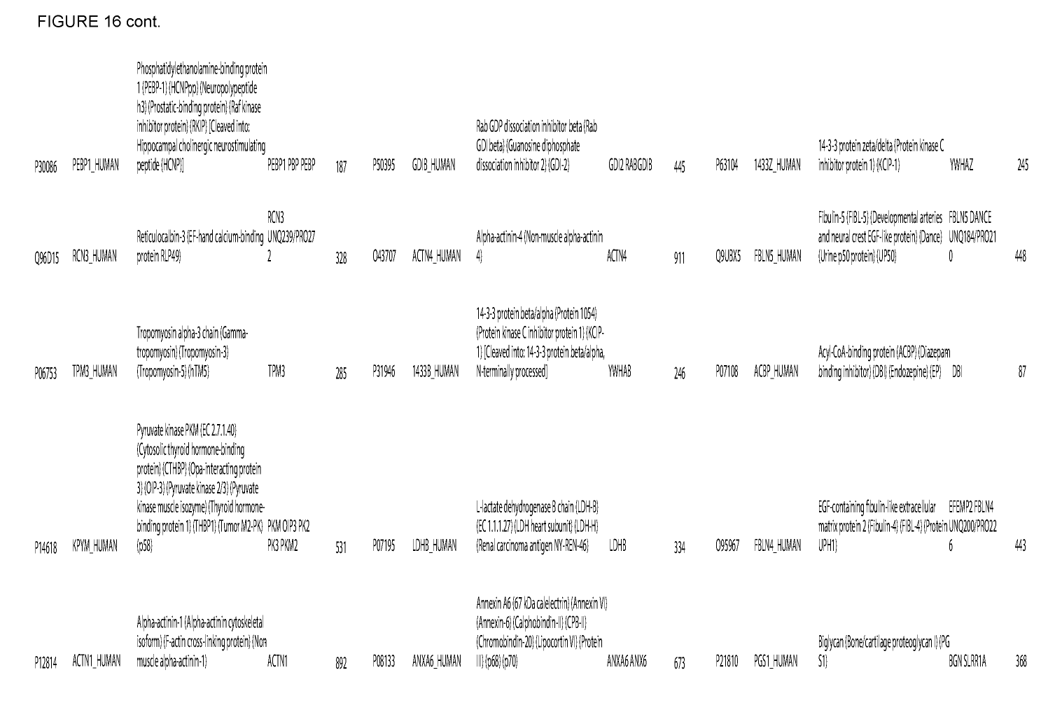

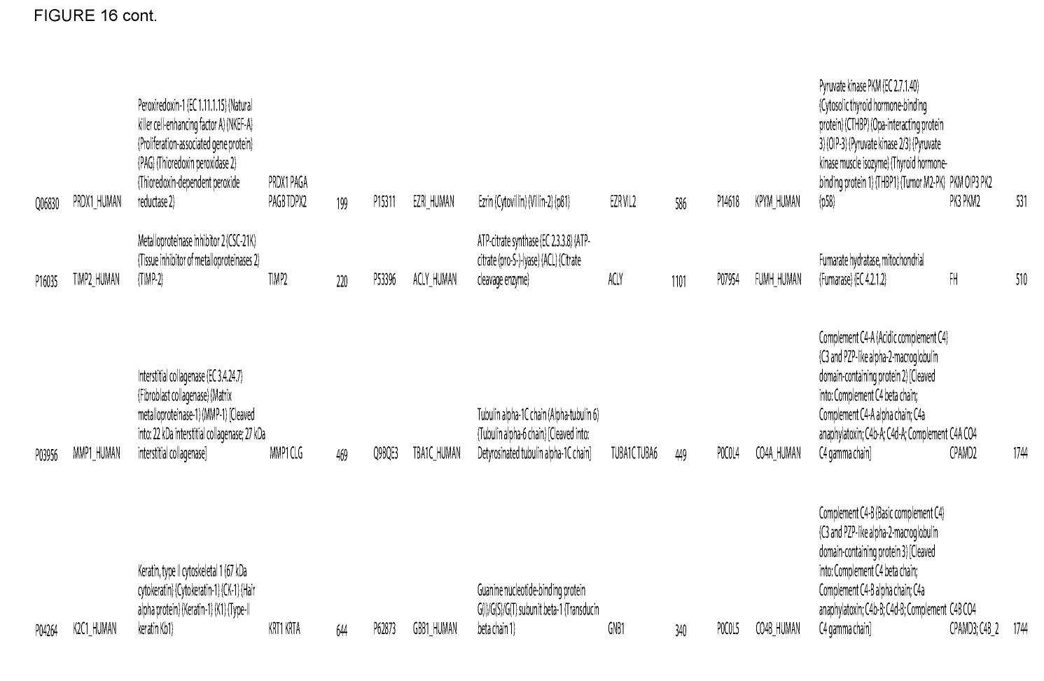

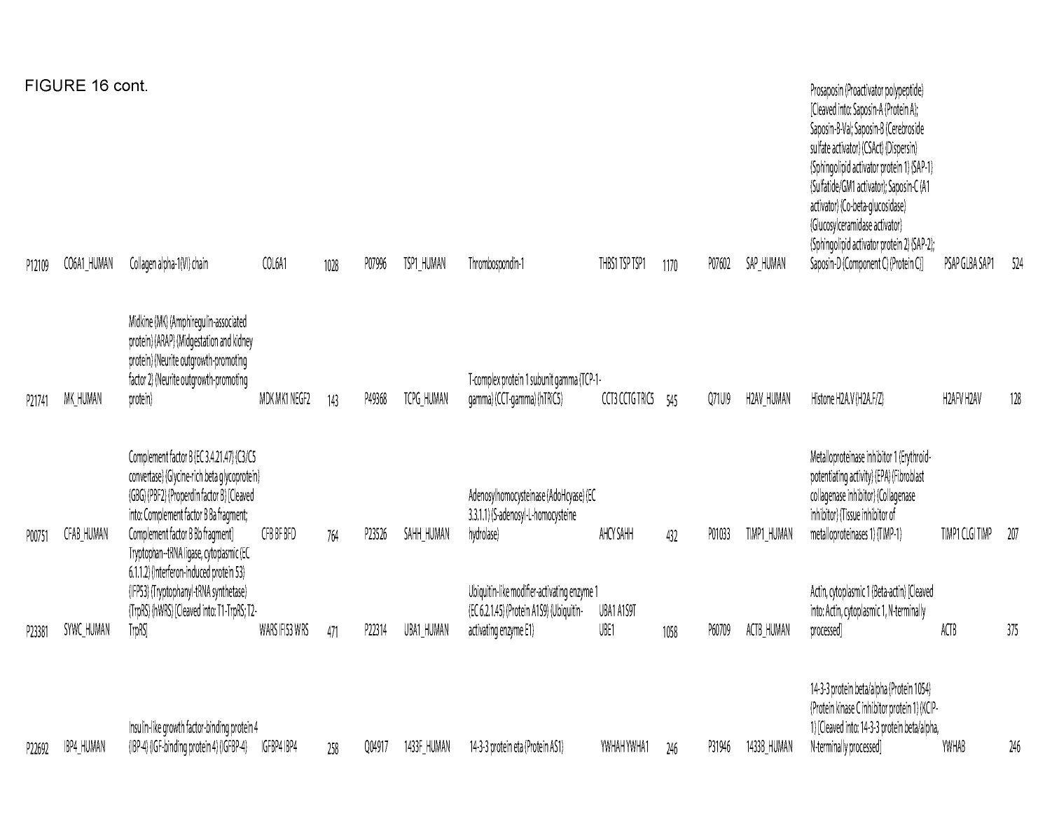

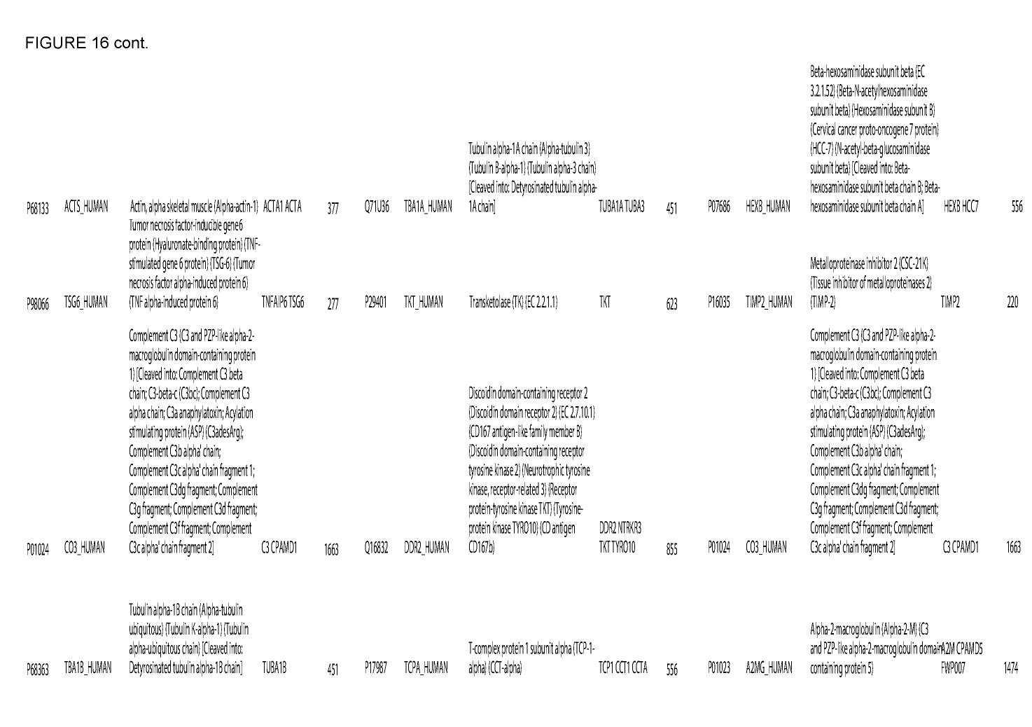

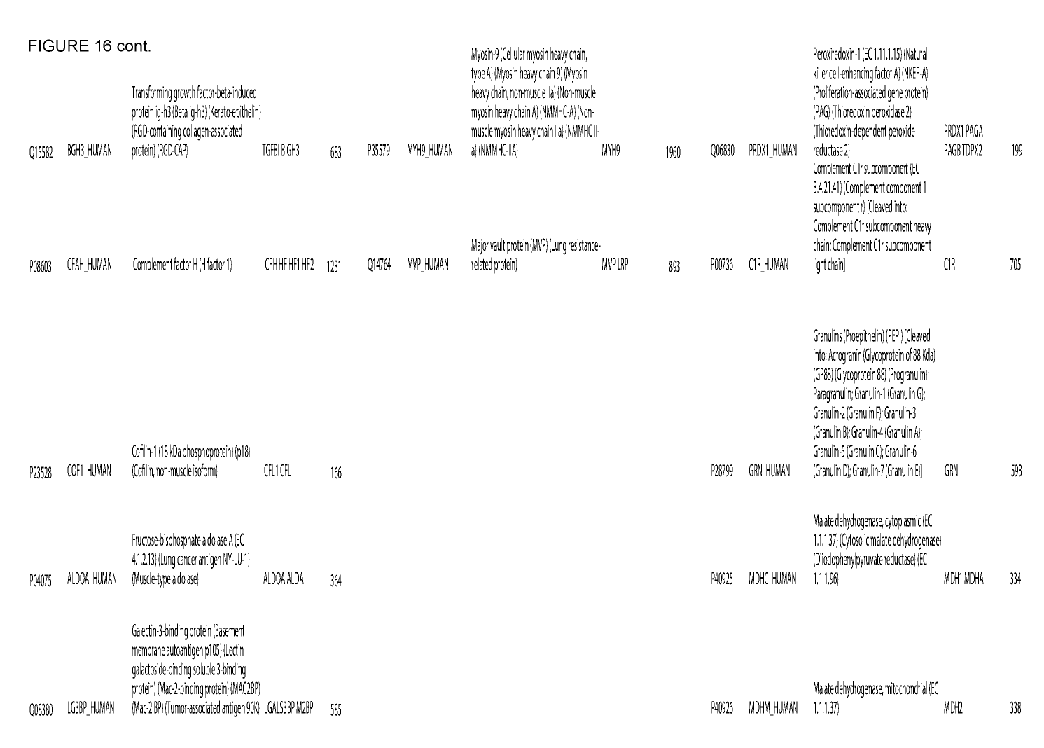

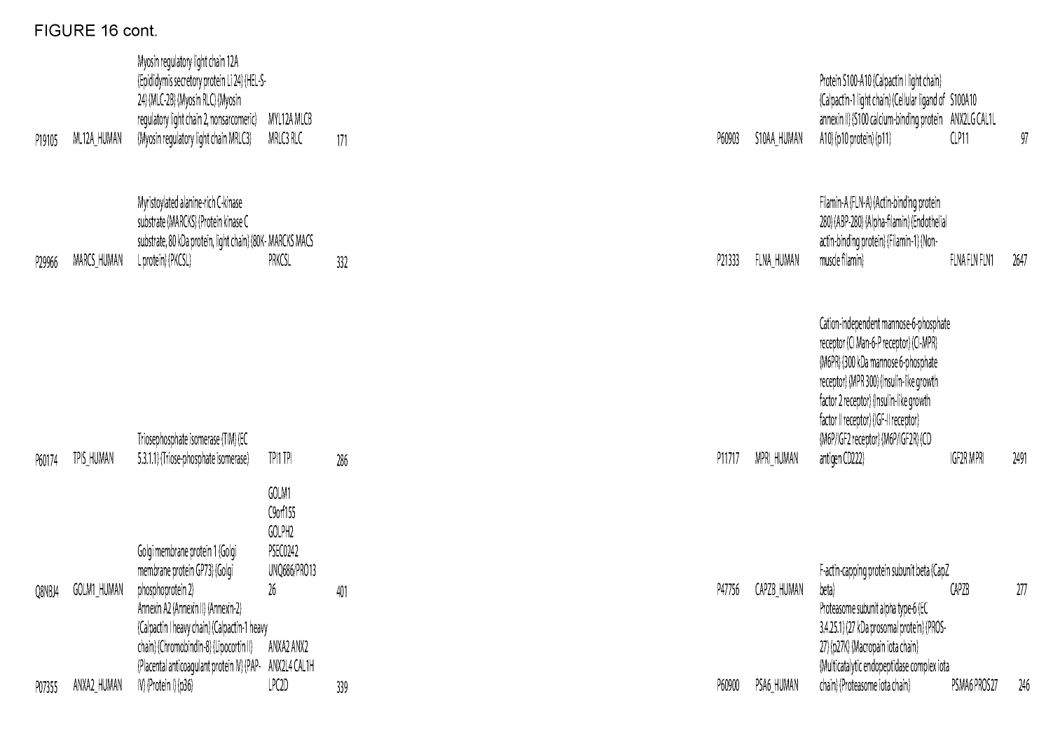

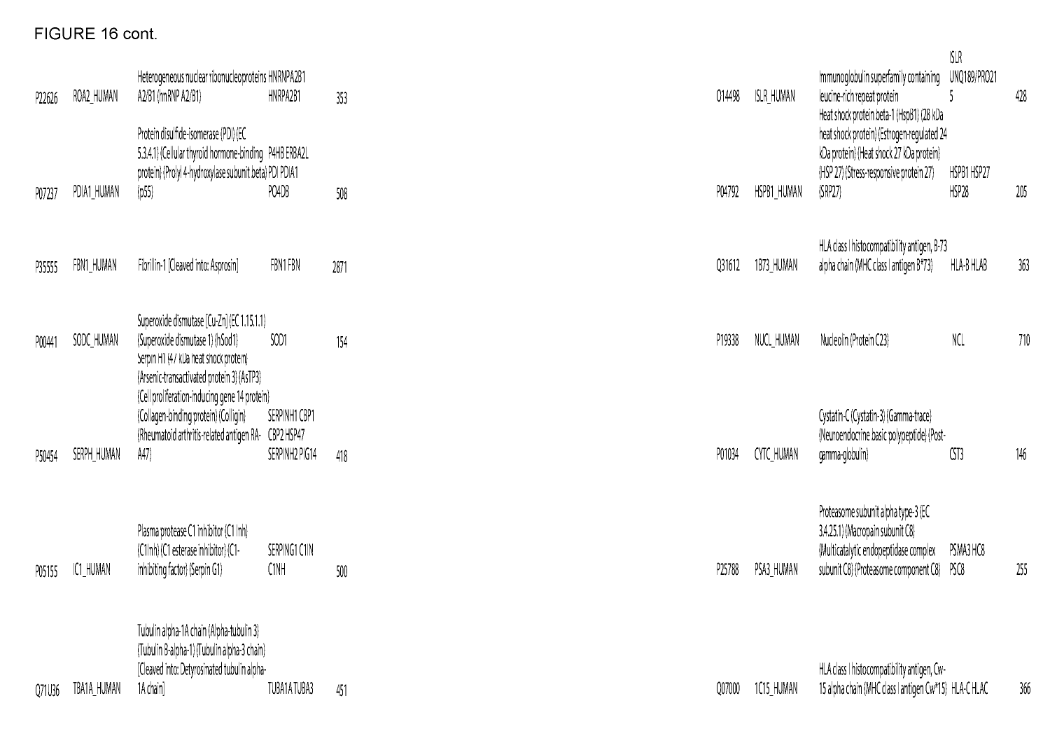

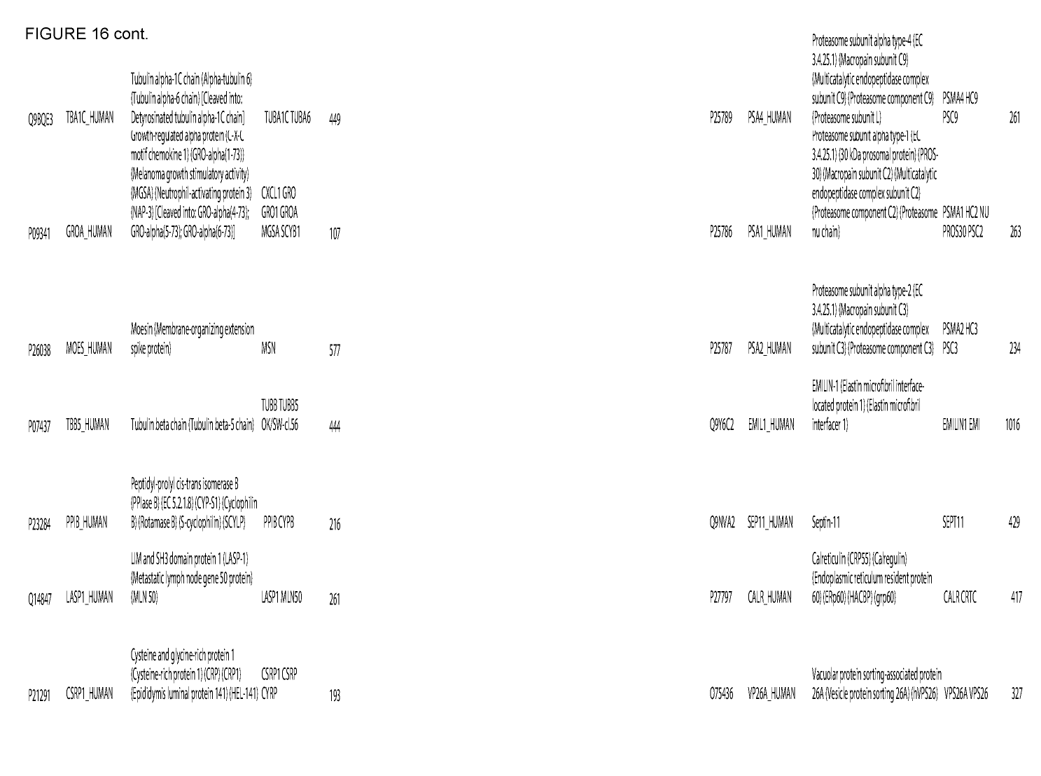

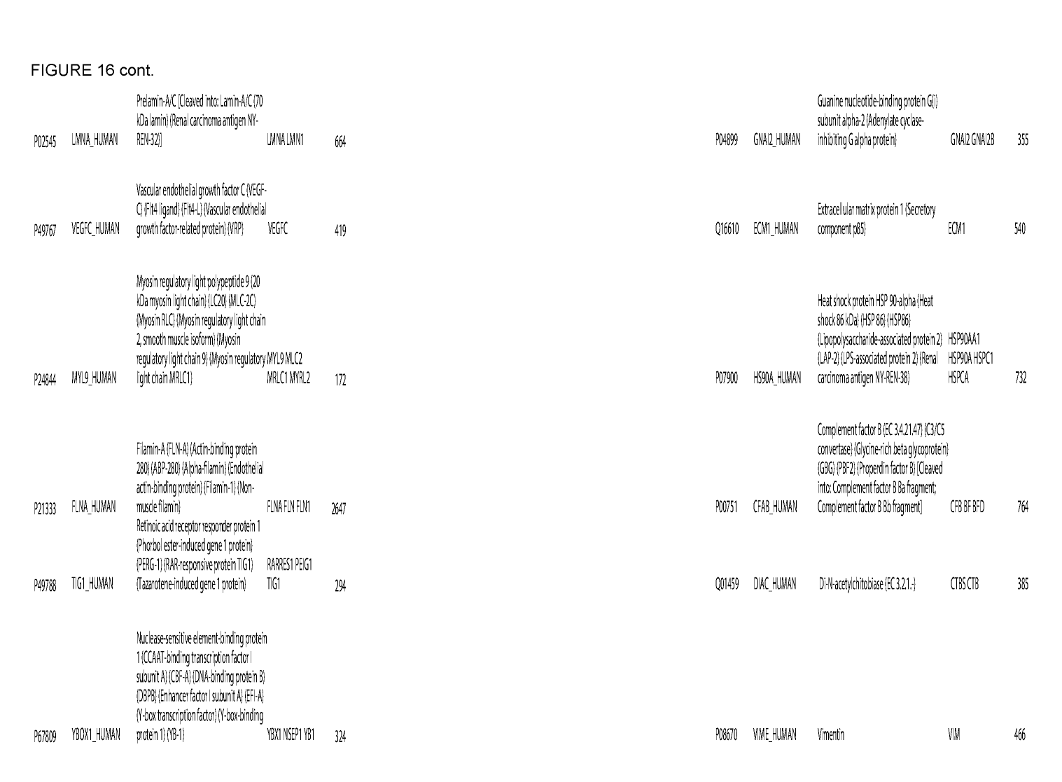

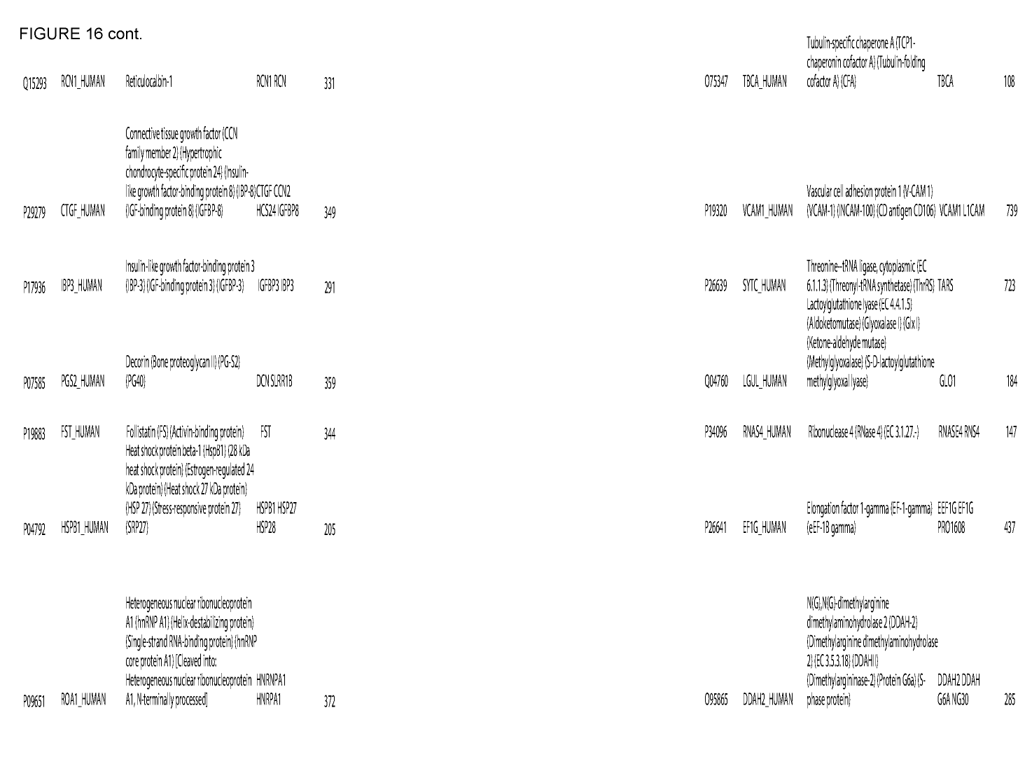

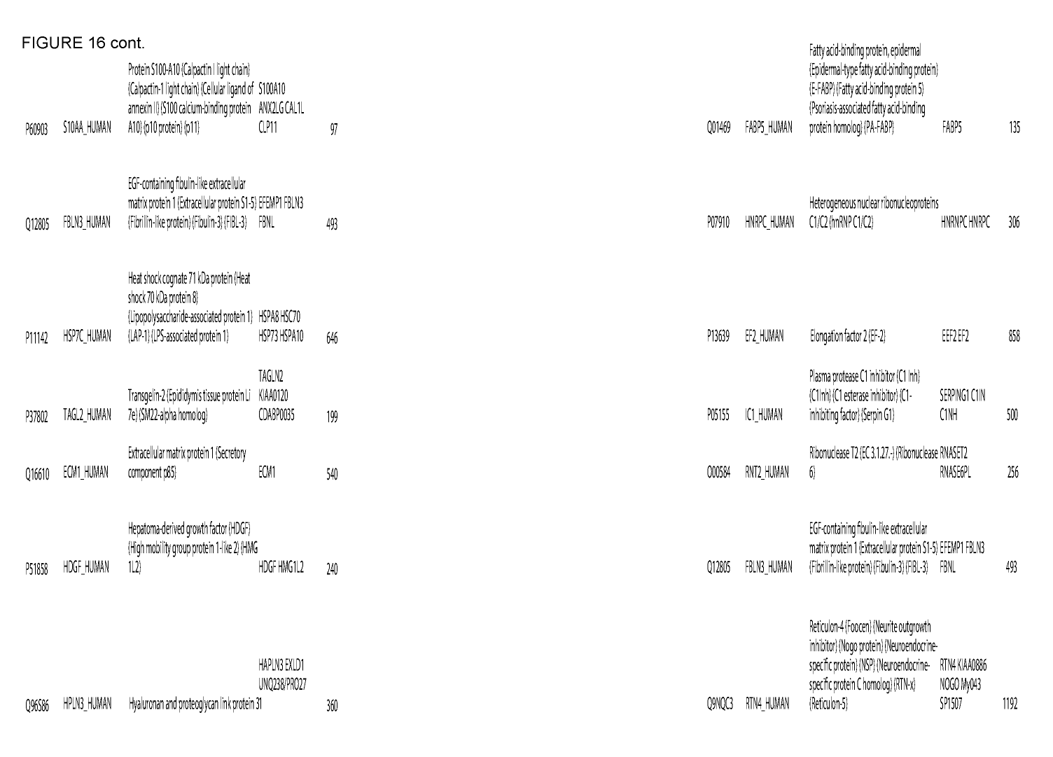

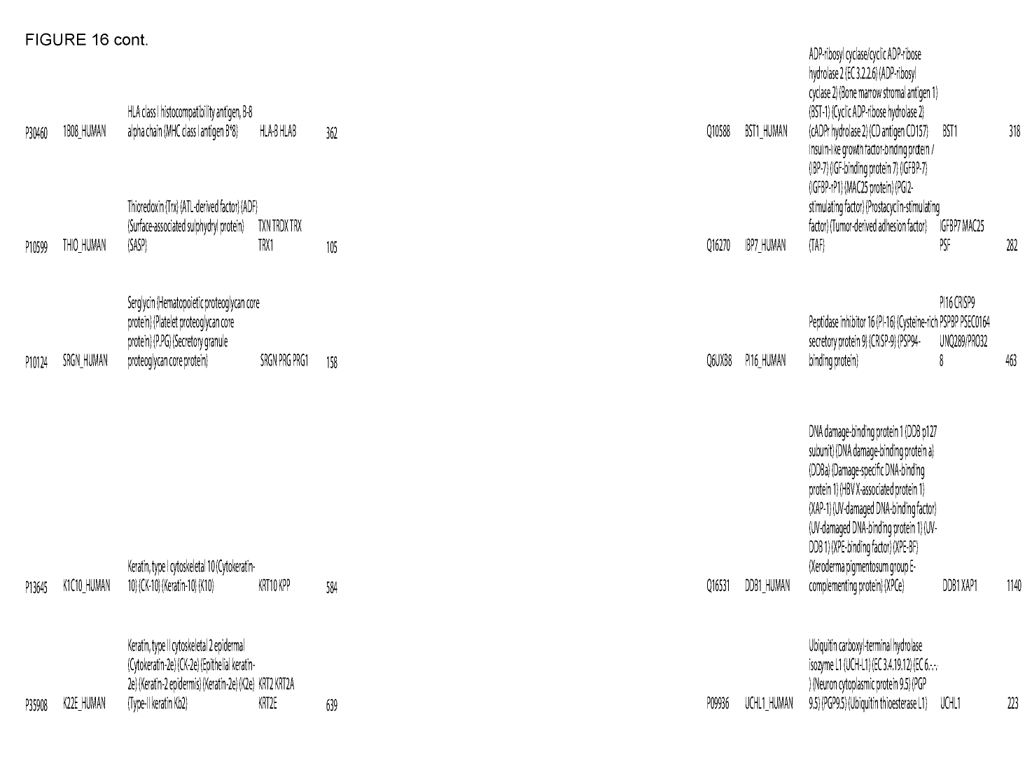

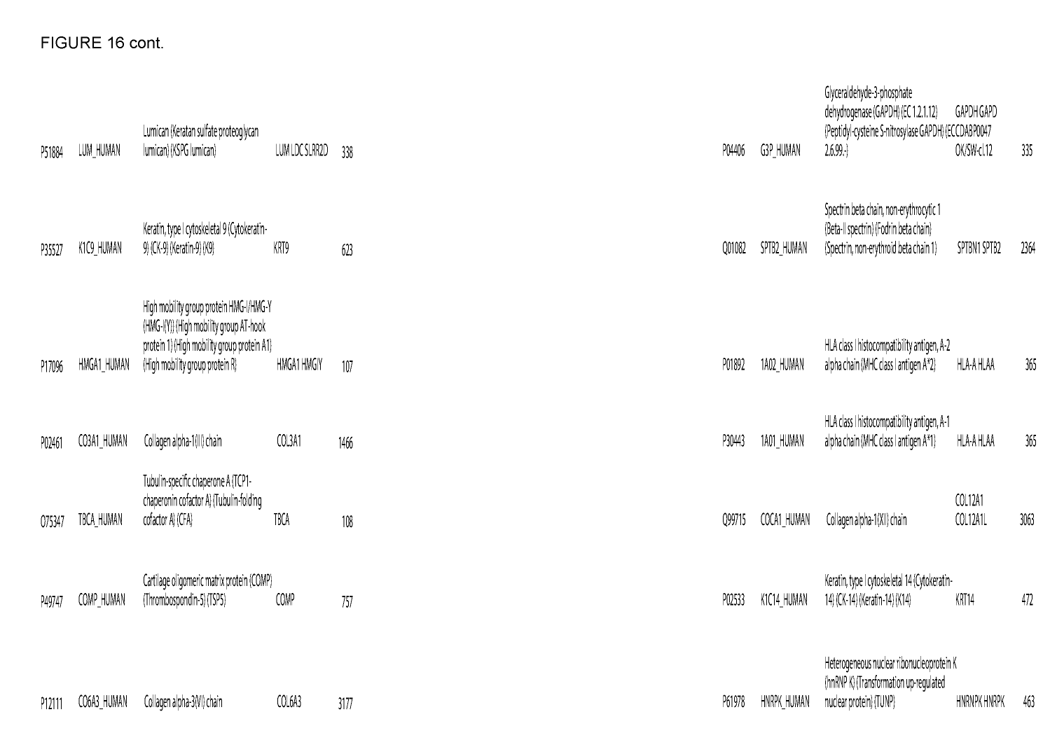

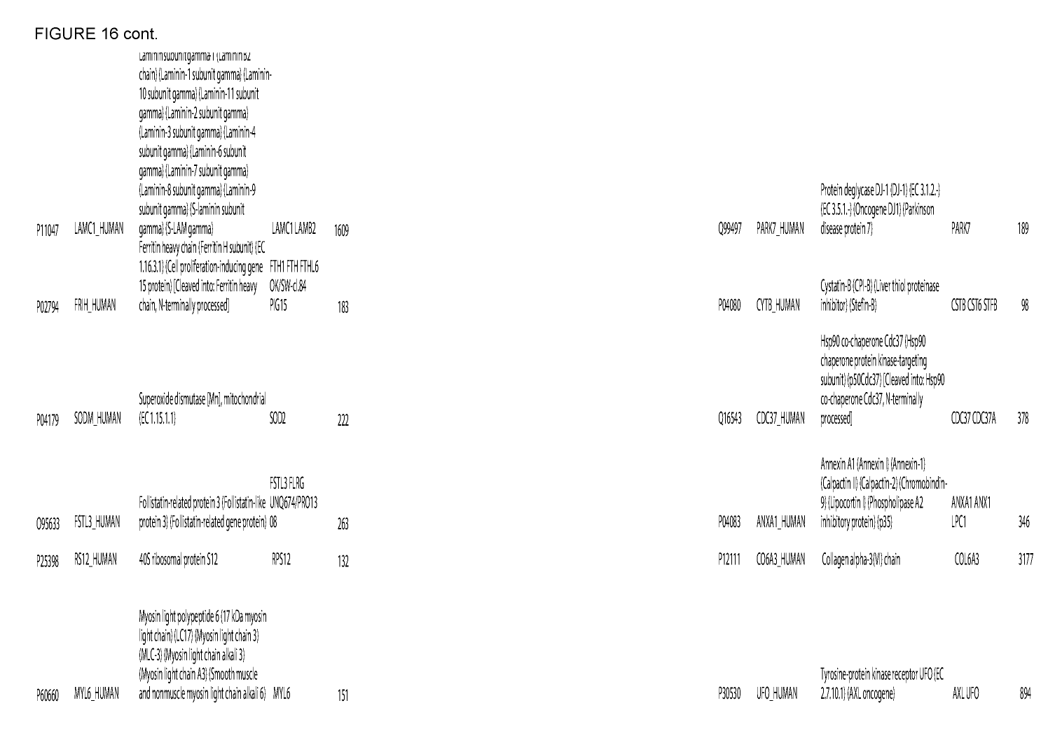

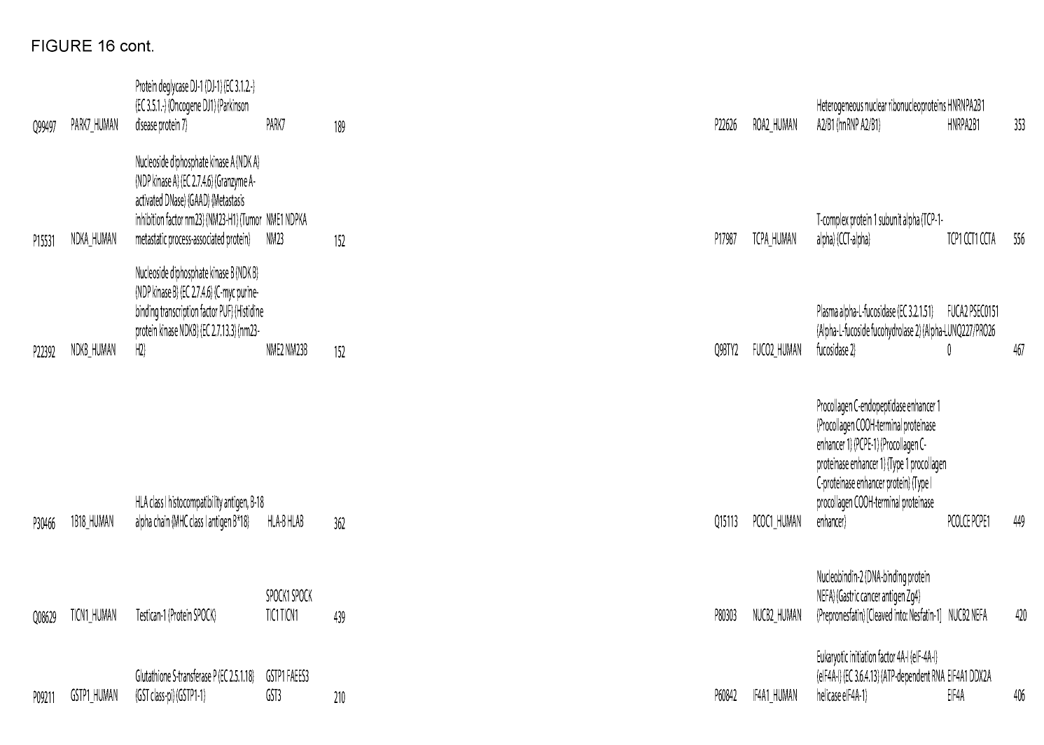

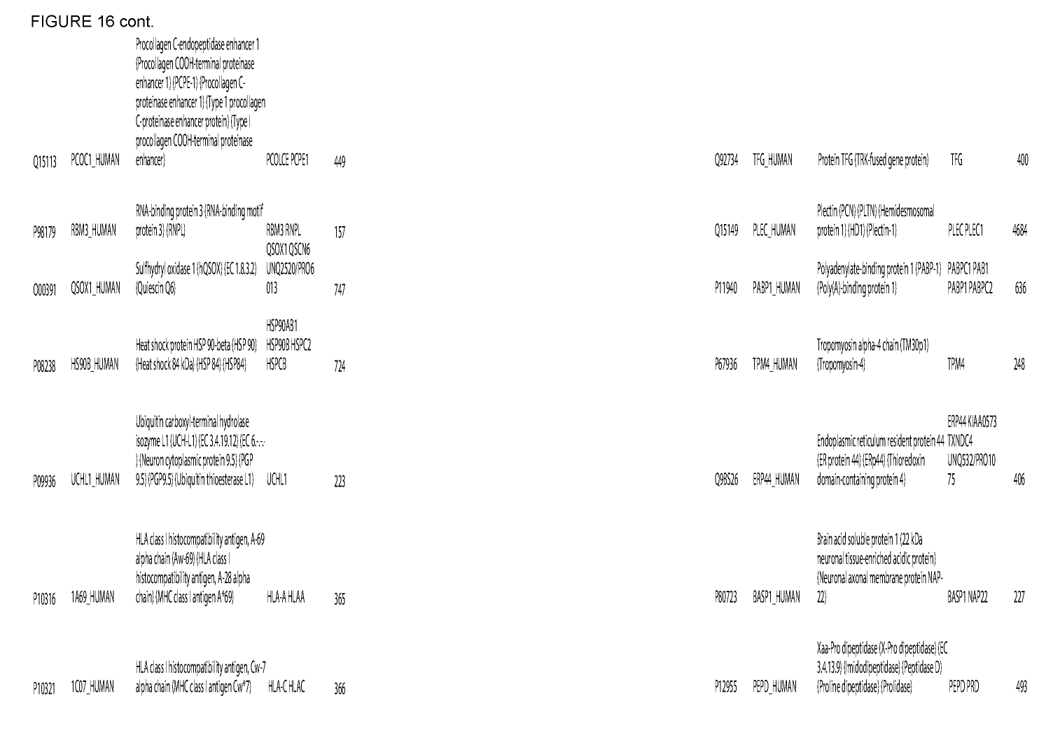

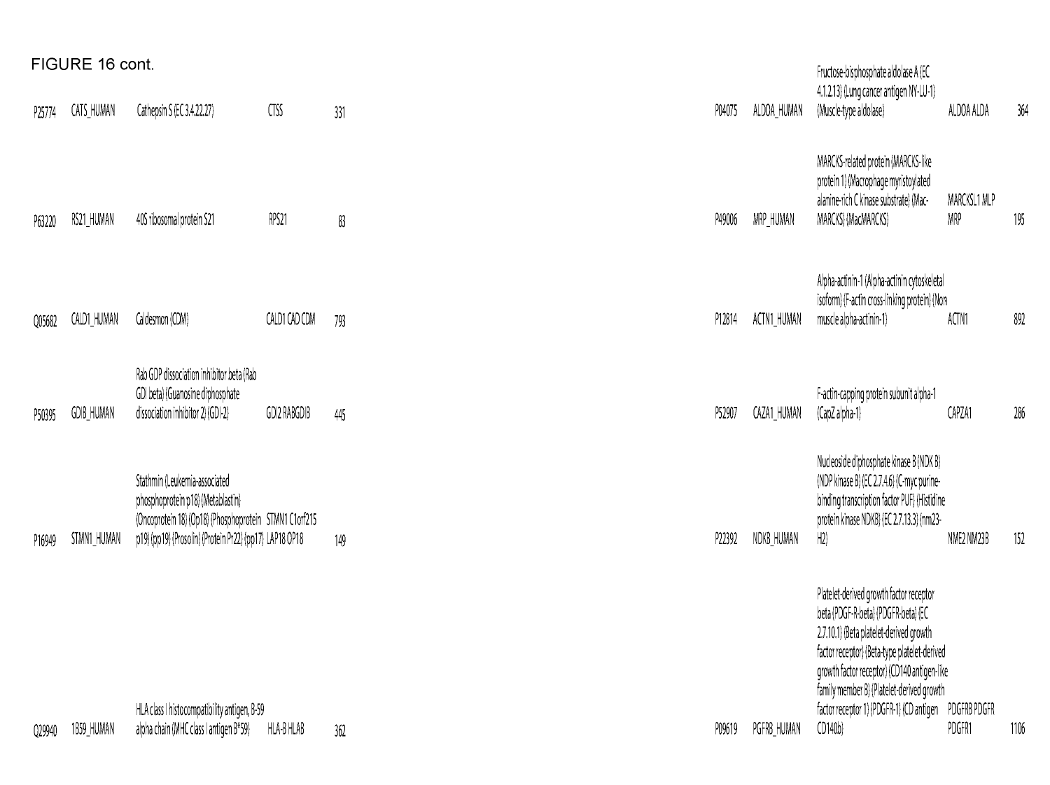

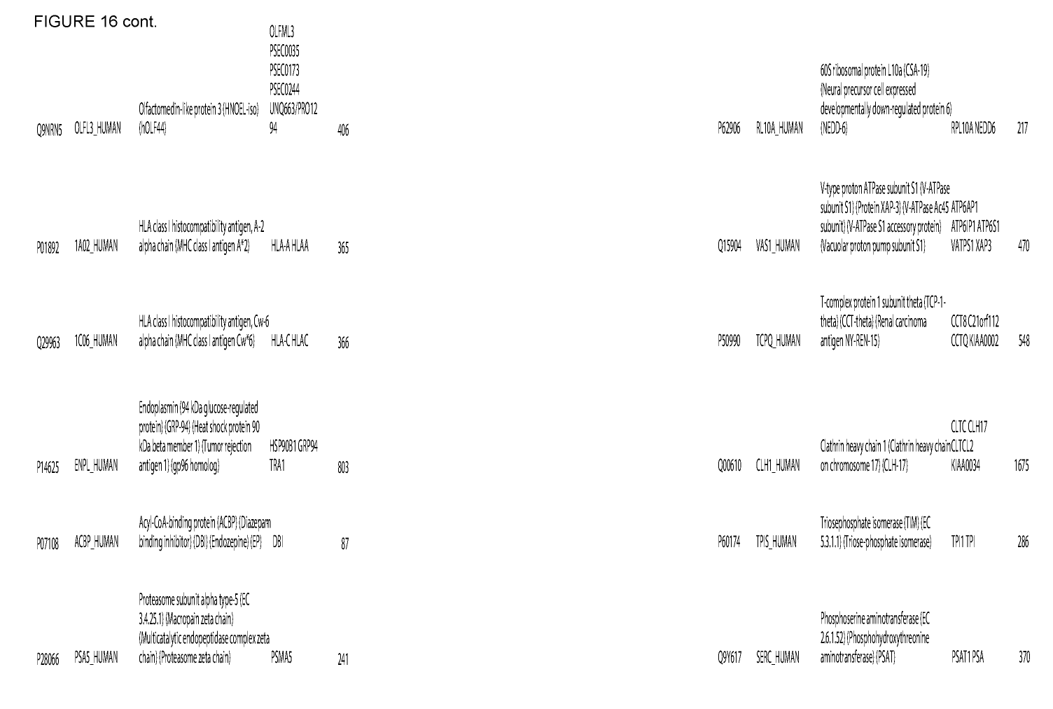

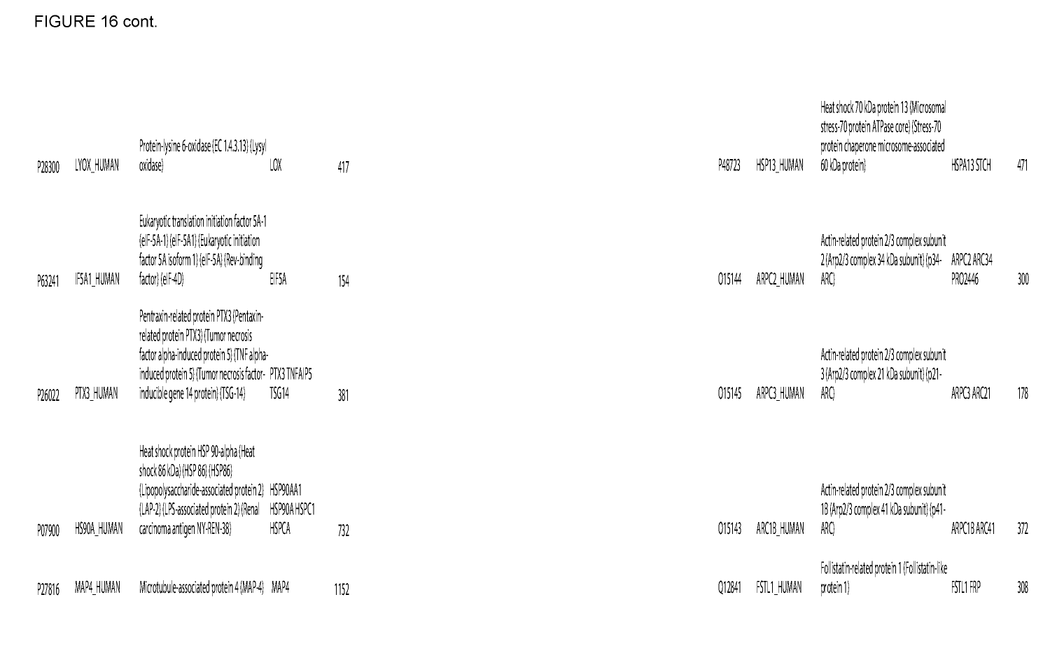

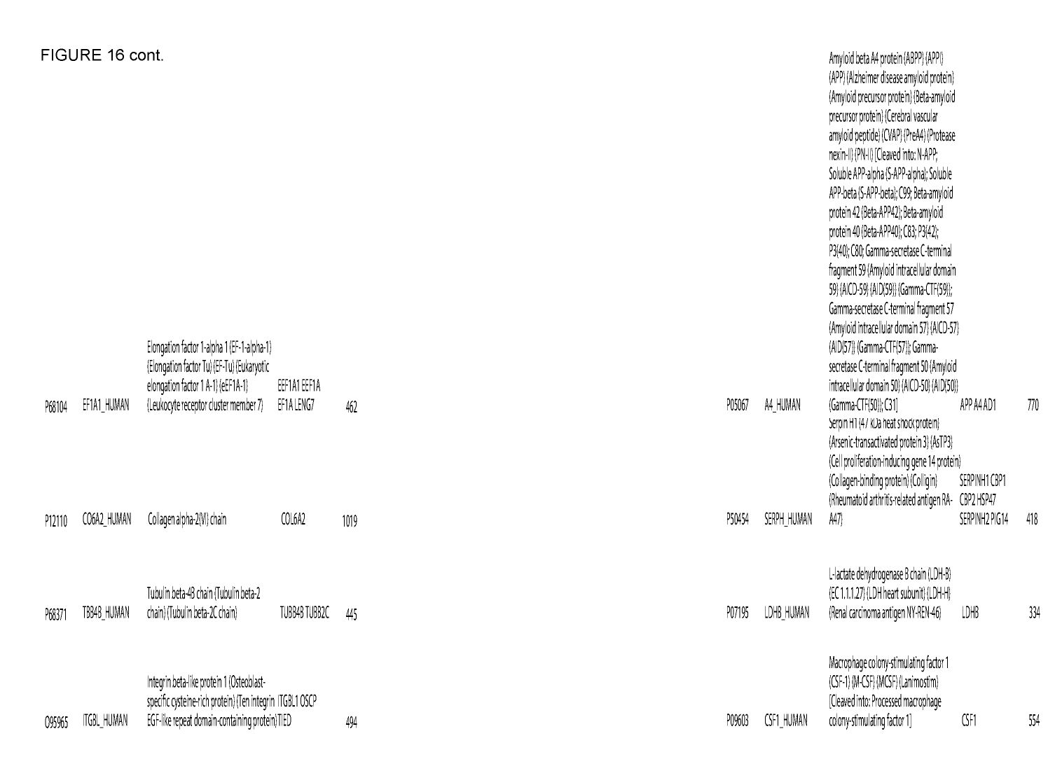

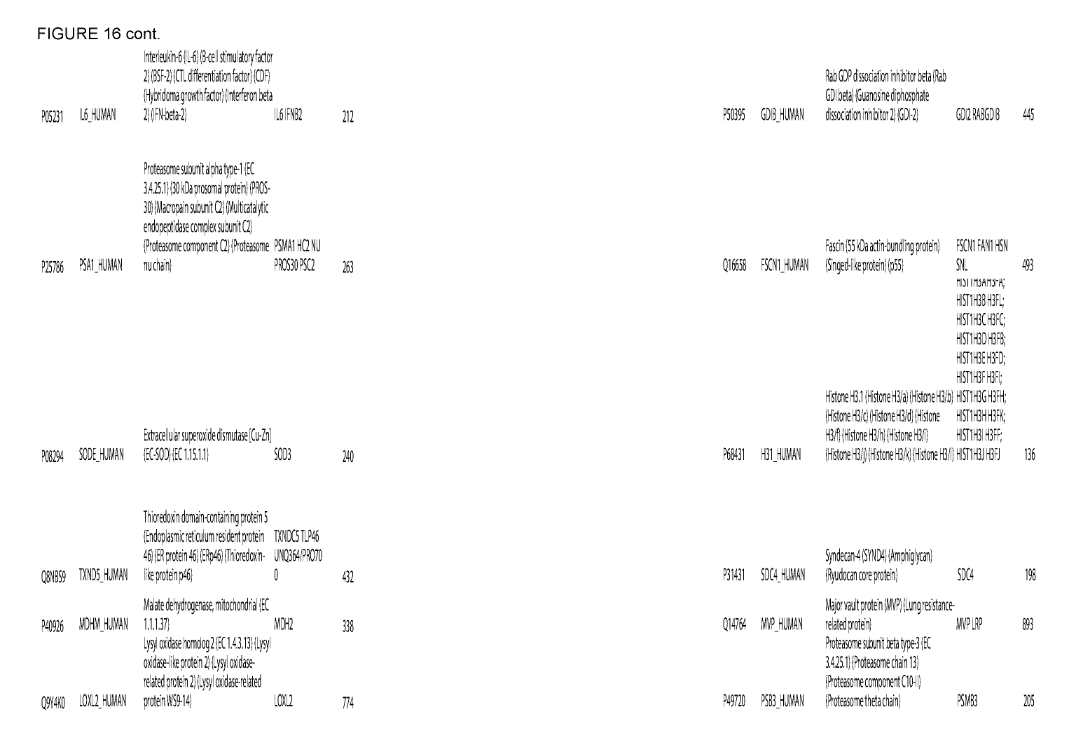

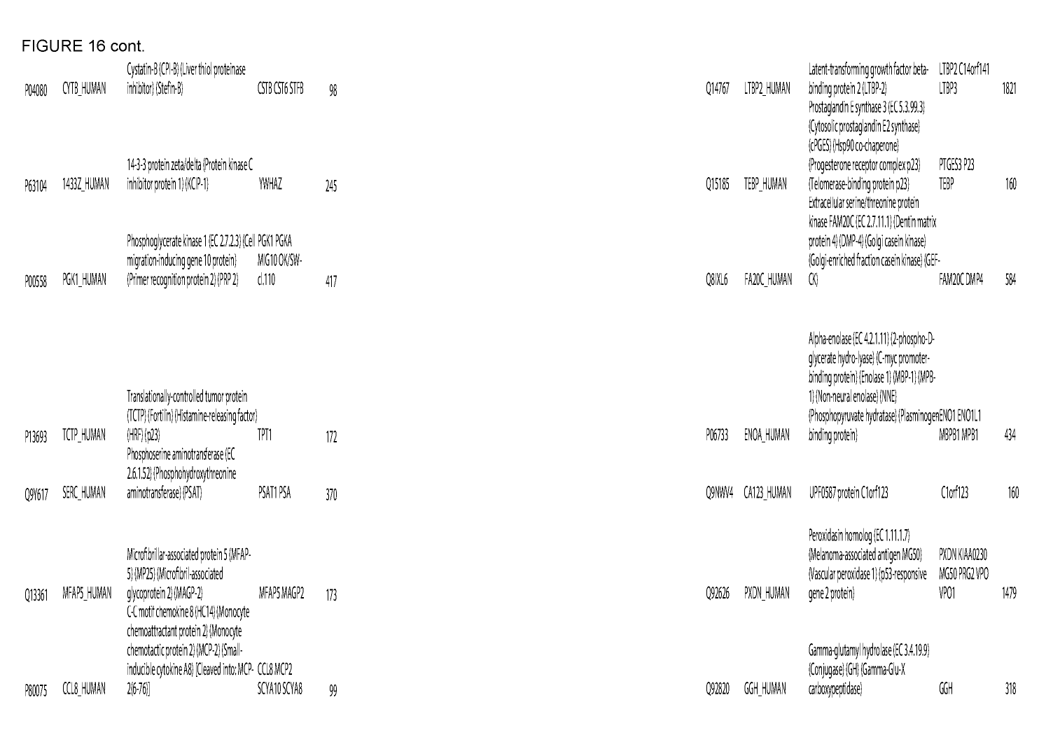

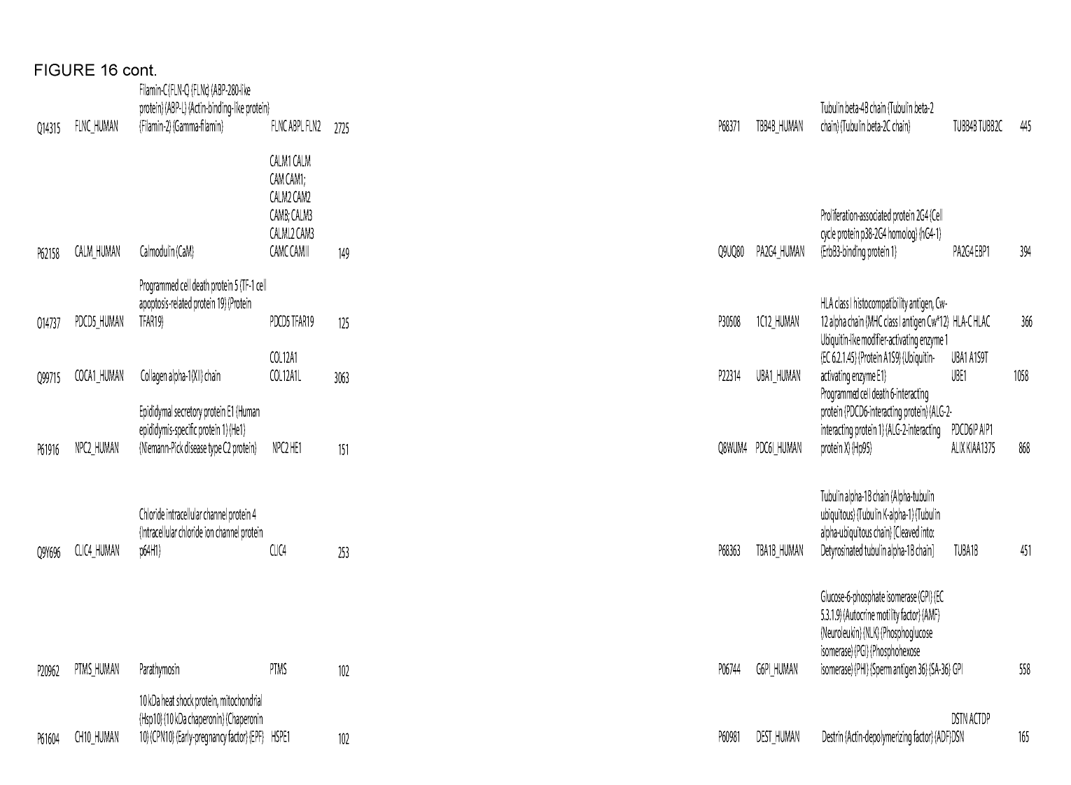

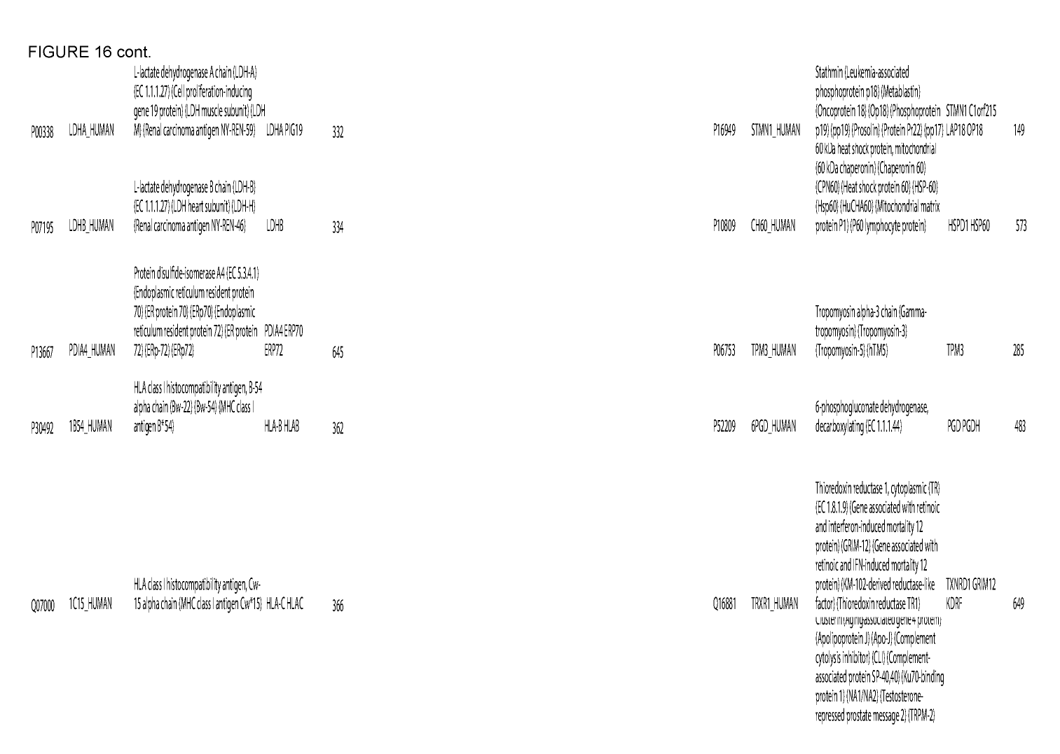

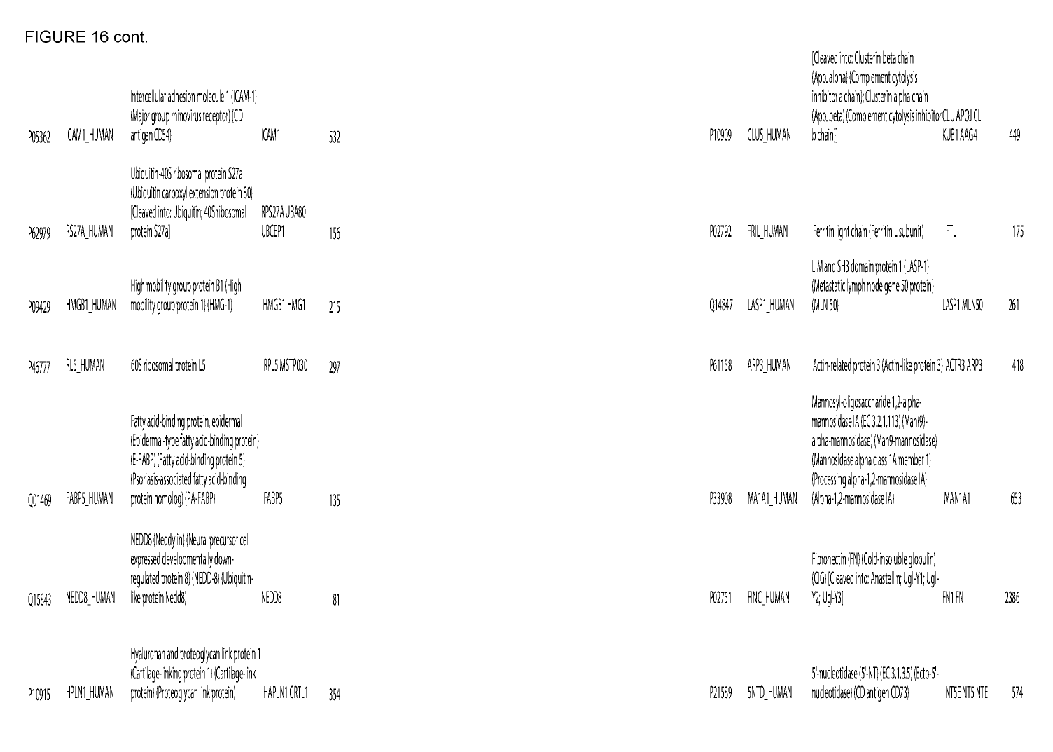

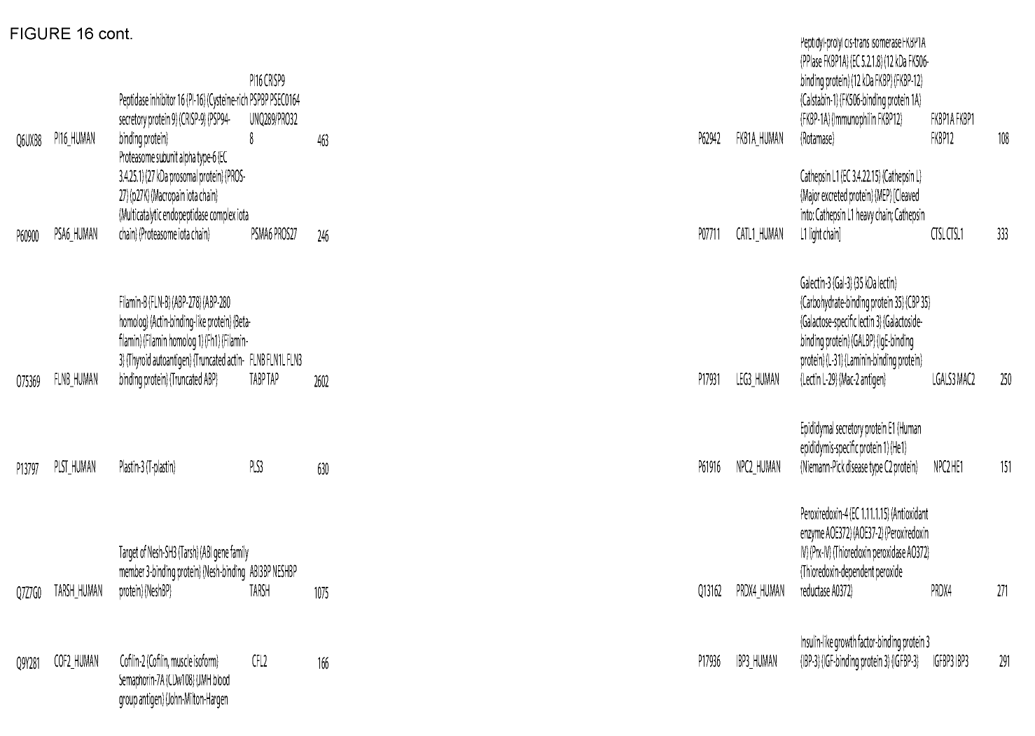

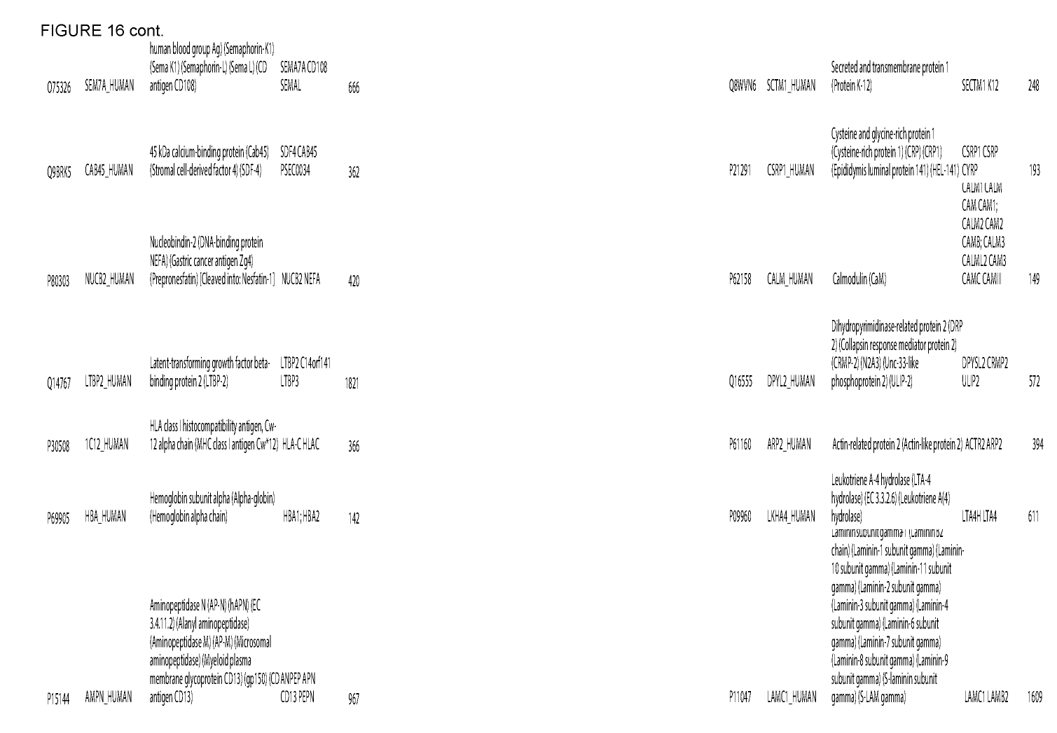

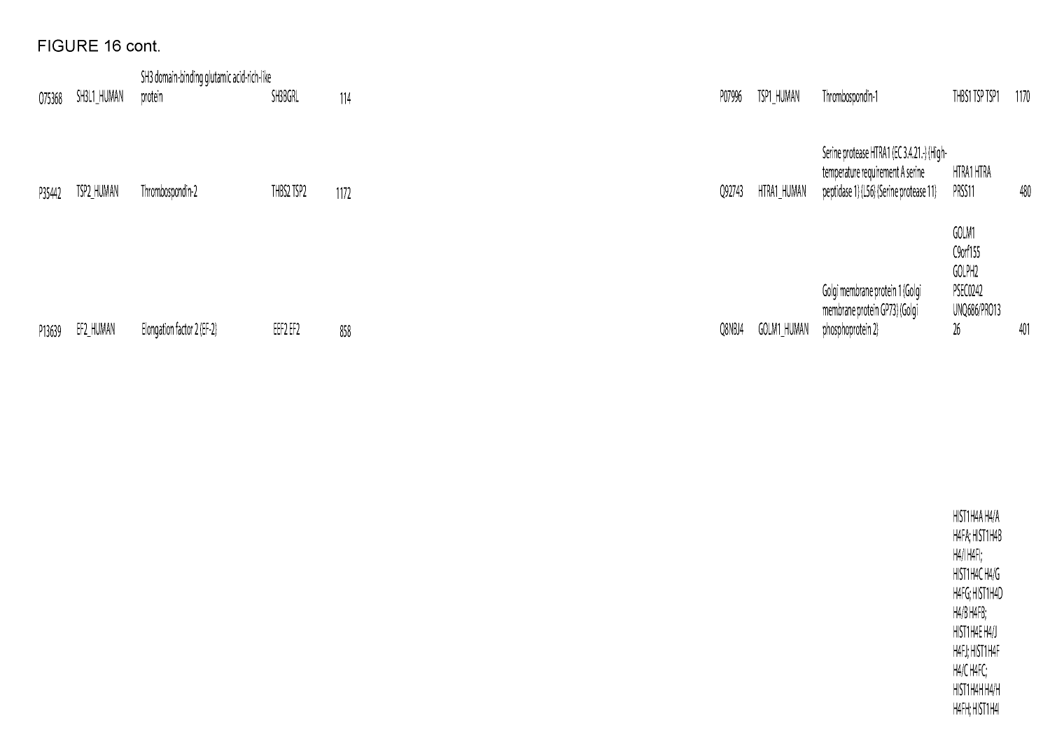

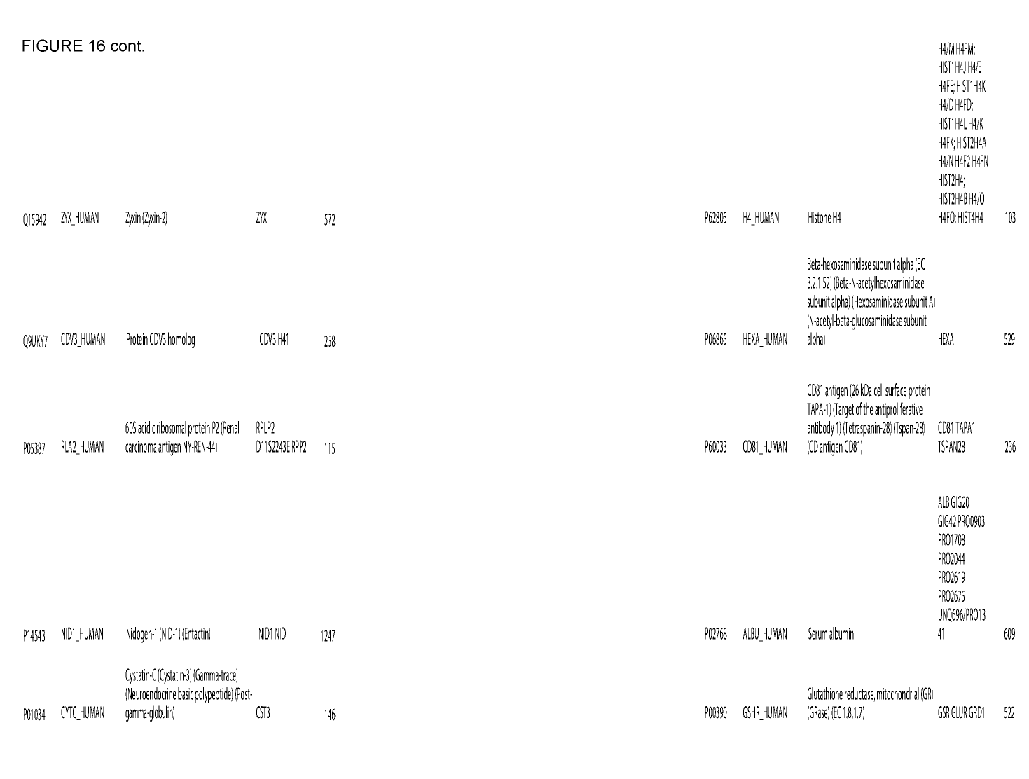

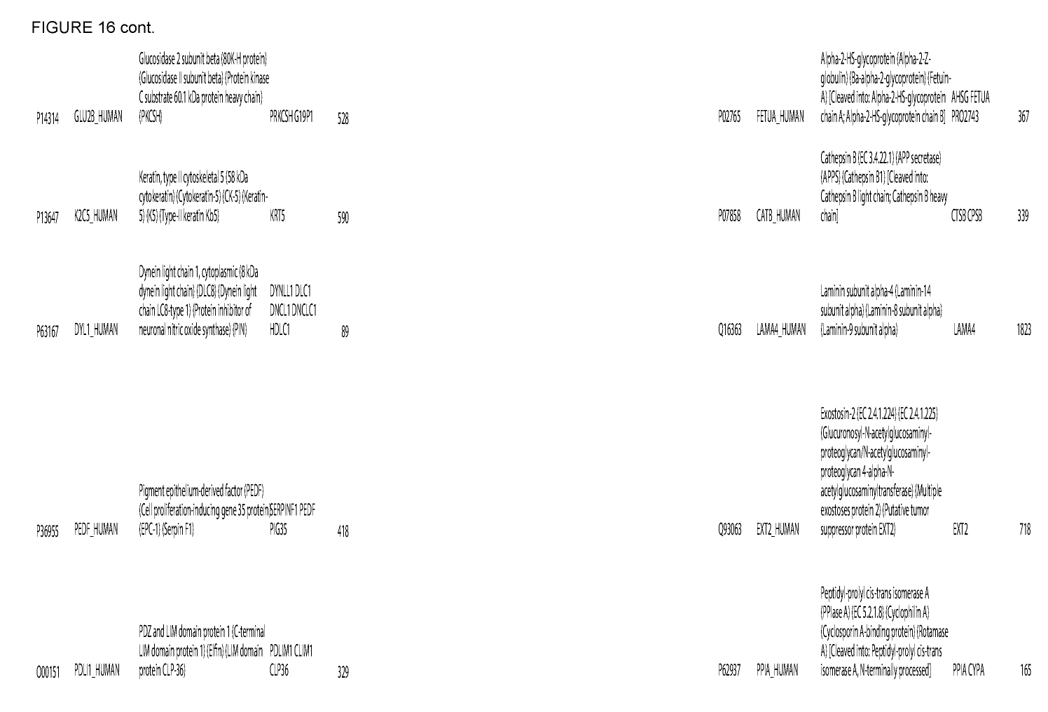

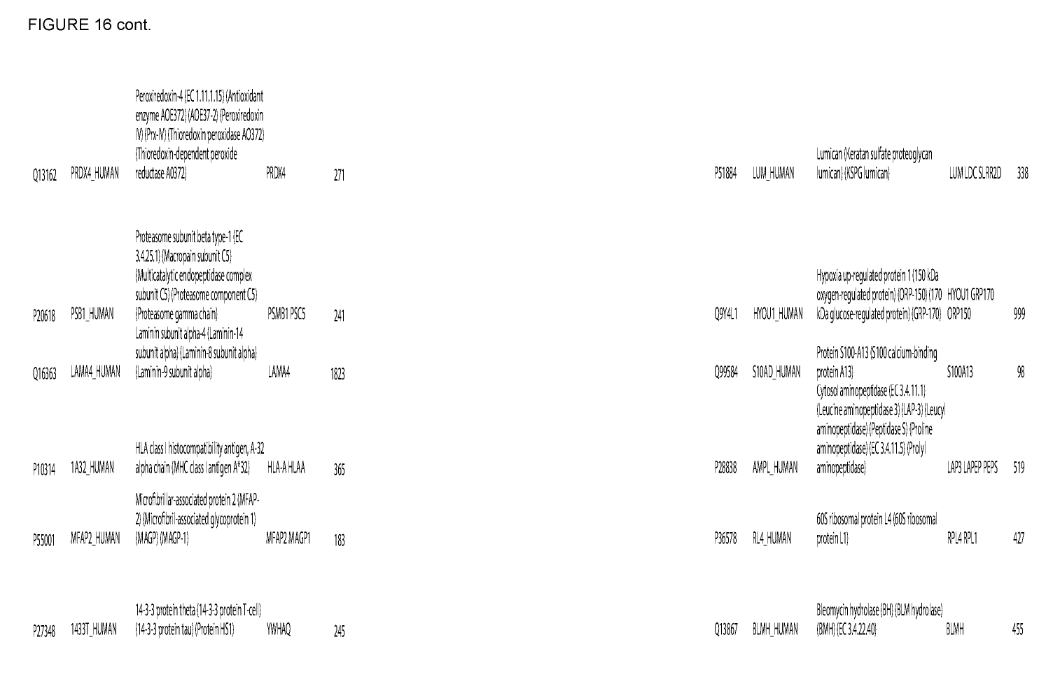

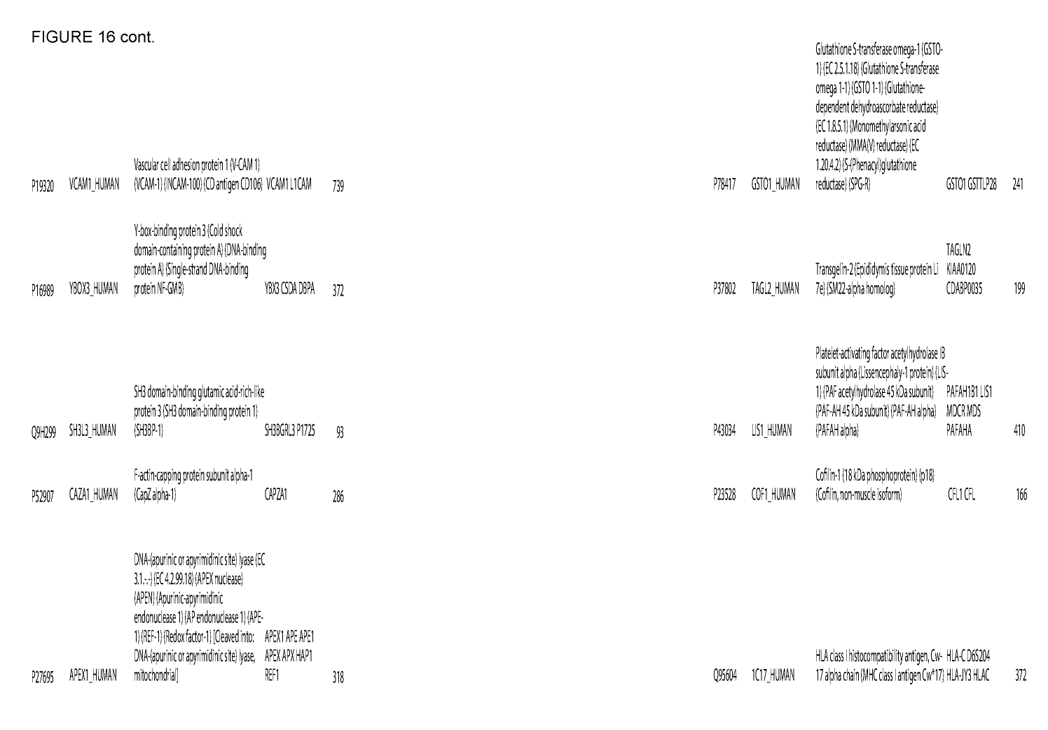

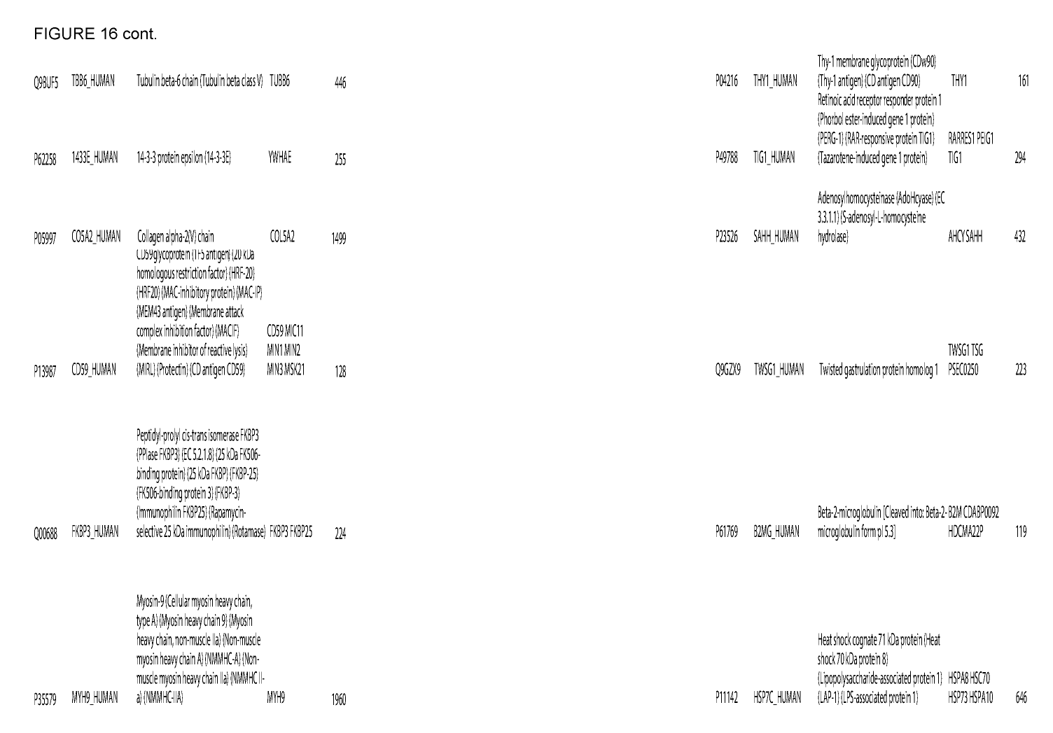

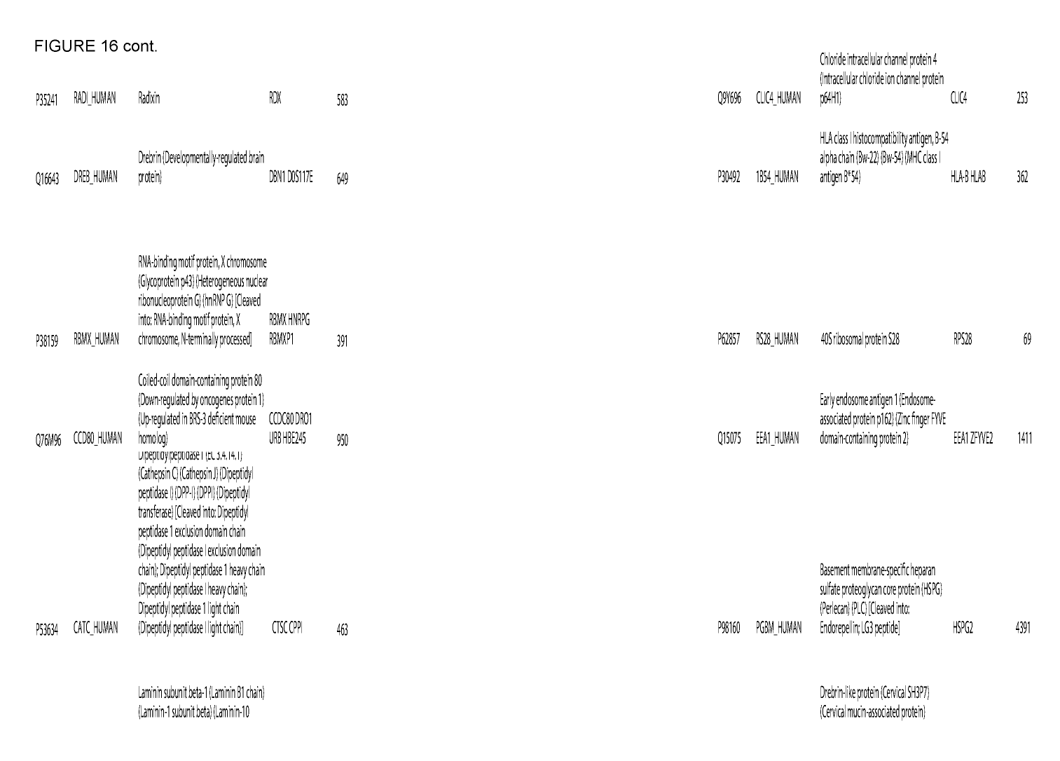

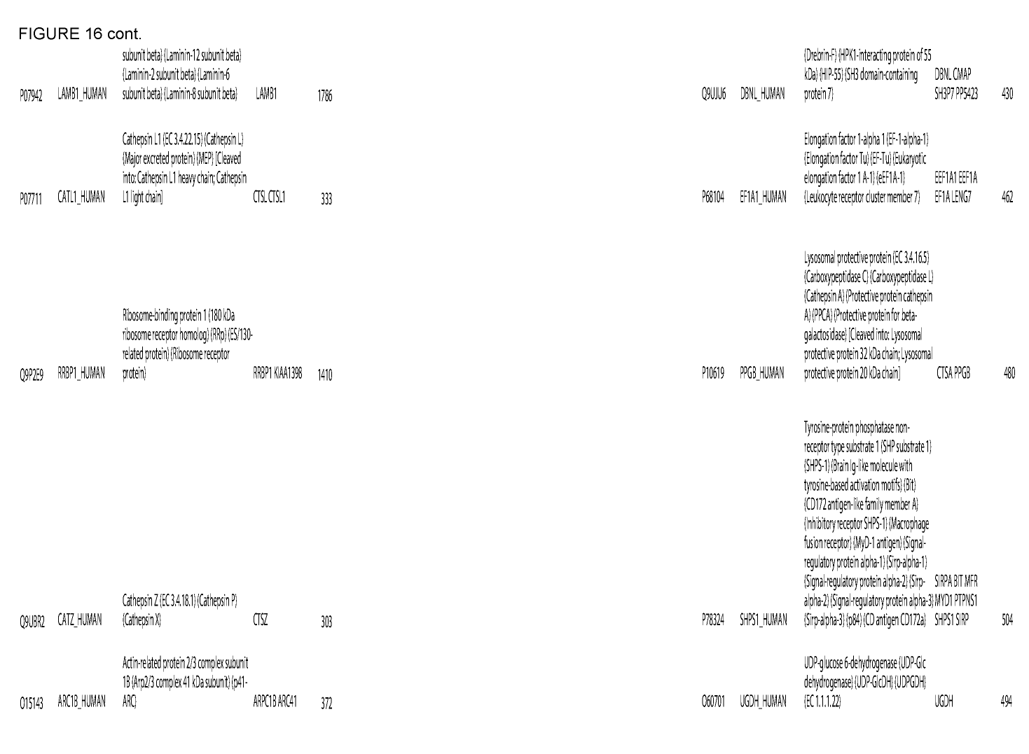

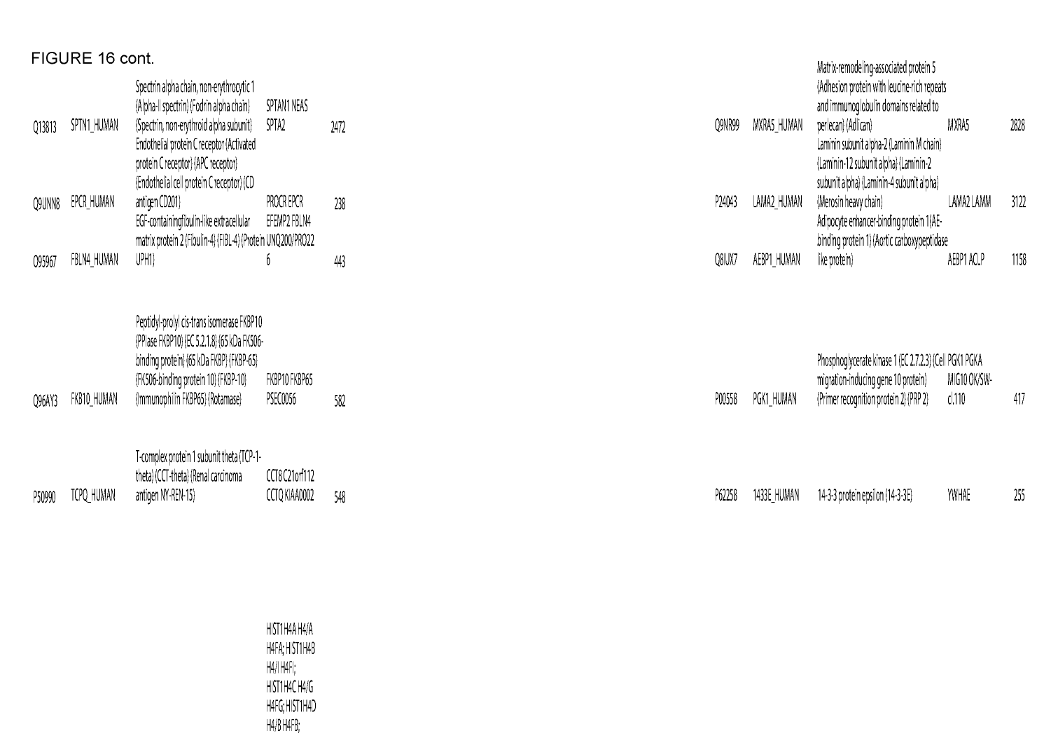

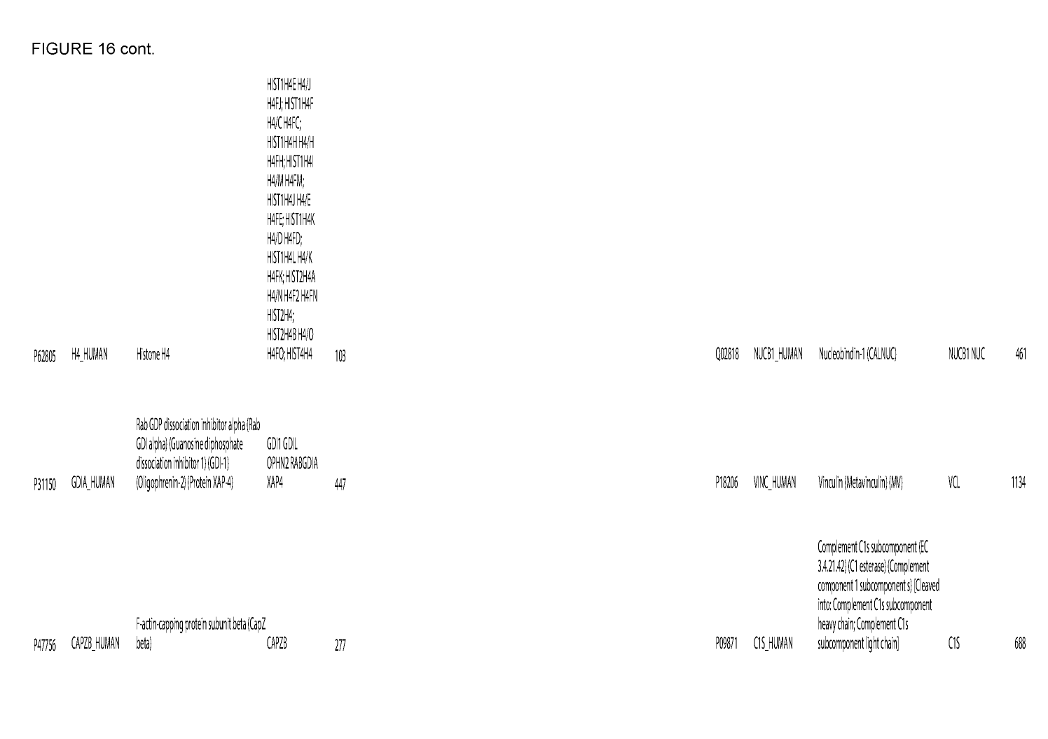

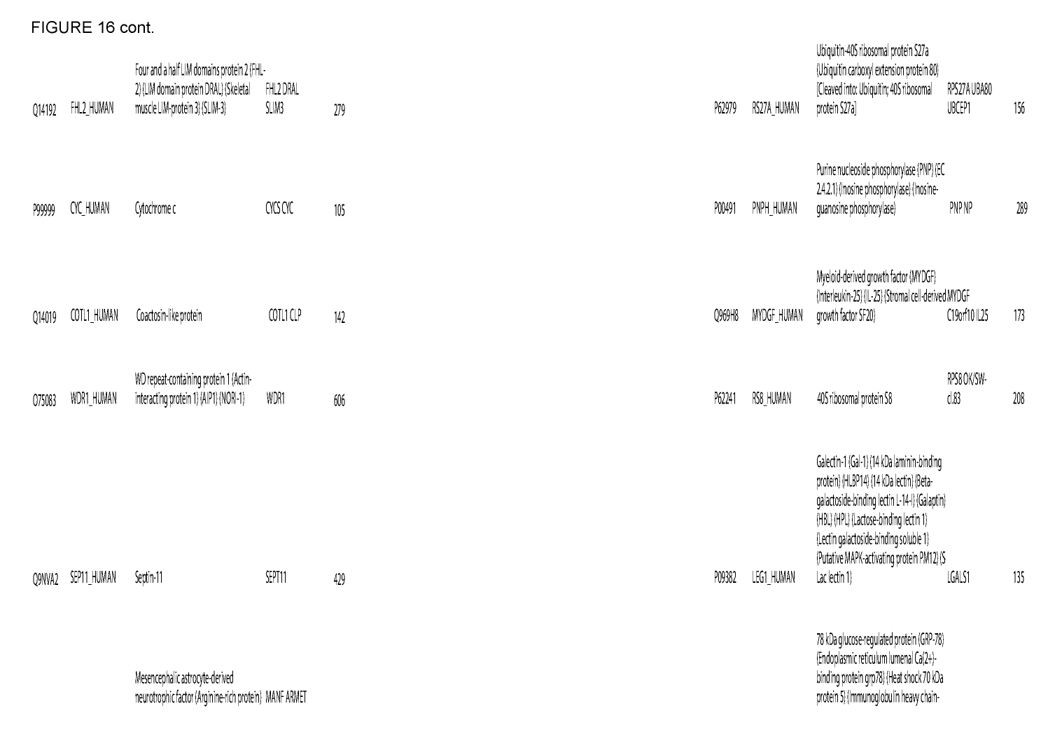

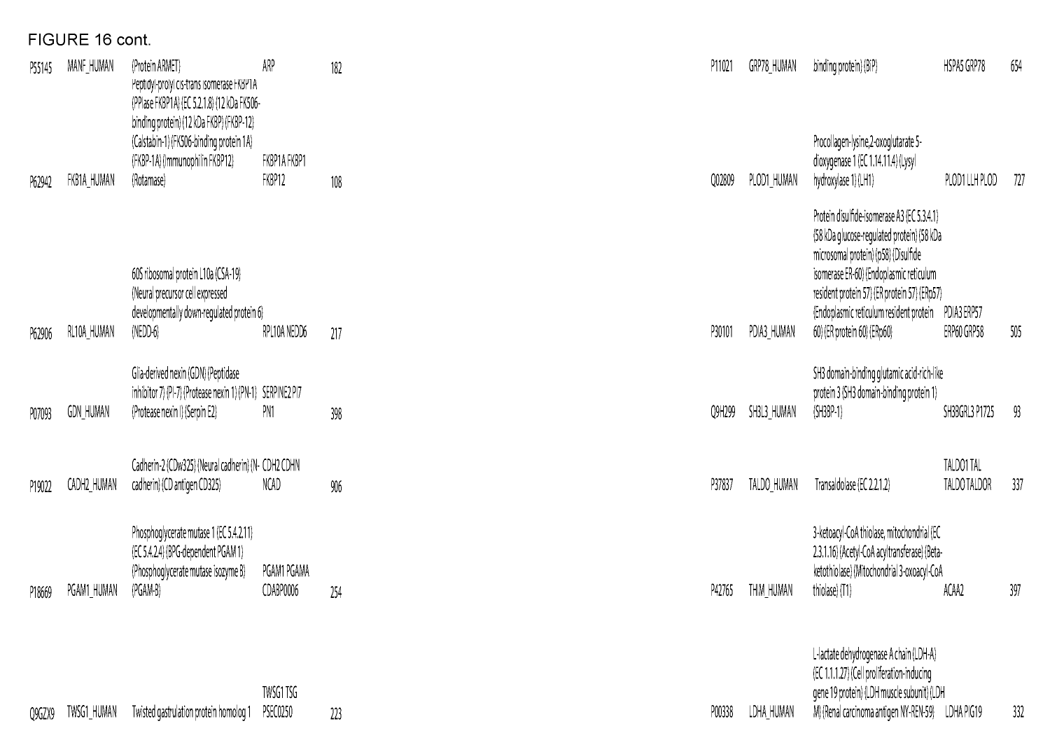

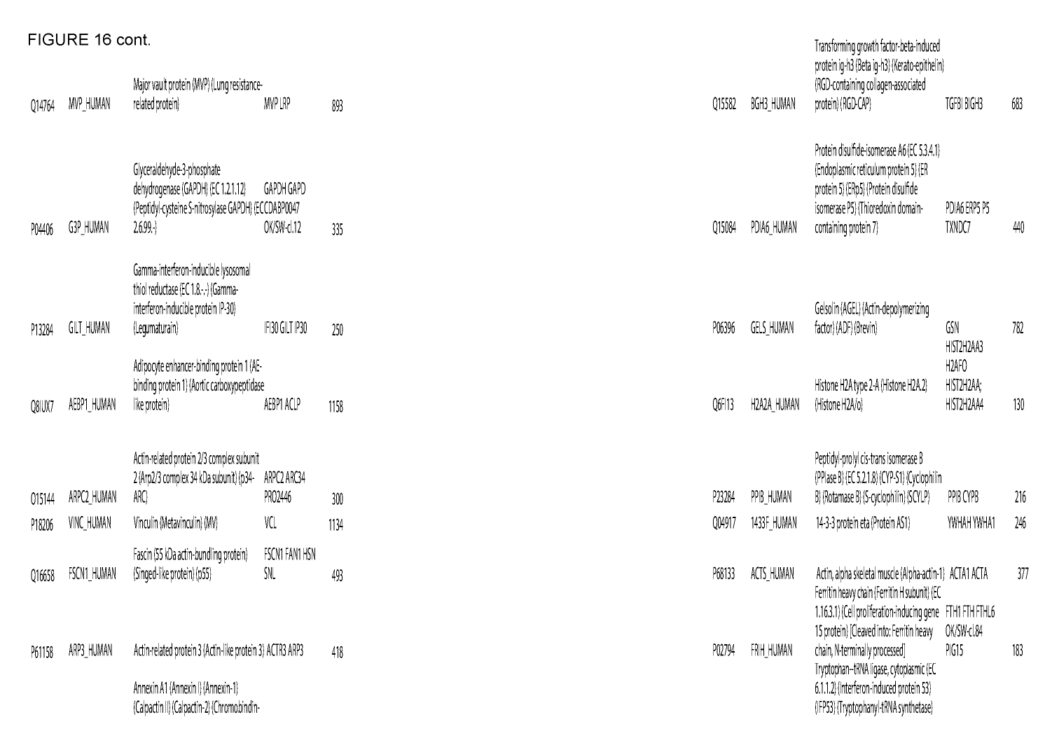

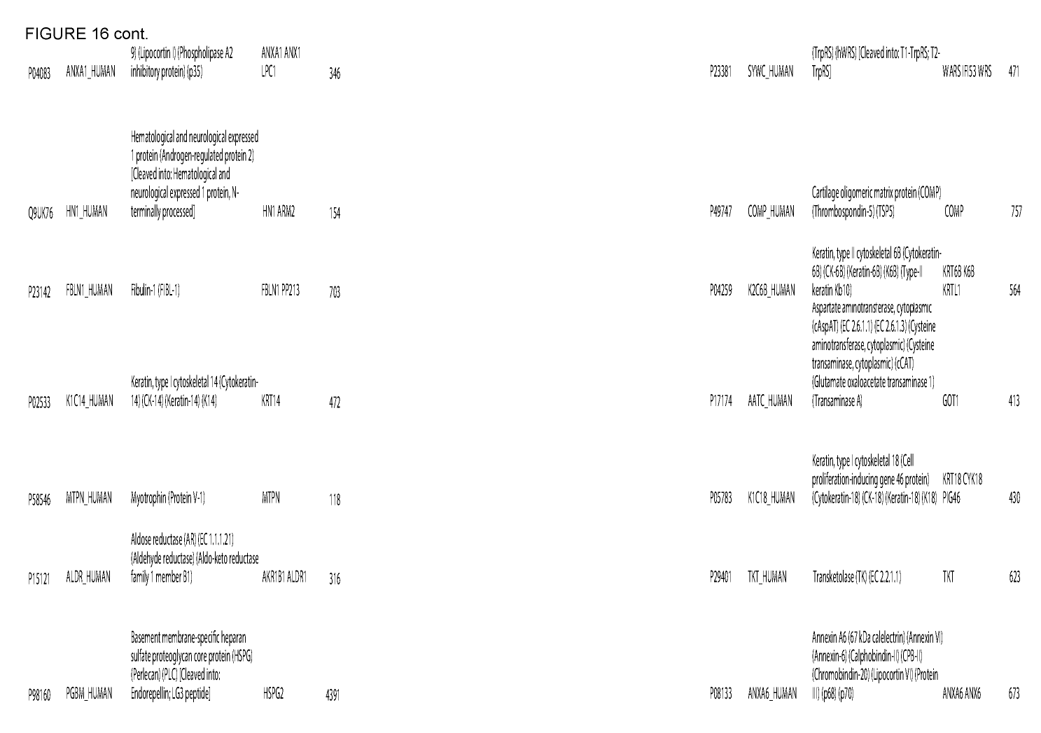

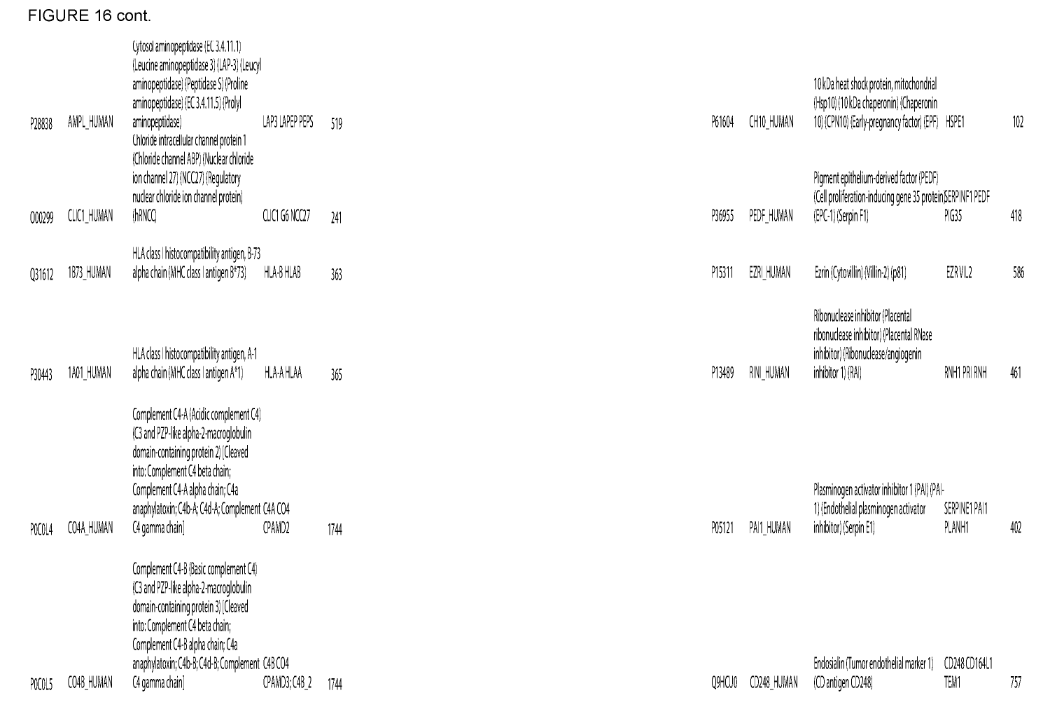

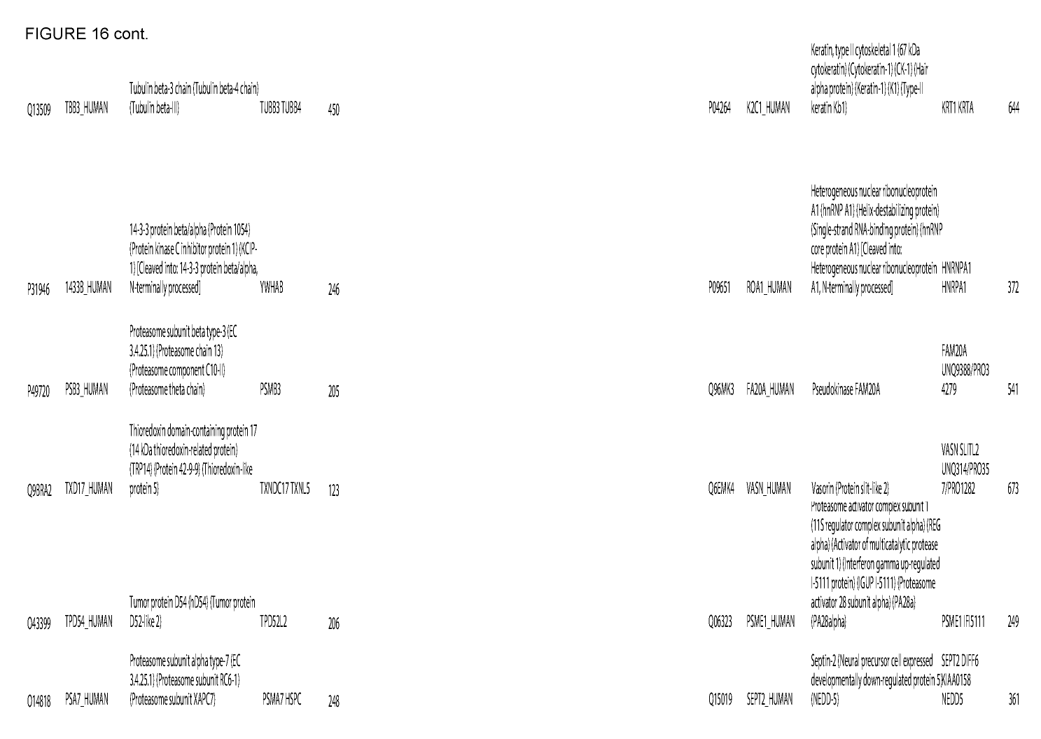

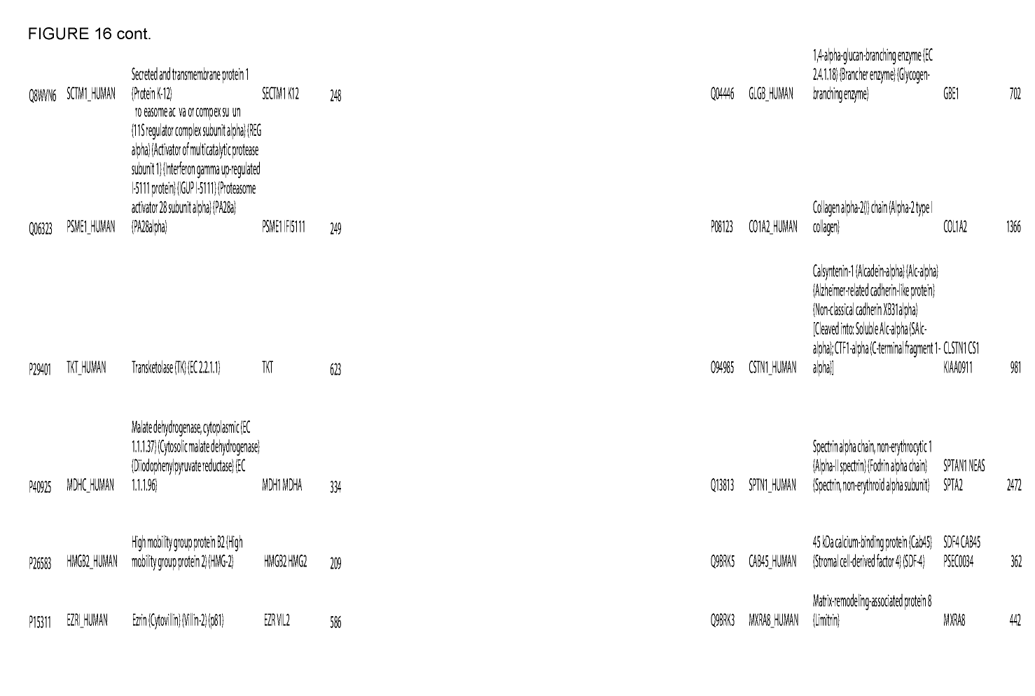

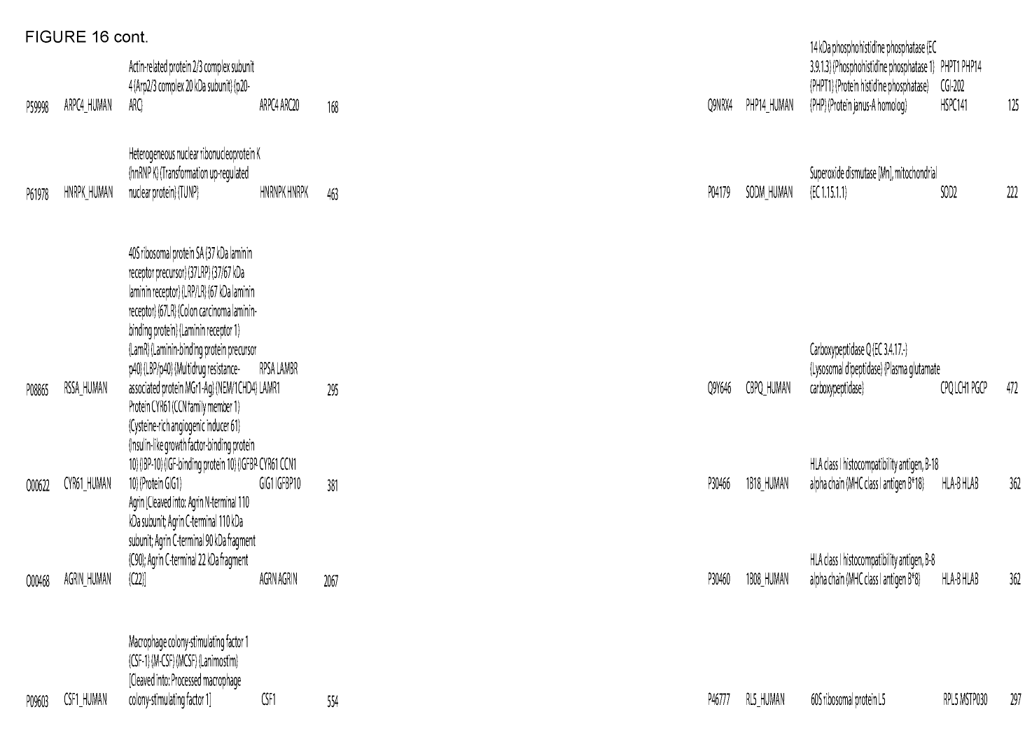

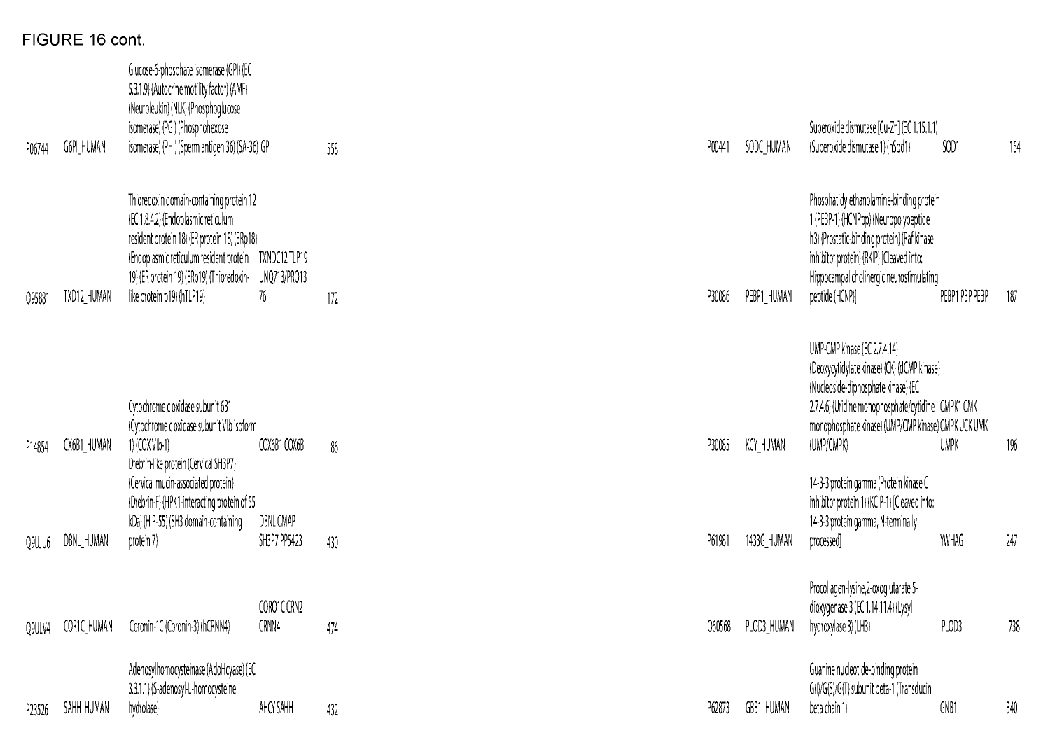

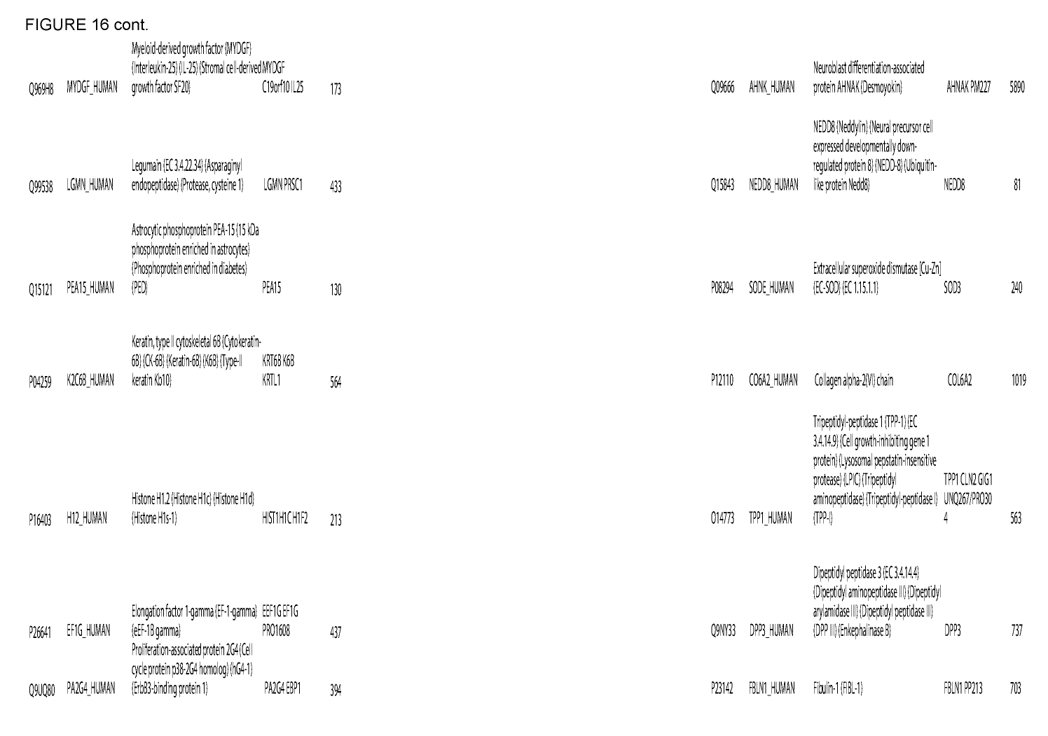

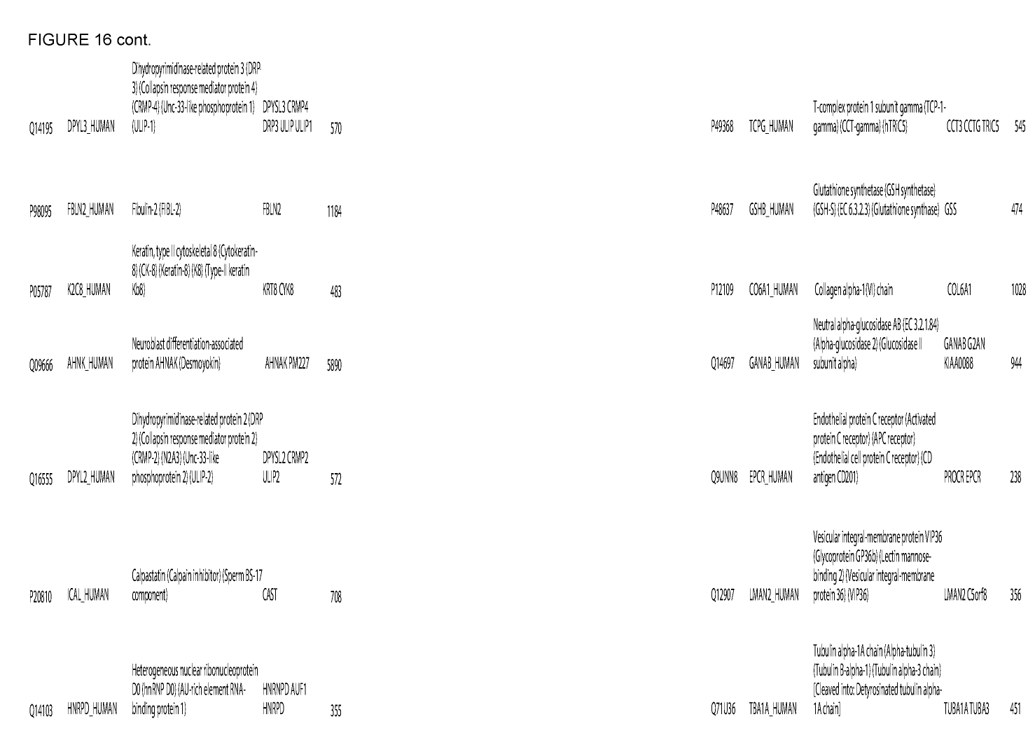

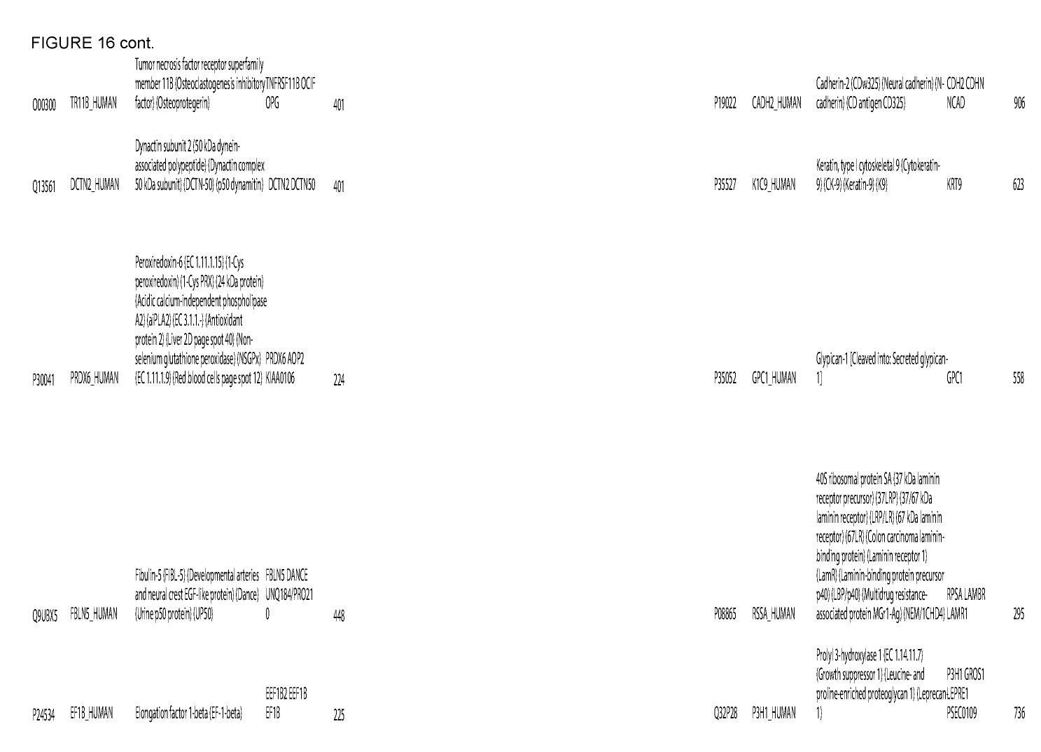

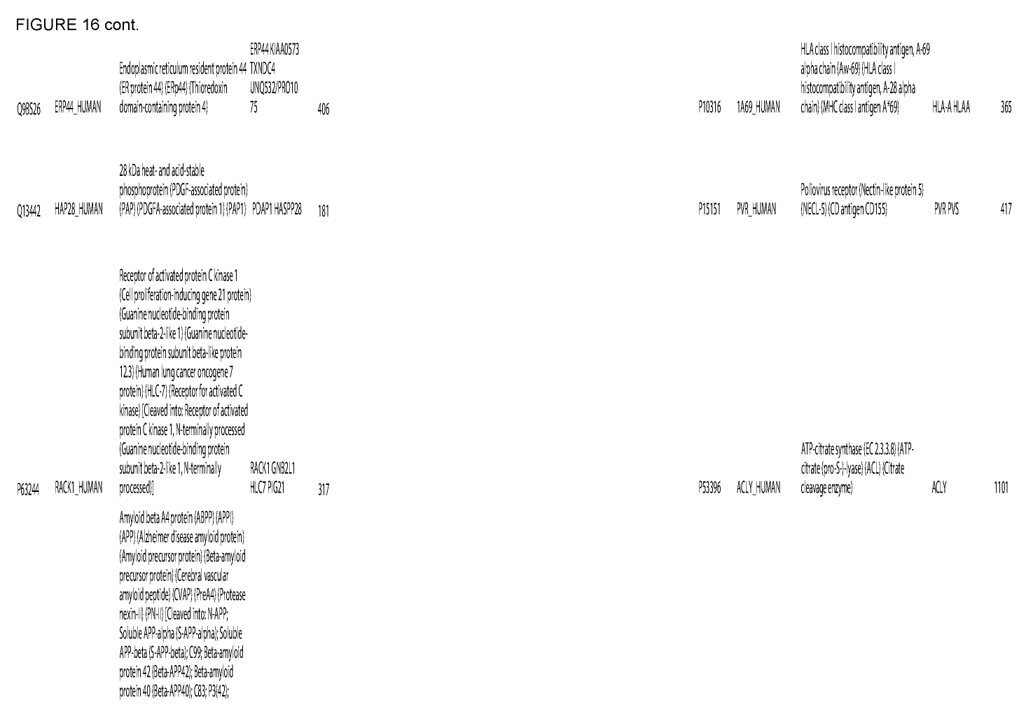

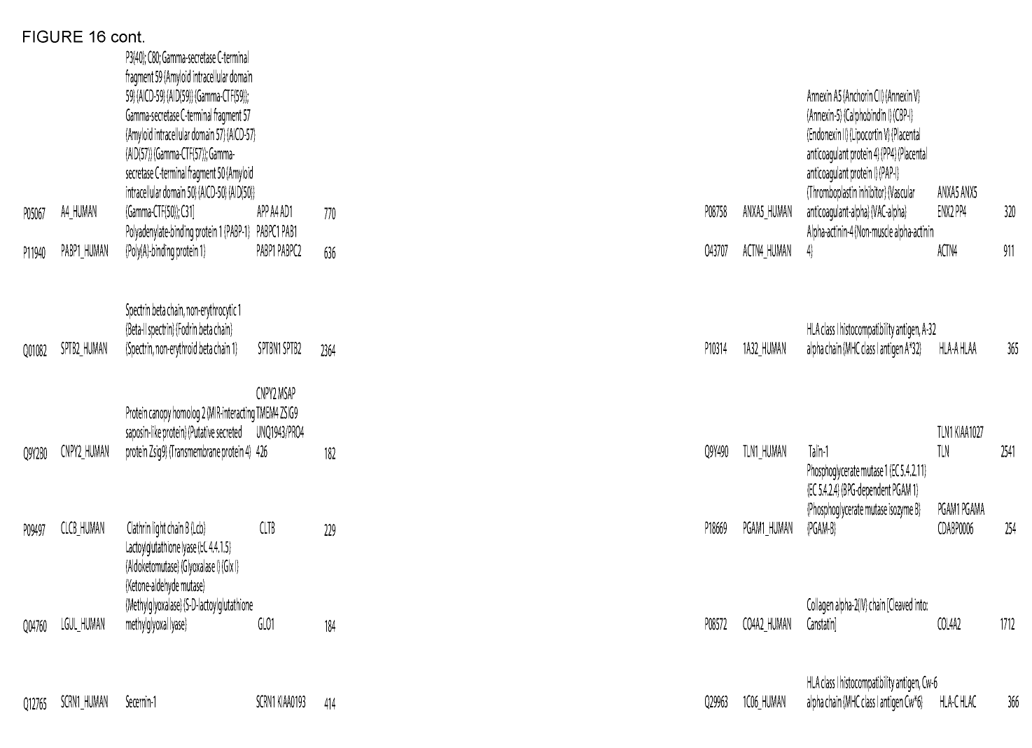

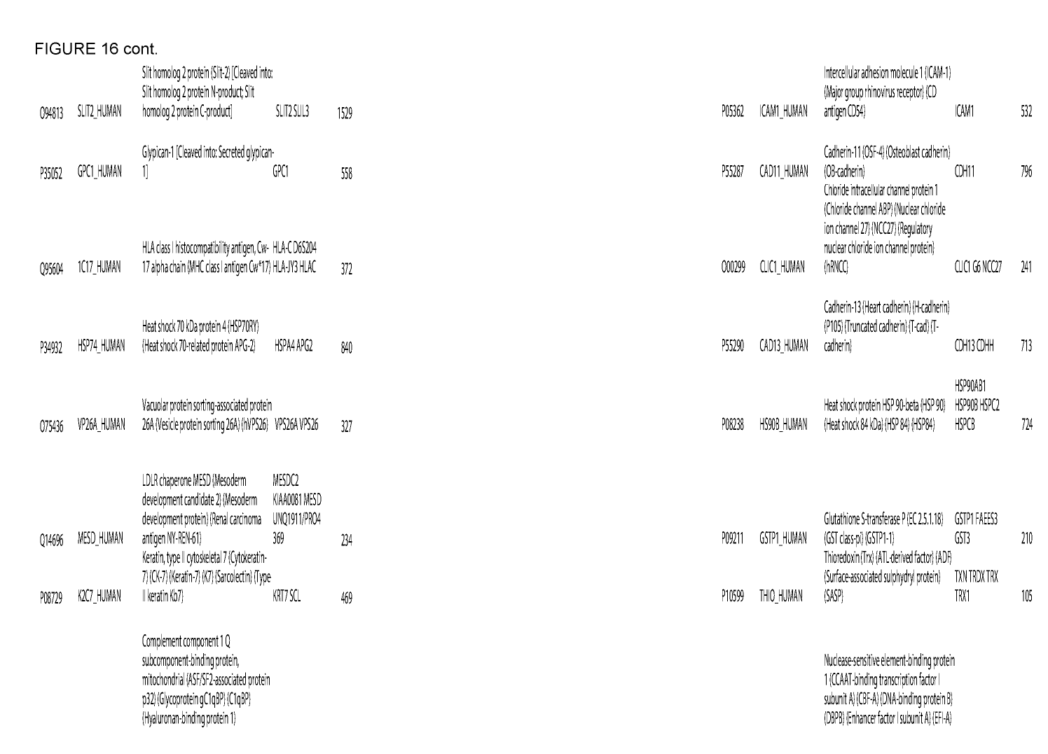

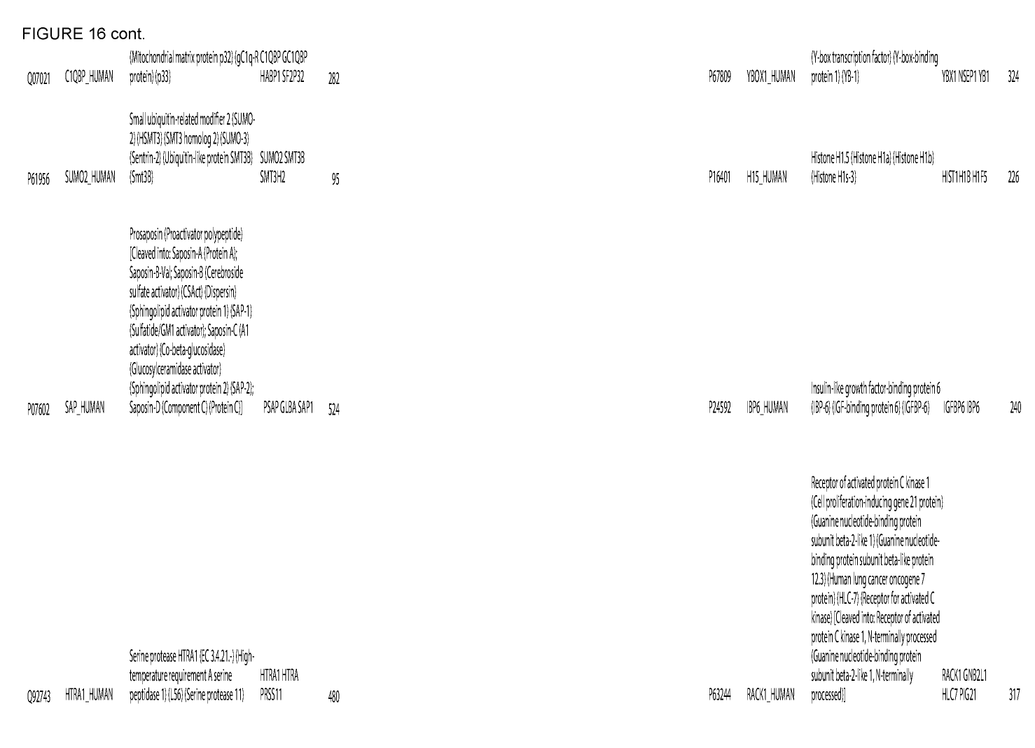

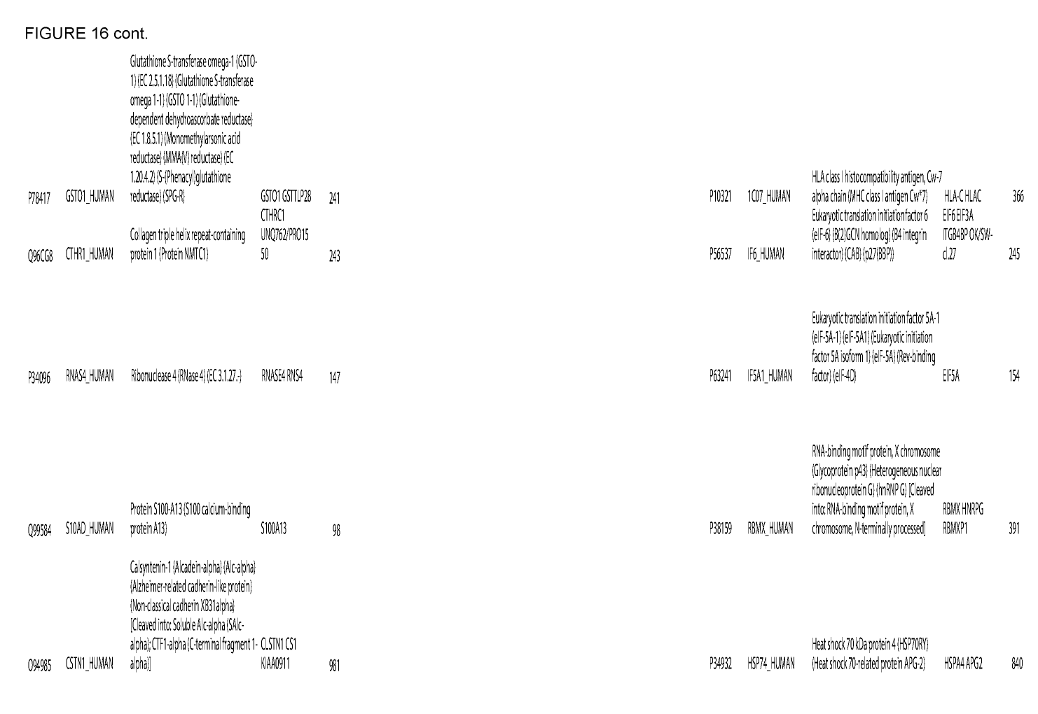

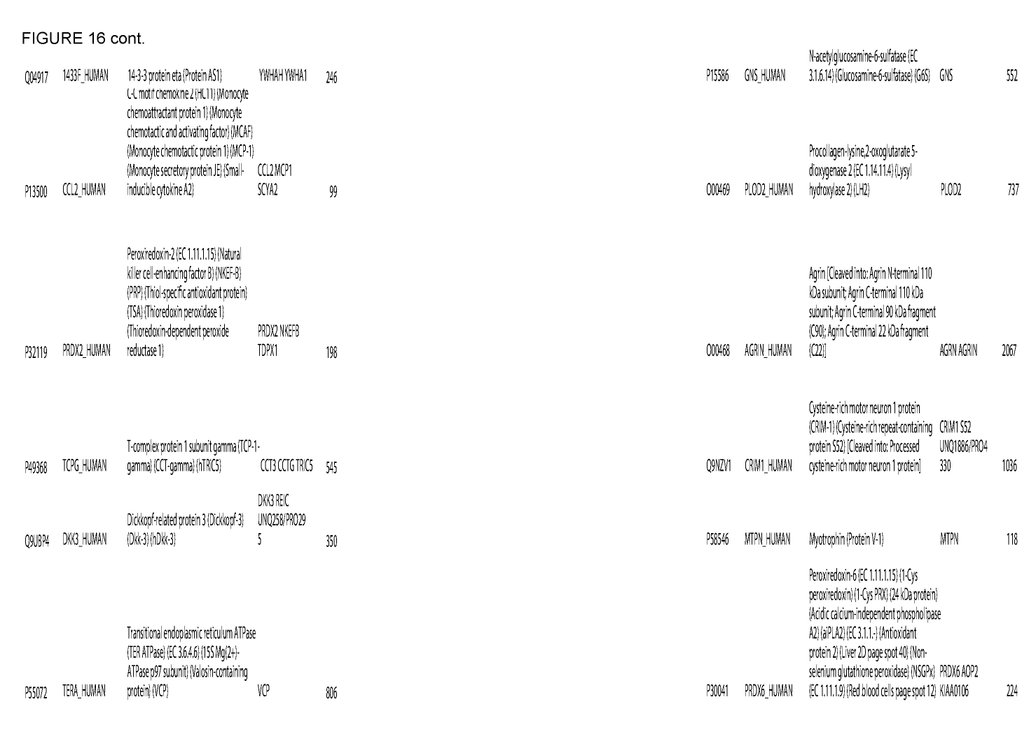

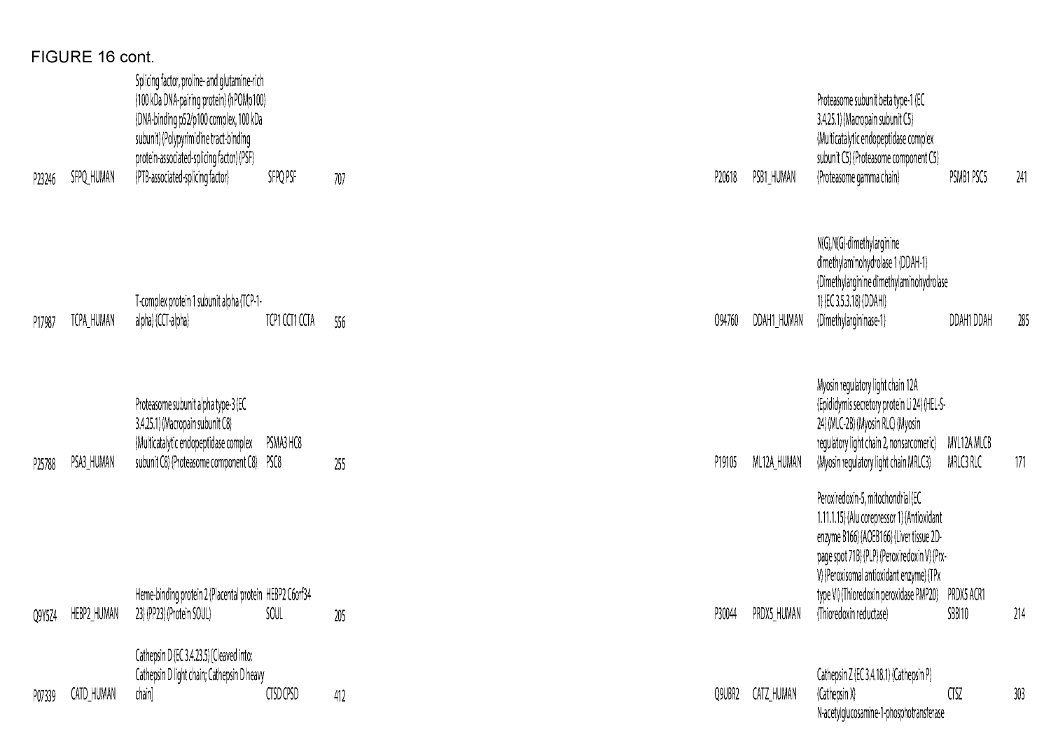

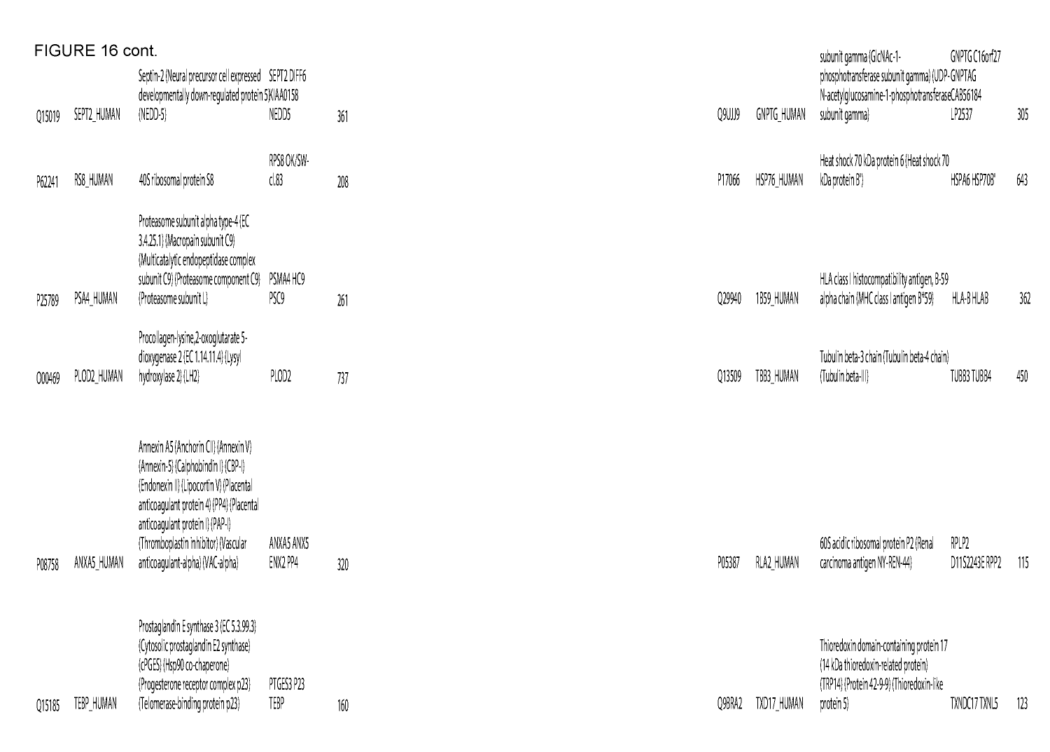

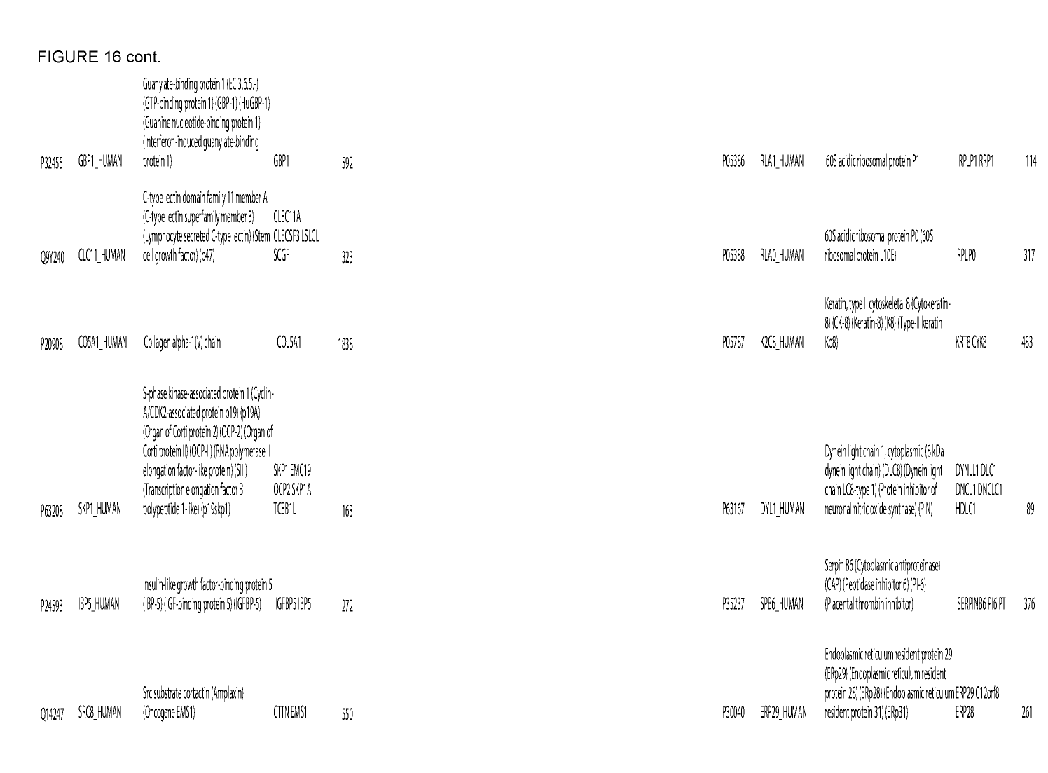

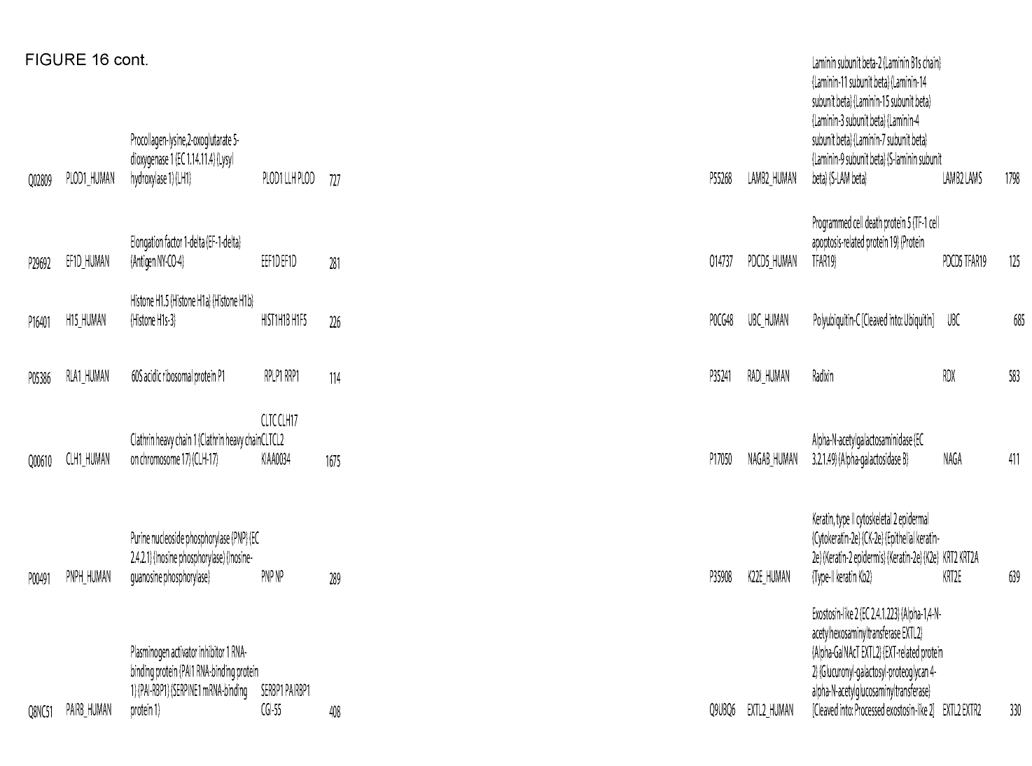

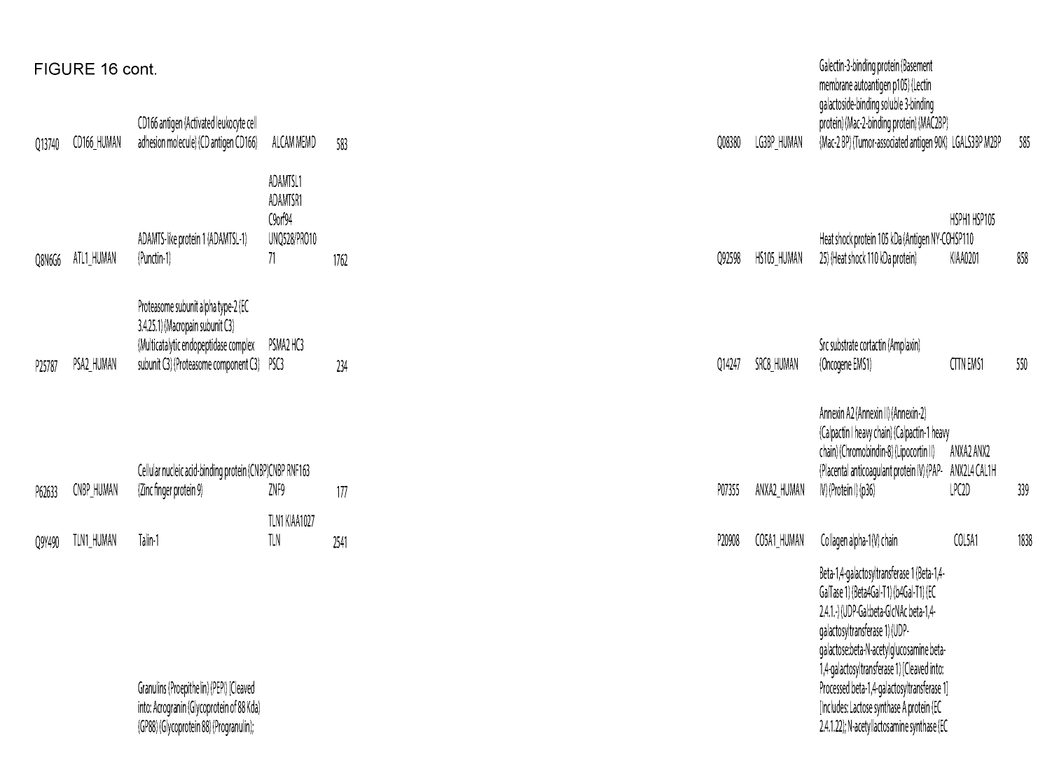

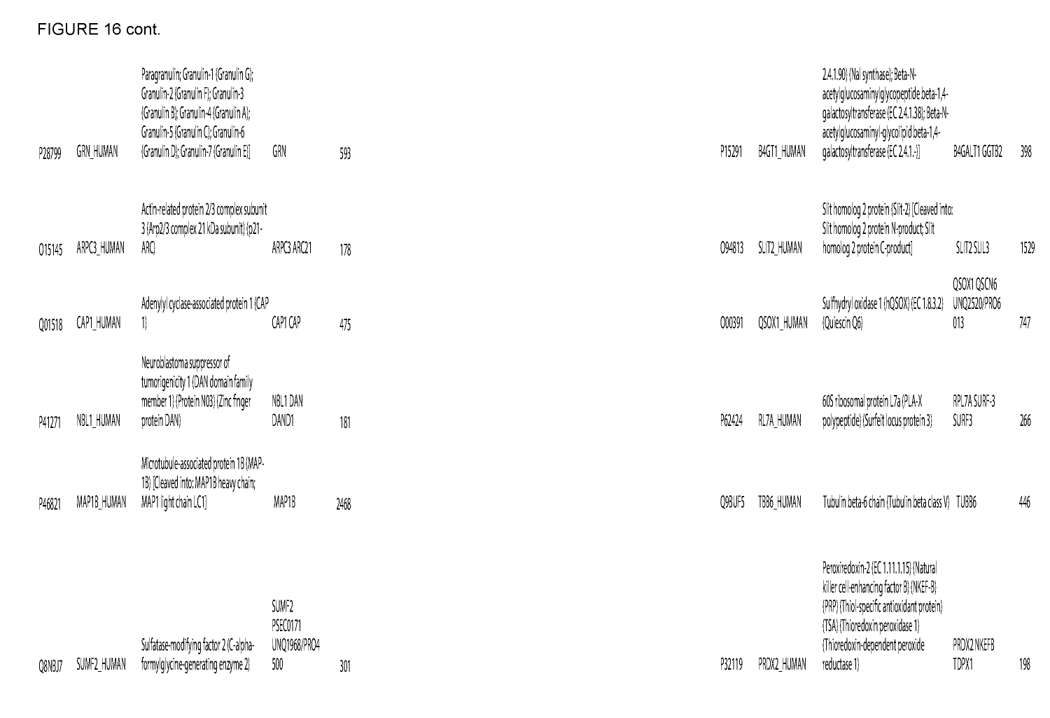

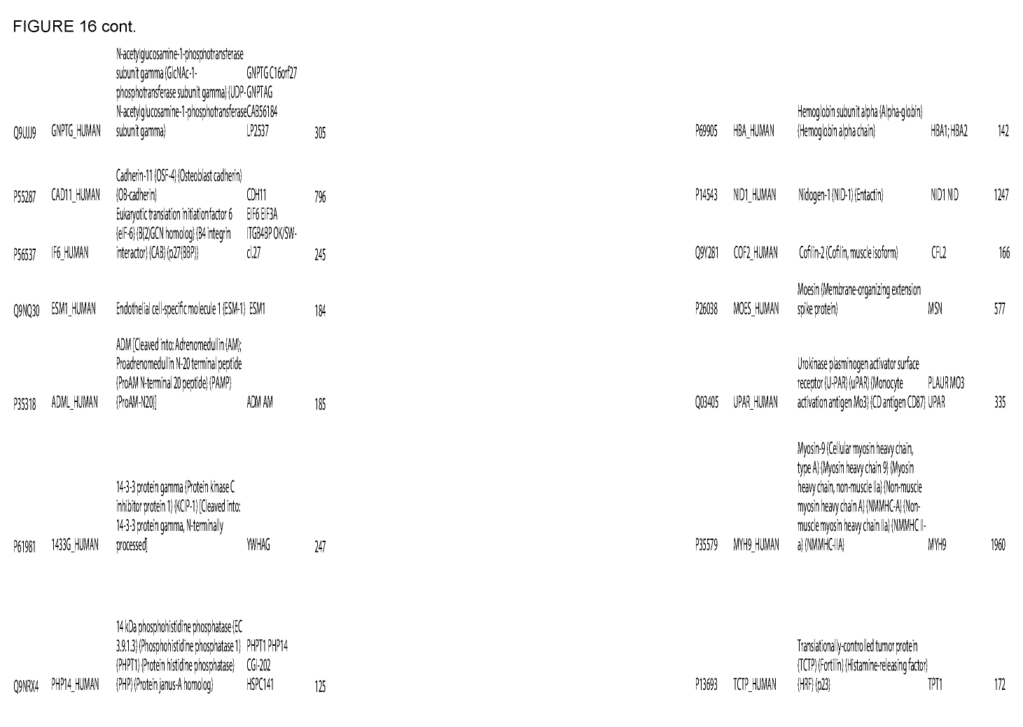

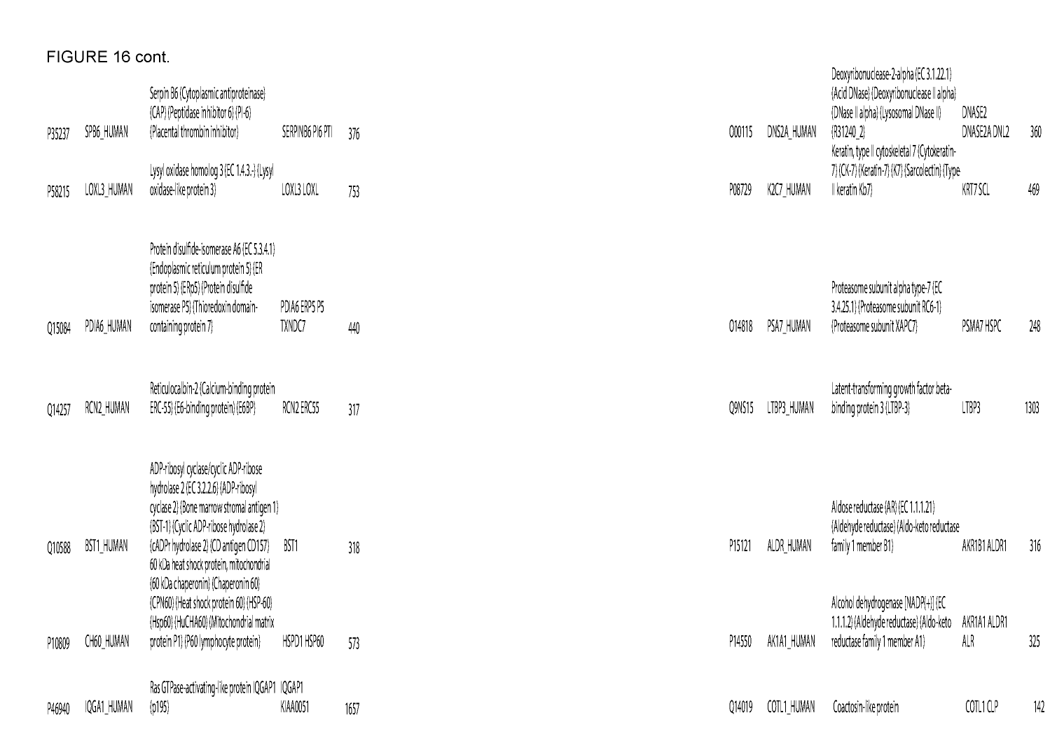

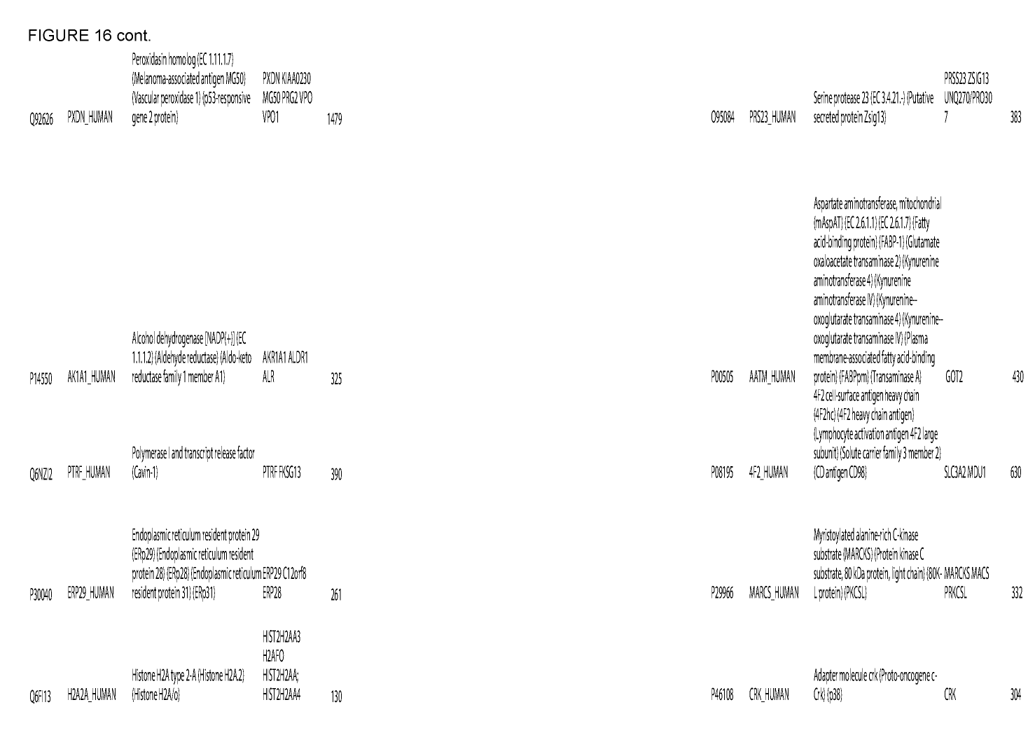

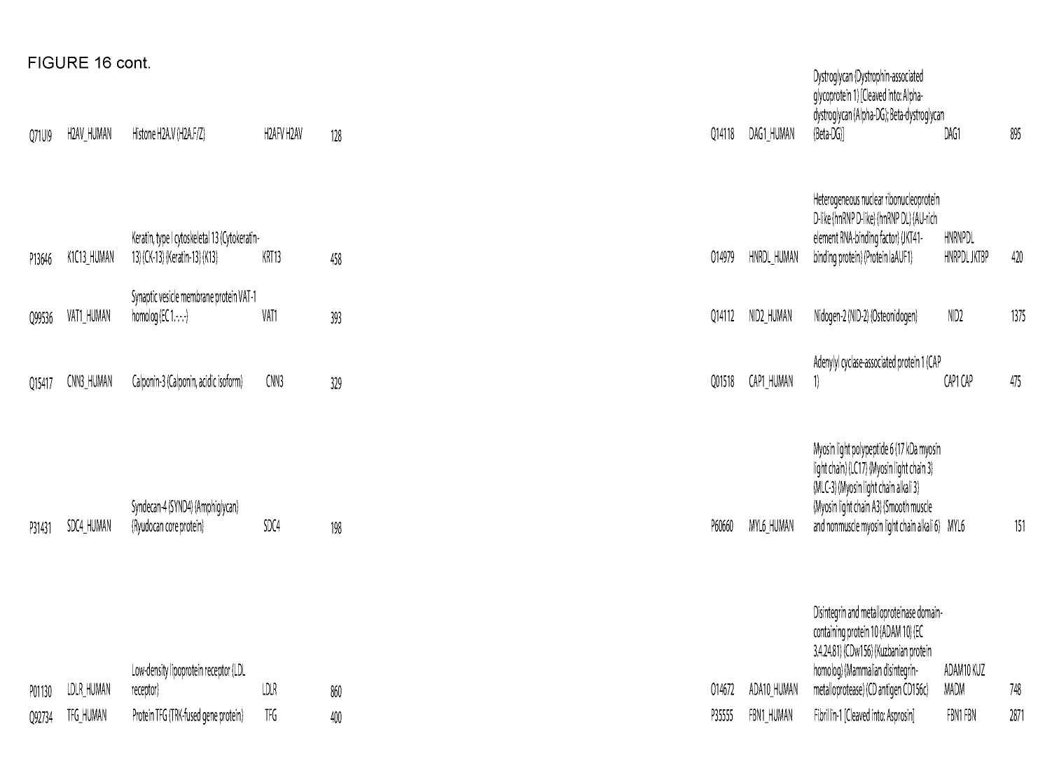

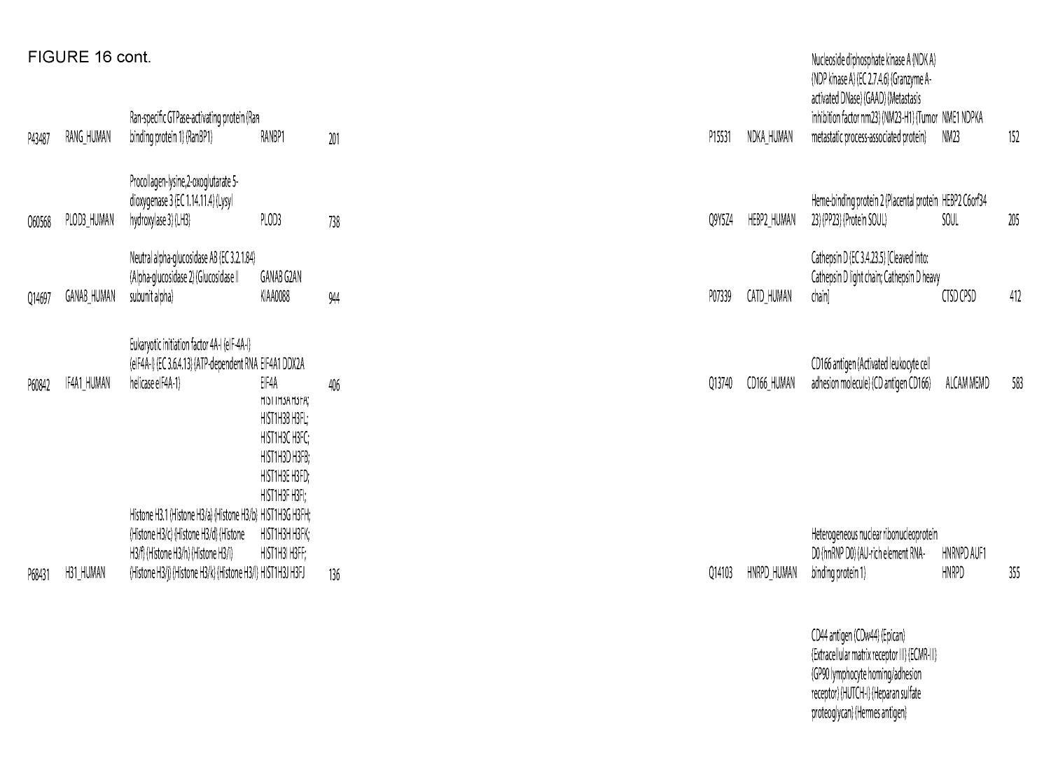

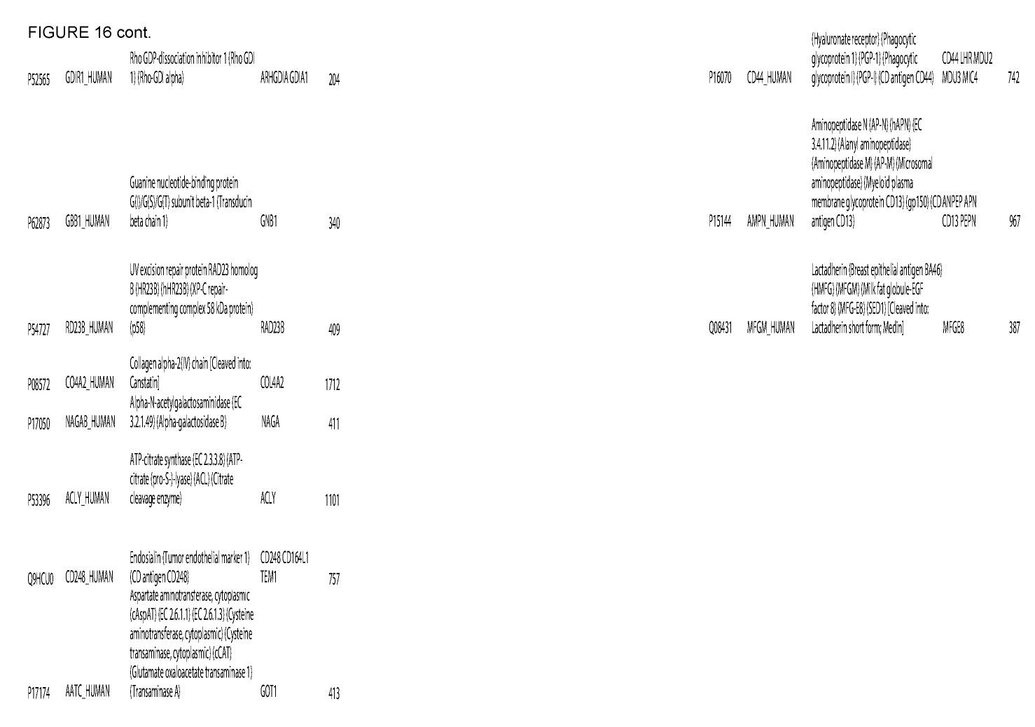

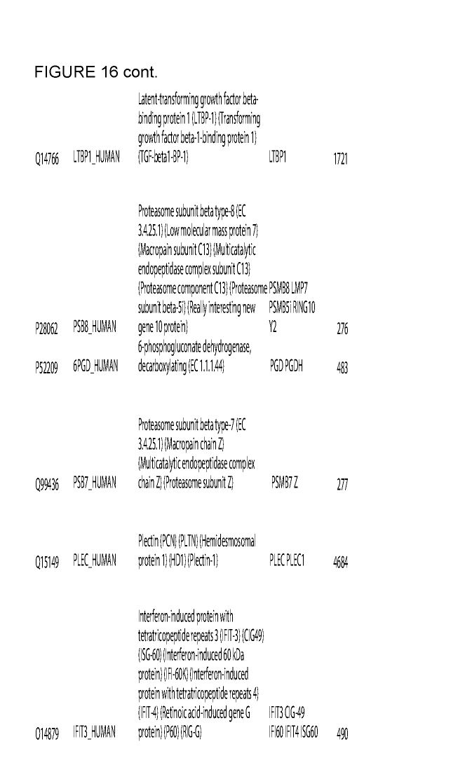

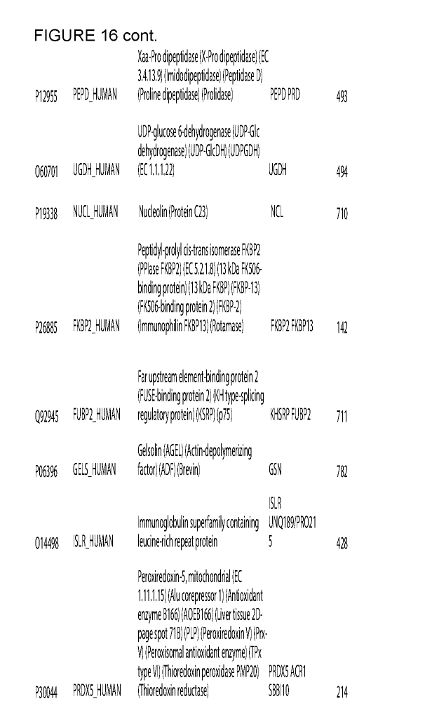

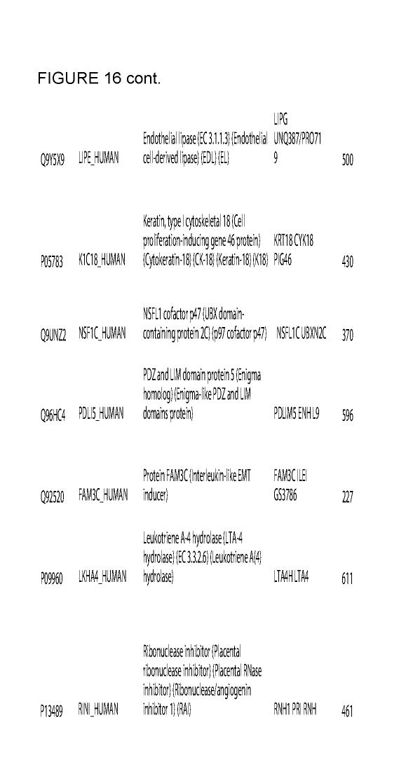

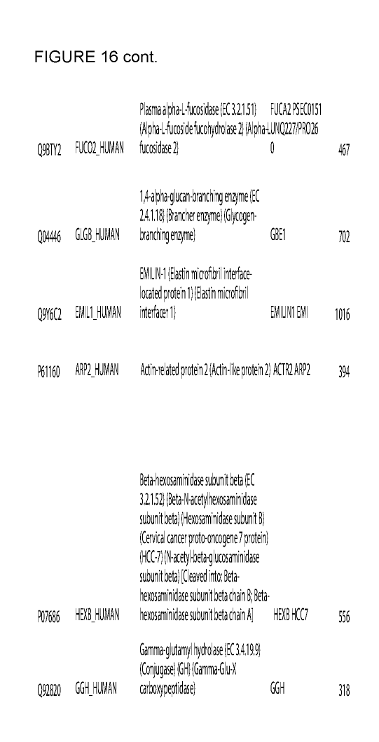

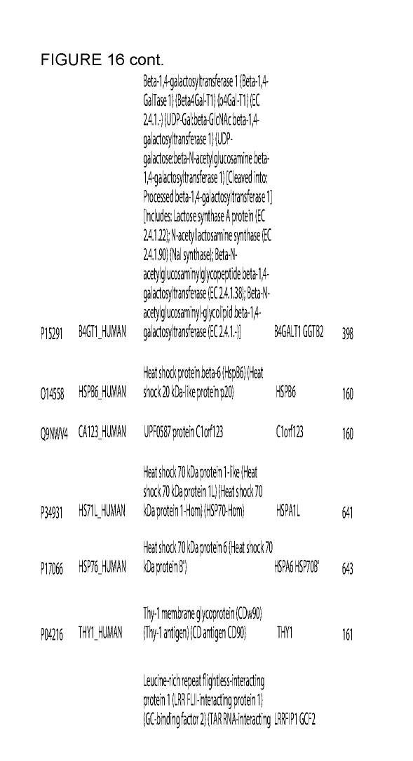

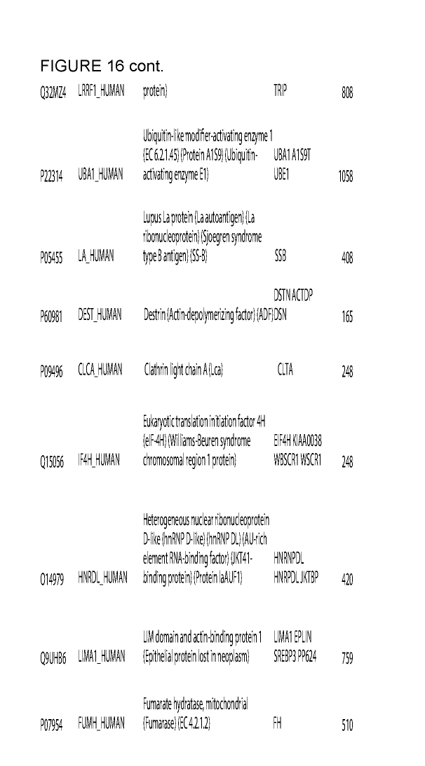

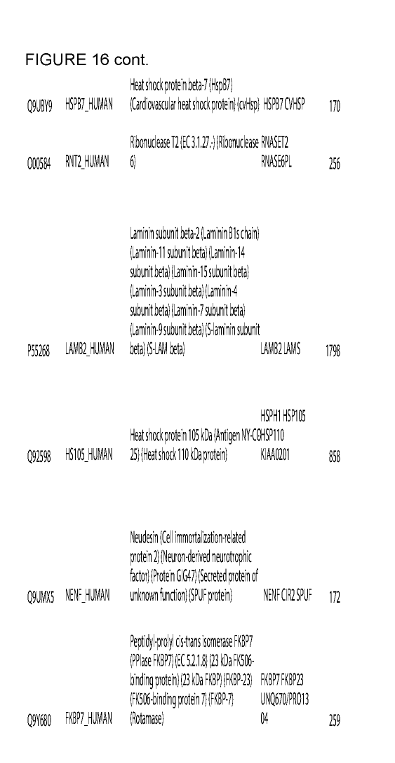

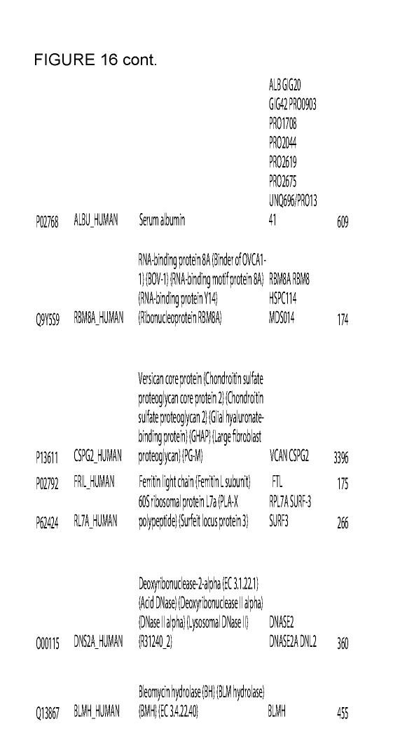

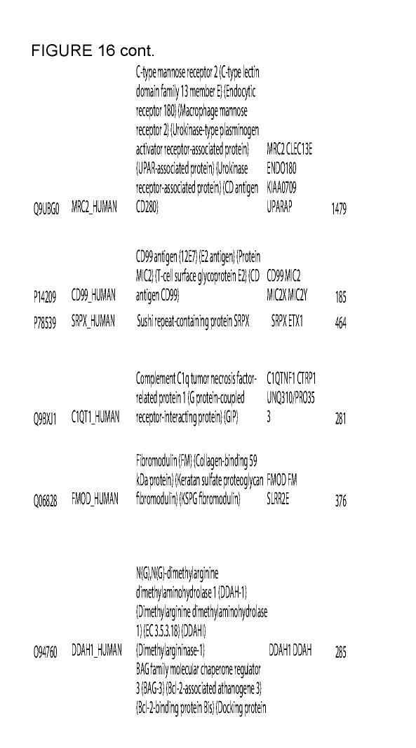

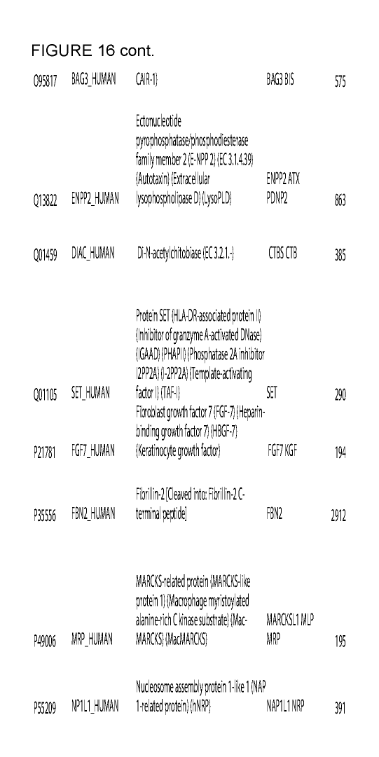

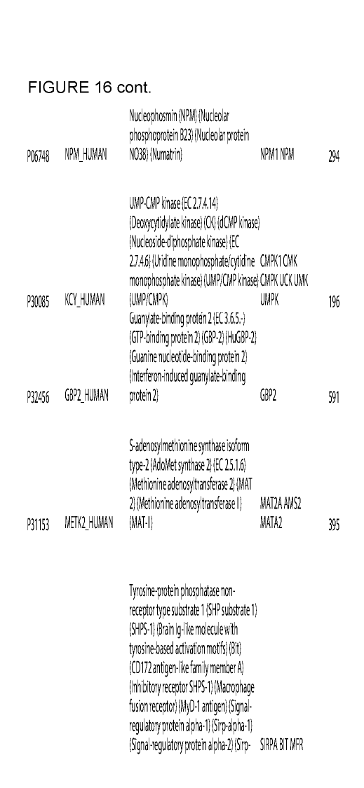

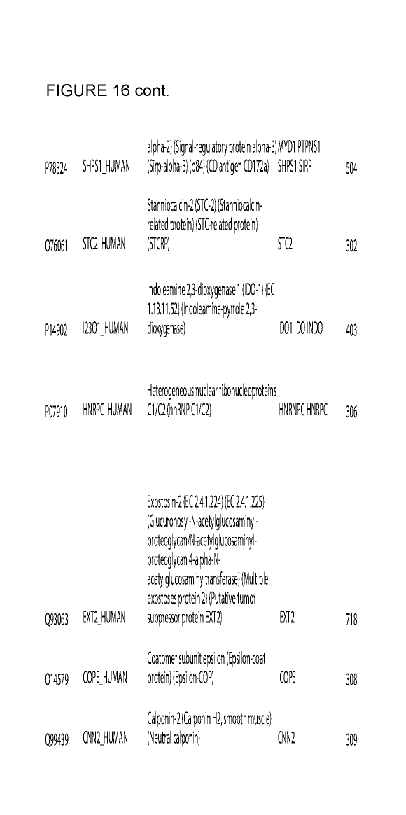

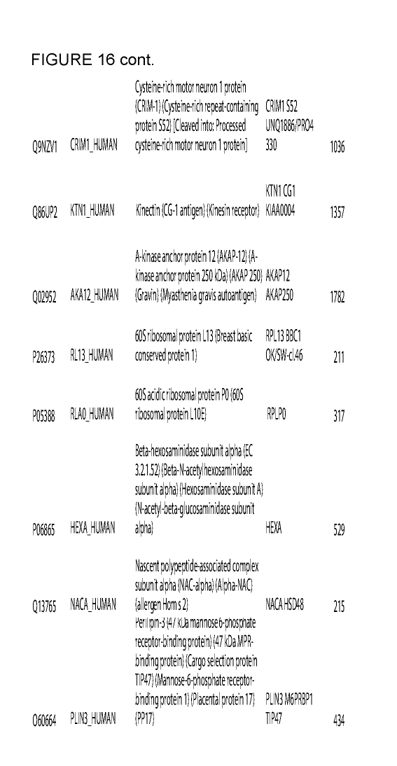

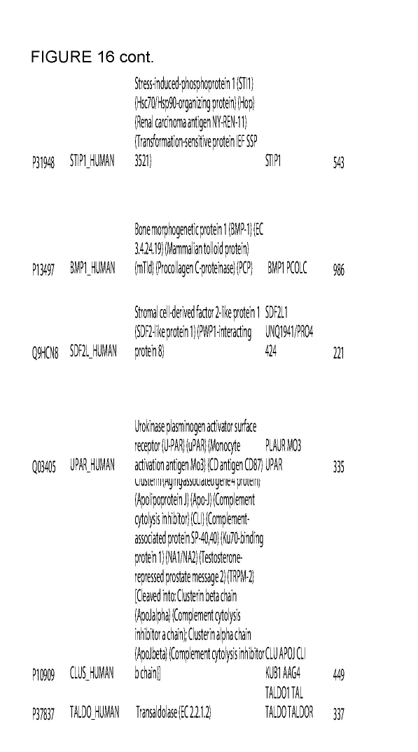

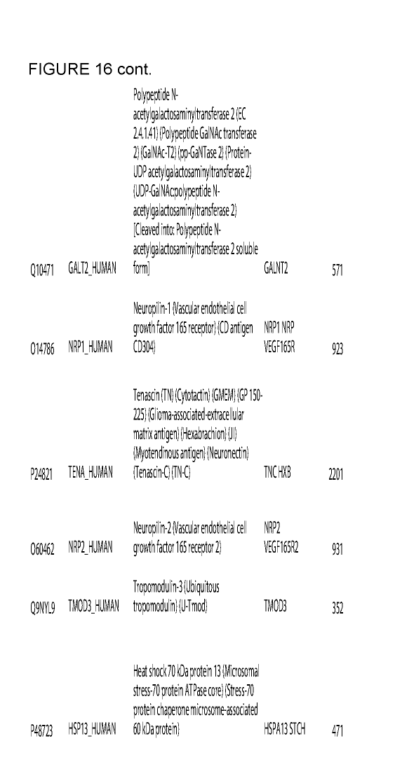

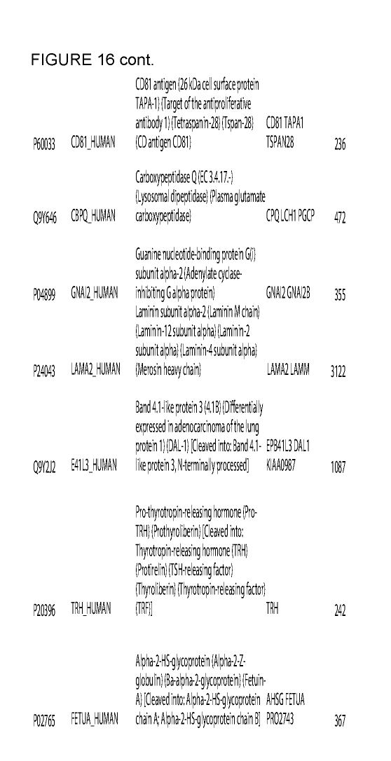

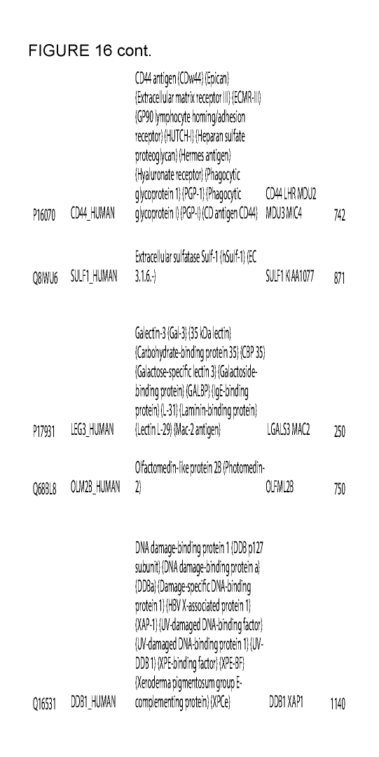

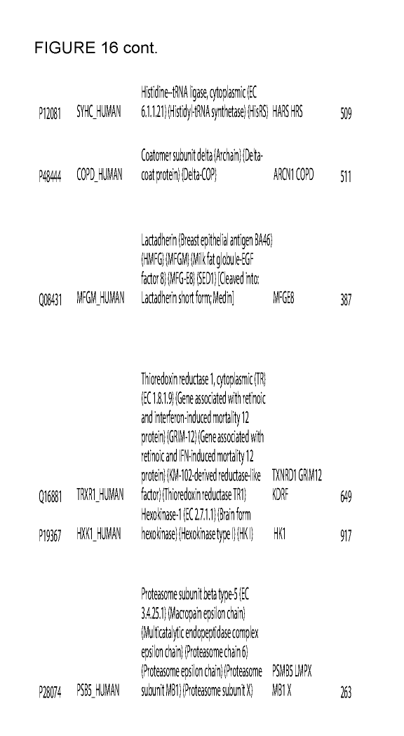

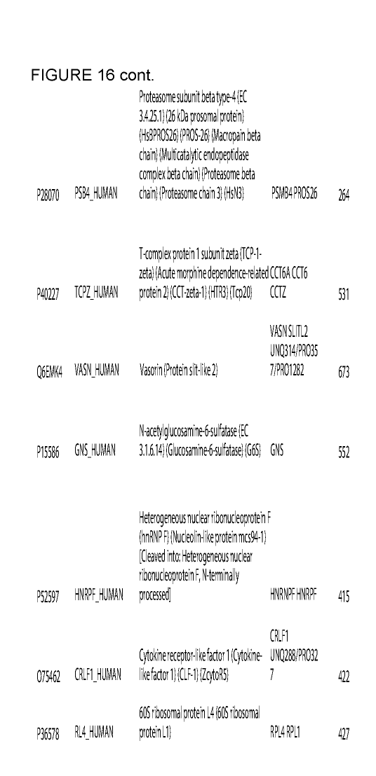

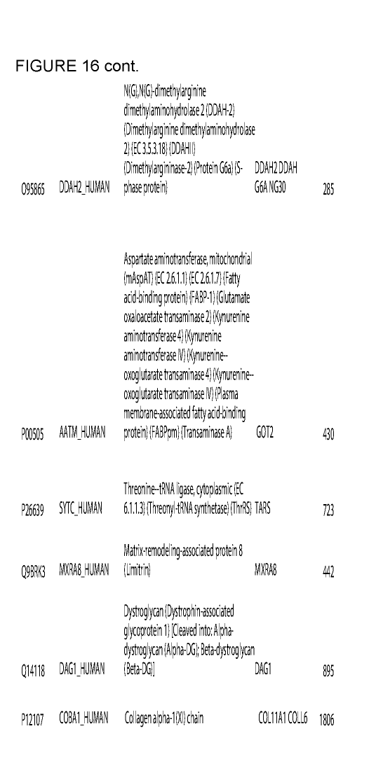

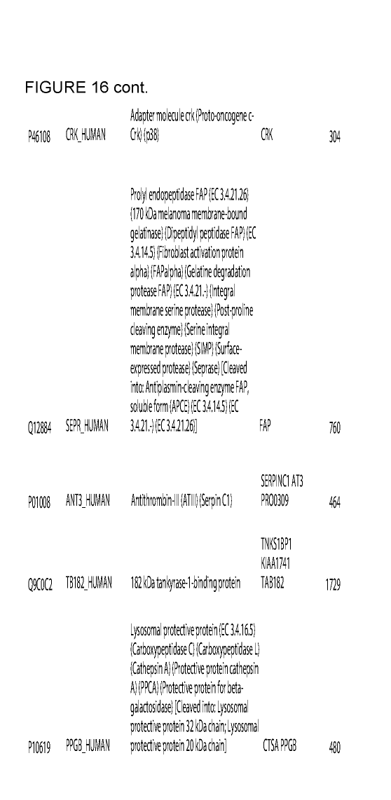

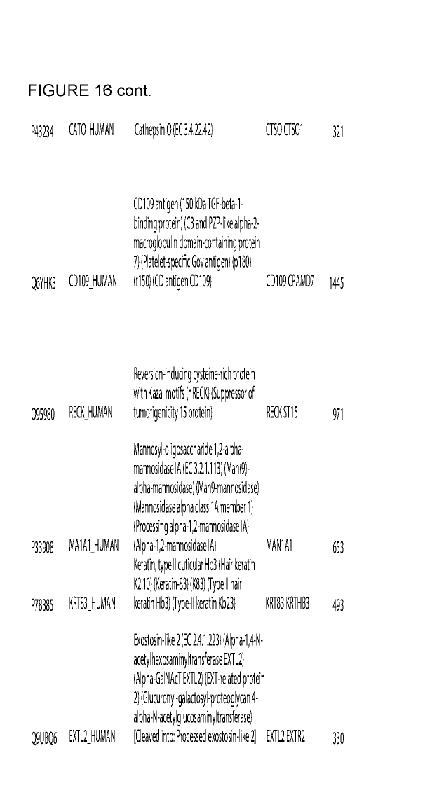

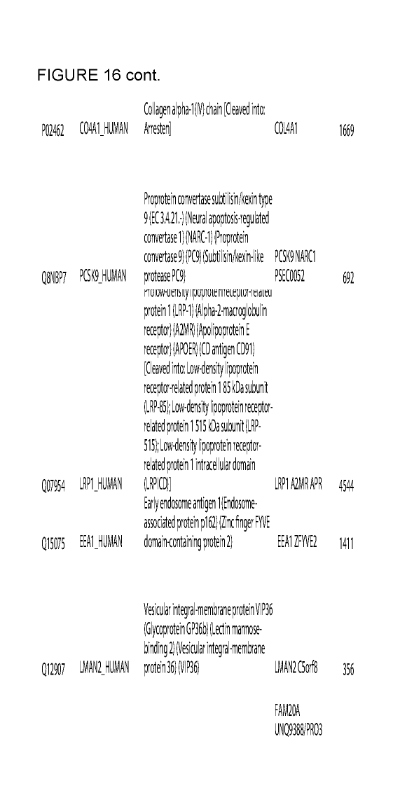

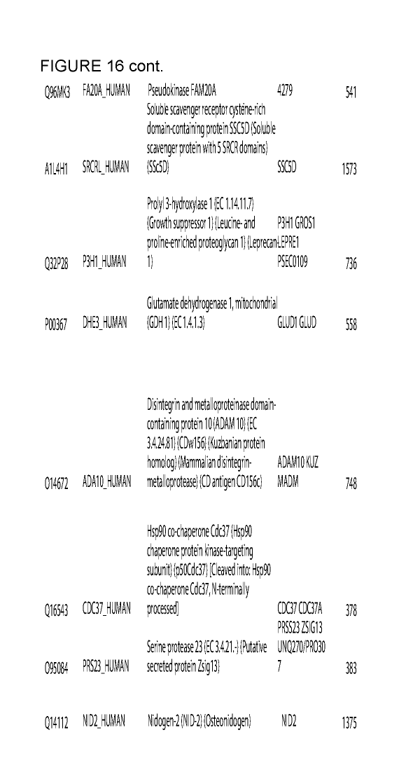

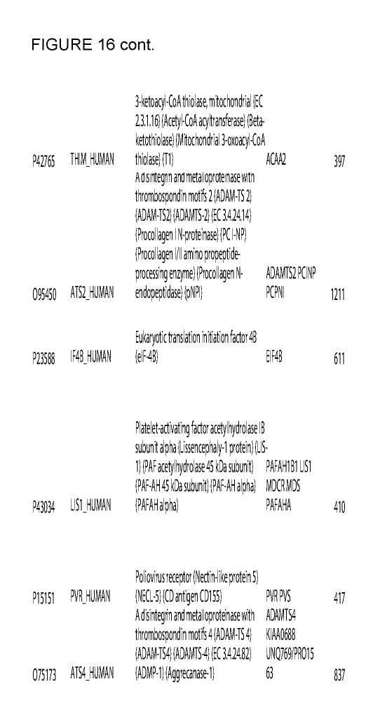

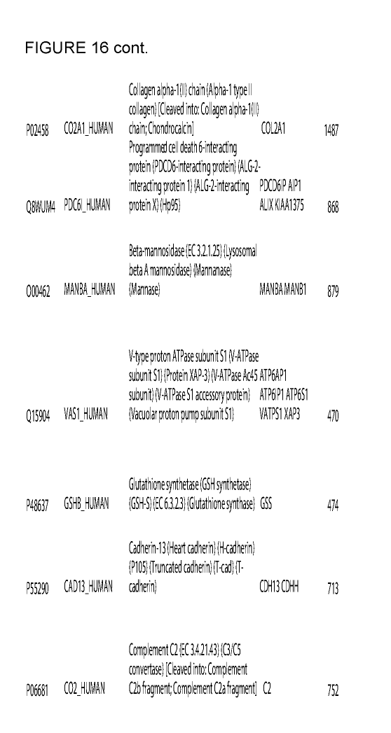

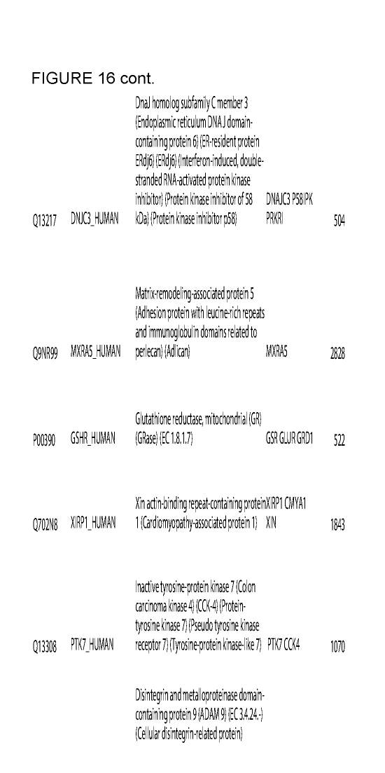

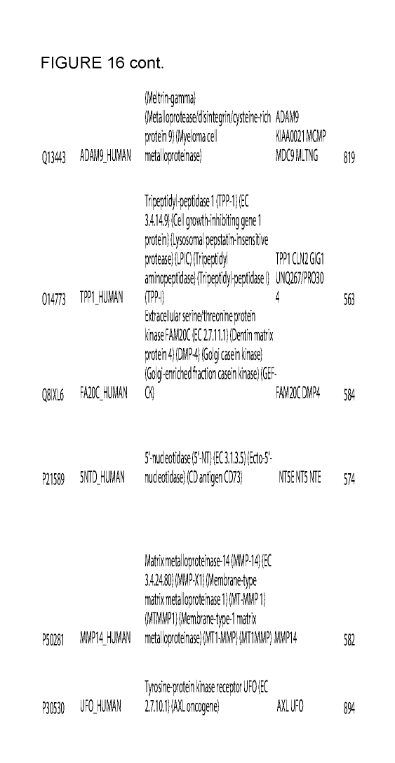

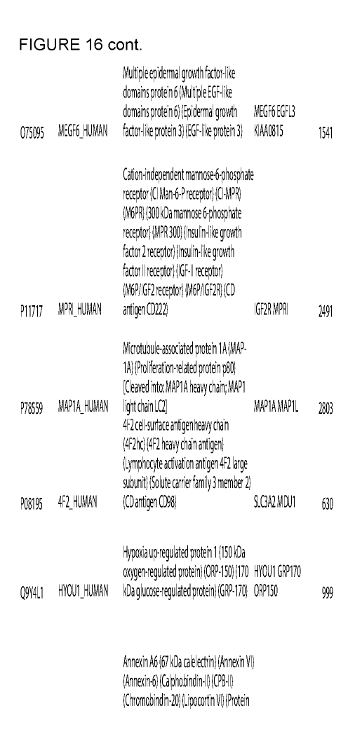

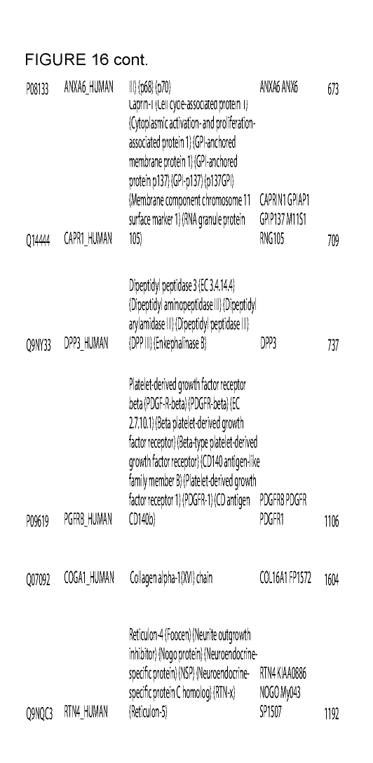

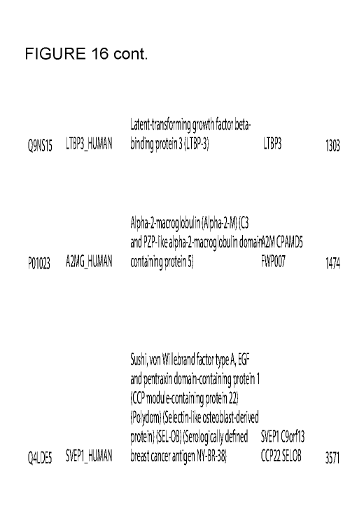

Pre-lyophilized ASC-CM and reconstituted post-lyophilized CC-101 were analyzed by shotgun proteomics. Proteins common to all samples are provided in FIG. 16. As described above, proteins were TCA-precipitated and subjected to LC-MS analysis to identify proteins. FIG. 7E show 100 proteins with high abundance as determined by NSAF values. These include soluble signaling proteins, some of cytokines listed above, and regenerative and anti-inflammatory proteins like TSG-6 (TNFAIP6). Bioinformatic analyses and database searches reveal an overrepresentation of proteins that are known to associate with exosomes. For example, FIG. 7C shows proportional Venn diagrams of proteins identified in ASC-CM or CC-101 compared to proteins in ExoCarta, an online database of putative exosome constituents. Note, that over half of the 100 most frequently identified exosome-associated proteins in ExoCarta are also found in the ASC-CM/CC-101 proteome. FIG. 7D shows the results of functional enrichment analysis (FEA) using DAVID Bioinformatics Resources 6.8. FEA is a computational method to identify classes of genes or proteins that are over-represented in a large set of genes or proteins. Note the abundance and significant over-representation of proteins with gene ontology class “extracellular exosome.”

Identification of miRNAs from ASC-CM Derived Exosomes:

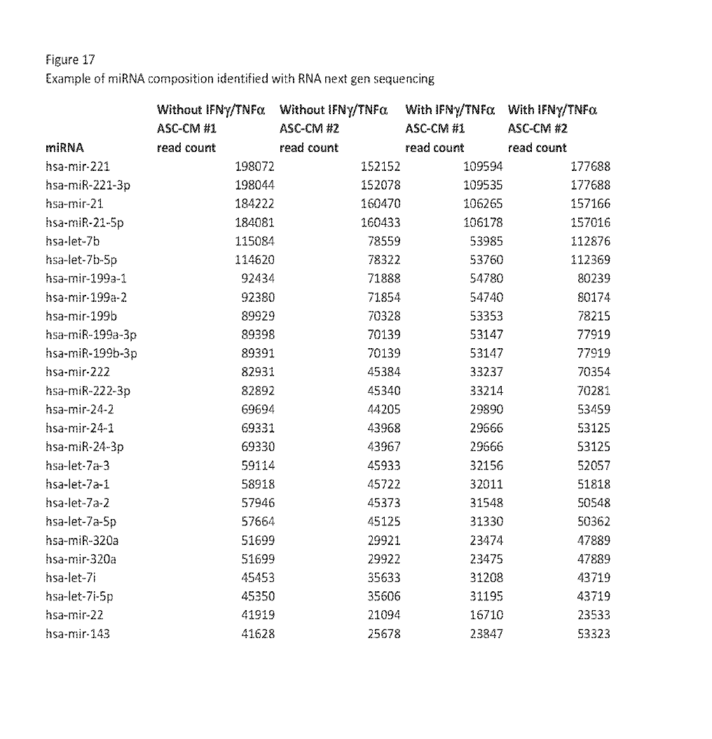

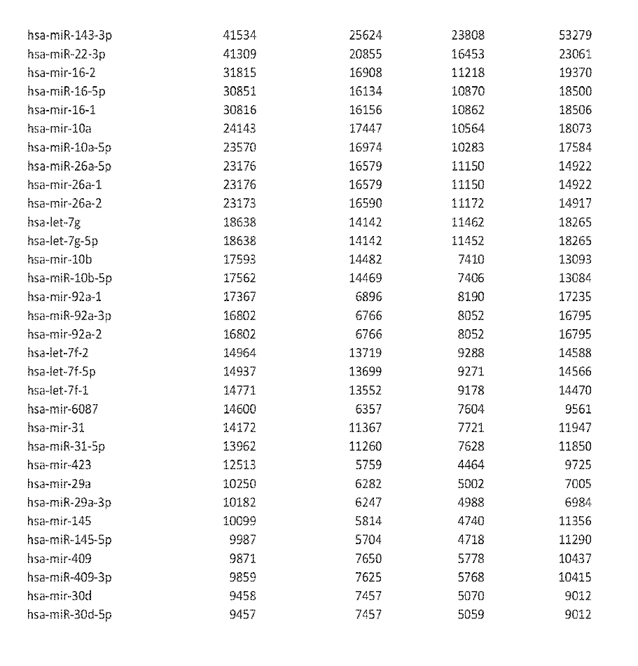

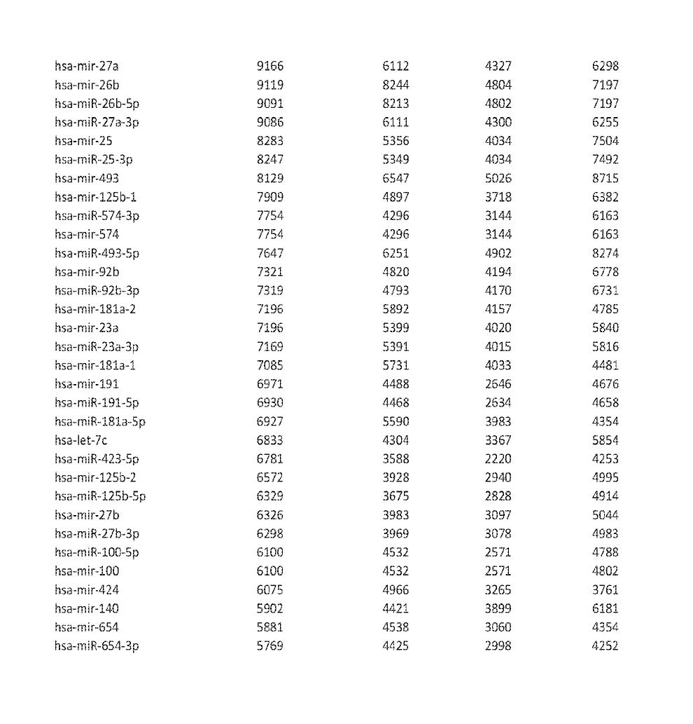

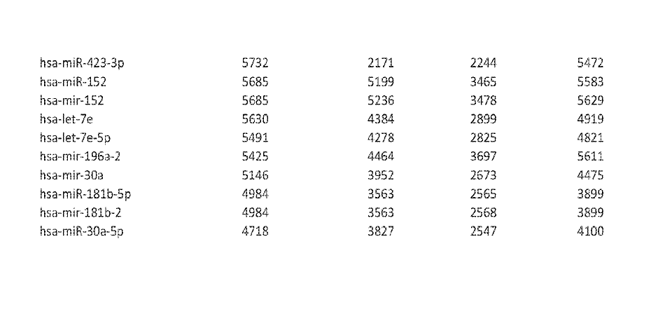

Exosomes were precipitated from ASC-CM using ExoQuick precipitation method (System Biosciences, Palo Alto, Calif.). Exosomal RNA was extracted and quantified by Agilent Bioanalyzer Small RNA Assay. Next Gen Sequencing libraries were prepared and sequenced on an Illumina NextSeq instrument with 1×75 bp single-end reads at a minimum depth of 10 million reads per sample. Raw data was analyzed using Maverix Biomics Platform. Example of top miRNAs identified with RNA next gen sequencing of precipitated exosomes from pre-filtered ASC-CM is provided in FIG. 17.

### Tunable Resistive Pulse Sensing Nanoparticle Analysis:

Concentration and size distribution of EV's was obtained using the qNano system based on tunable resistive pulse sensing (tRPS) technology using NP150 nanopore. Original samples were diluted in PBS-0.03% Tween20 in 1:10 ratio. Concentration was calculated based on polystyrene particles (CPC200) calibration.

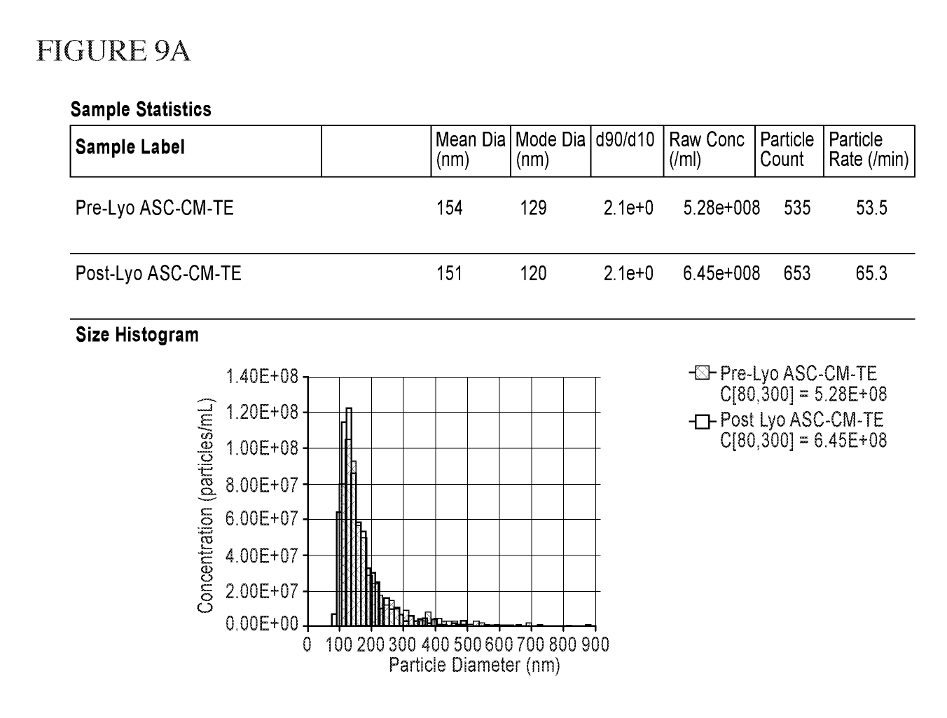

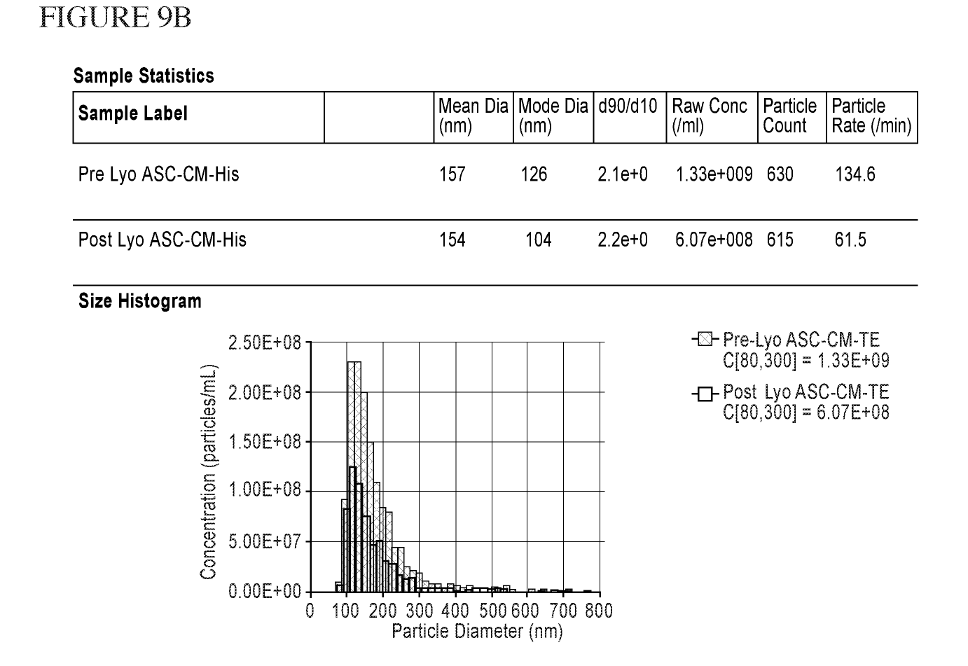

The results shown in FIG. 9A demonstrate that the lyophilization and storage of the product has no detrimental effect on the extracellular vesicles (EVs). Comparison of results for the products in Tris-EDTA buffer pre- and post-lyophilization clearly shows that lyophilization has little to no effect on both the concertation and size distribution of EV's. The product in histidine buffer exhibits preserved EV particle size distribution but a decrease in concentration. (See FIG. 9B)

### Summary:

This study established a Lipoaspirate processing protocol (concentration, incubation time) using GMP grade Liberase and cell culture protocol to produce desired ASC cell population as defined by particular cell surface markers for conditioned media harvest. The priming of ASCs, with IFNγ and TNFα, is important for increasing potency of ASCs and ASC-CM product. For the ASC-CM Harvest, 24 hours or longer (e.g., 48, 72, or more hours) incubation time in serum free medium is used. Depth filtration and Sartobind Q should be performed before TFF in order to remove DNA, viral, and/or protein contaminants. ASC-CM Concentration by TFF was performed using 9× concentration and diafiltration of CM into two different buffers, 25 mM Tris 1mM EDTA pH 8.0 (TE) and 25 mM Histidine pH 8.0 (His). The TFF process does not need to be performed aseptically, as the concentrated product will be filtered post-TFF using a Durapore PVDF filter. To avoid contaminating DNA, the ASC-CM was passed through Sartobind Q Filter and protein was eluted using 500 mM NaCl. DNA removal using a Sartobind Q cartridge can improve both TFF process time and total protein recovery. The lyophilized ASC-CM in TE and His was reconstituted in sterile water with good protein recovery, as evidence by PAGE. While the TE samples would require a much more conservative lyophilization cycle, protein recovery was good. Moreover, the TE formulation performed better than His samples. Following ELISA, ASC-CM was found to contain detectible levels of TIMP1 and VEGF. All samples were stable at 4° C. for at least 10 days.

### Example 2

### Preparation and Testing of Pharmaceutical Compositions Containing a Lyophilized Composition and a Sustained Release Drug Delivery Matrix

A 2 ml solution of about 400 microgram per ml protein concentration of the lyophilized composition prepared according to the methods outlined in Example 1 was prepared and was used as a starting solution for incorporation into a sustained release drug delivery matrix.

Macroscopic matrices were formulated and release profiles were measured from six different matrices. A summary of the matrixes is shown in the table below:

| | | | | | Corresp. |
|---|---|---|---|---|---|
| | | | HSCE | Weight of rel. | HSCE content |
| | Sample ID | Matrix | (payload %) | sample (mg) | (mg) |
| micronized | D-202.1 | Original matrix | 0.037 | Suspension 128.7 | 0.048 |
| | | MgSt:Toco:Exc:HSCE | | Solid matrix 24.2 | |
| | D-202.2 | 77.4:22.6:0:0 | | Suspension 159.7 | 0.059 |
| | | Loaded matrix | | Solid matrix 30.1 | |
| | D-202.3 | MgSt:Toco:H₂O:Exc:HSCE | | Suspension 164.0 | 0.061 |
| | | 14.6:4.3:34.8:46.3:0.04 | | Solid matrix 30.9 | |
| | D-203.1 | Original matrix | 0.030 | Suspension 202.6 | 0.061 |
| | | MgSt:Toco:Exc:HSCE | | Solid matrix 69.7 | |
| | D-203.2 | 90.0:10.0:0:0 | | Suspension 274.3 | 0.082 |
| | | Loaded matrix | | Solid matrix 94.3 | |
| | D-203.3 | MgSt:Toco:Exc:HSCE | | Suspension 198.4 | 0.060 |
| | | 76.5:8.8:3.7:11.0 | | Solid matrix 68.2 | |

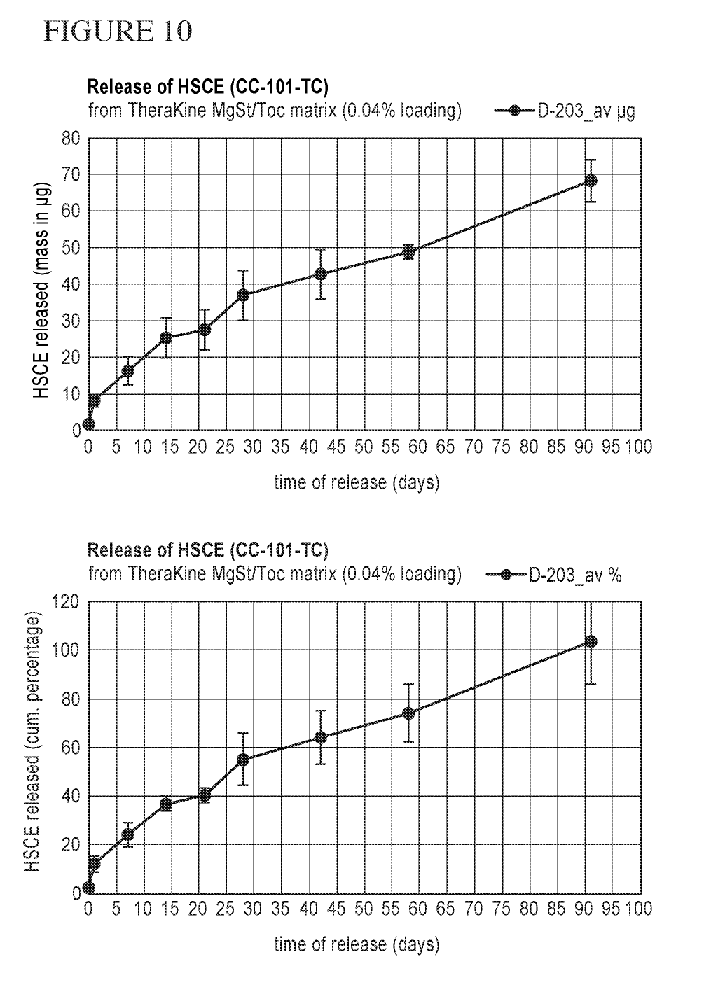

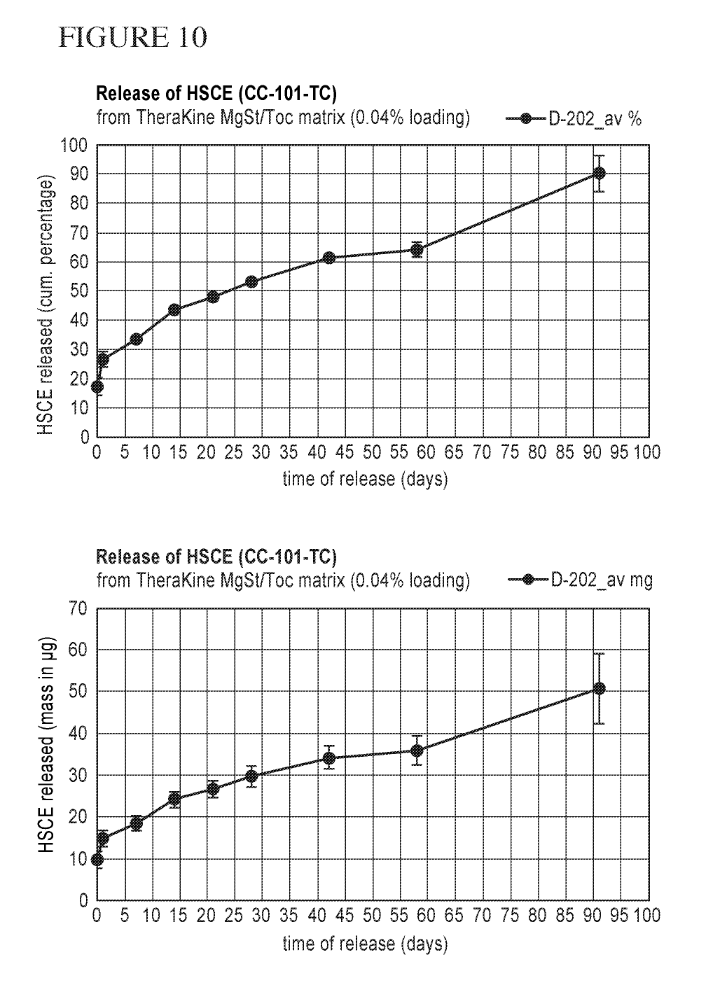

FIG. 10 shows the preliminary release data for the burst and 90 day release measurement points (in percent payload). The observed burst and steady release measurements are in accordance with expectations.

### Example 3

### In Vivo Tolerability Testing of Lyophilized Composition

This study was designed to evaluate the ocular tolerance of CC-101 in non-human primates following intravitreal (IVT) injection to establish a dose for efficacy testing. This was achieved by slit lamp exam, retinal imaging, tonometry, and clinical pathology following repeat escalating dosing.

Test Compound Handling

The vial was sent on dry ice in shipping container maintained below −20° C. over the two day shipping transit time. The vial was then maintained at −20° C. until thawing at room temperature immediately prior to use. At the time of administration, the low dose (64 μg/ml) was formulated by adding 1 mL of 0.9% sterile saline to a 5 mL vial of 15CCT1-150709 CC-101 Histidine. The high dose (128 μg/ml) was formulated by adding 0.5 mL of 0.9% sterile saline to a 5 mL vail of 15CCT1-150709 CC-101 Histidine.

Subject Recruitment

Three adult males, naive to prior drug treatment were selected for study enrollment.

Article Delivery

After confirming good overall health and normal findings by slit lamp exam, fundus imagining and tonometry, the three monkeys recruited to the study were arbitrarily assigned to receive IVT CC-101 or vehicle as indicated in Table below. After achieve mydriasis with 1% cyclopentalate and 10% phenylepharine hydrochloride drops, IVT dosing was performed under ketamine/xylazine sedation (0.2 ml/kg of 100 mg/ml ketamine and 20 mg/ml xylazine) followed by topical 0.5% proparacaine. A lid speculum was placed and the eyes were disinfected with 5% Betadine, which was rinsed off with sterile saline prior to injection. Injections were followed by visual confirmation that there were no injection-associated complications, the topical administration of triple antibiotic ointment (neomycin, polymixin, bacitracin). Post-injection evaluations were then performed in accordance with the exam schedule shown below.

Treatment Assignment

| | | | Treatment | | | | |
|---|---|---|---|---|---|---|---|
| Monkey | Group | Eye | 1 | Route | Dose | Volume | Day |
| K601 | 1 | OU | vehicle | IVT | — | 100 μL | Day 0 |
| K600 | 2 | OU | CC-101 | IVT | vial content/ | 100 μL | Day 0 |
| | | | | | 1 mL saline | | |
| K787 | 2 | OU | CC-101 | IVT | vial content/ | 100 μL | Day 0 |
| | | | | | 1 mL saline | | |
| | | | Treatment | | | | |
| Monkey | Group | Eye | 2 | Route | Dose | Volume | Day |
| K601 | 1 | OU | vehicle | IVT | — | 100 μL | Day 29 |
| K600 | 2 | OU | high dose | IVT | vial content/ | 100 μL | Day 29 |
| | | | CC-101 | | 0.5 mL saline | | |
| K787 | 2 | OU | high dose | IVT | vial content/ | 100 μL | Day 29 |
| | | | CC-101 | | 0.5 mL saline | | |

### Exam Schedule

| Event** | Group | Pre | 0 | 2 | 4 | 7 | 14 | 21 | 29* | 31 | 33 | 36 | 43 | 50 | 71 | Total |
|---|---|---|---|---|---|---|---|---|---|---|---|---|---|---|---|---|
| Dosing | 1 | — | 1 | — | — | — | — | — | 1 | — | — | — | — | — | — | 2 |
| | 2 | — | 2 | — | — | — | — | — | 2 | — | — | — | — | — | — | 4 |
| Slit lamp | 1 | 1 | — | 1 | 1 | 1 | 1 | 1 | 1p | 1 | 1 | 1 | 1 | 1 | 1 | 13 |
| | 2 | 2 | — | 2 | 2 | 2 | 2 | 2 | 2p | 2 | 2 | 2 | 2 | 2 | 2 | 26 |
| Fundus Imaging | 1 | 1 | — | — | — | 1 | 1 | 1 | 1p | — | — | 1 | 1 | 1 | 1 | 9 |
| | 2 | 2 | — | — | — | 2 | 2 | 2 | 2p | — | — | 2 | 2 | 2 | 2 | 18 |
| Tonometty | 1 | 1 | — | 1 | 1 | 1 | 1 | 1 | 1p | 1 | 1 | 1 | 1 | 1 | 1 | 13 |
| | 2 | 2 | — | 2 | 2 | 2 | 2 | 2 | 2p | 2 | 2 | 2 | 2 | 2 | 2 | 26 |
| Clinical Pathology | 1 | 1 | — | — | 1 | — | — | 1 | — | — | 1 | — | — | 1 | — | 5 |
| | 2 | 2 | — | — | 2 | — | — | 2 | — | — | 2 | — | — | 2 | — | 10 |
| Aqueous humor | 1 | — | — | — | — | — | — | 1 | — | — | — | — | — | 1 | — | 2 |
| | 2 | — | — | — | — | — | — | 2 | — | — | — | — | — | 2 | — | 4 |

*Day of 2^{nd} dose at higher concentration
Xₚ: Exams completed prior to dosing
**Numbers in cells indicate the number of animals undergoing a procedure on a given study day

Ophthalmic Exams

Tonometry

Intraocular pressures were measured at baseline and at the indicated post-IVT injection days indicated above. Measurements were performed using a Tono-Vet® tonometer prior to the administration of the mydriatic agent.

Slit Lamp Exams

Eyes were examined by slit lamp exam at baseline and at the indicated post-IVT injection days indicated. Anterior chamber cells, aqueous flare and other ophthalmic findings were graded using a modified McDonald-Shadduck scoring system.

Indirect Ophthalmoscopy

Evaluations of retinal and vitreous inflammation were performed by posterior segment slit lamp exam with a 90-diopter lens. Vitreous cell was scored on a 0 to 5 scale with 0=<5 cells, 1=mild (˜5-10) cells, 2=moderate (˜11-20 cells), 3=marked (˜21-50 cells) and 4=severe (>50 cells) per 1-2 mm slit lamp beam. The vitreous haziness was graded on a scale of 0 to 4 using the Nussenblatt scale with 0=clear vitreous; 1=opacities without obscuration of retinal details; 2=few opacities resulting in mild blurring of posterior details; 3=optic nerve head and retinal vessels significantly blurred but still visible; and 4=dense opacity obscuring the optic nerve head. The presence or absence of retinal infiltrates and hemorrhage, vascular dilation, tortuosity and sheathing, and optic disc edema were also evaluated during ophthalmoscopy.

Fundus Imaging

Color anterior segments and fundus images were acquired at baseline and on scheduling imaging days using a Topcon TRC-50EX retinal camera with Canon 6D digital imaging hardware and New Vision Fundus Image Analysis System software. Fundus photographs were reviewed to evaluate any signs of adverse effects and associated changes. No quantitative scoring was applied.

Blood Collection

Blood (9 mL) was collected at baseline and day 4, 21, 33 and 50 post-injection. 3 ml of whole blood was anti-coagulated with K₂EDTA and shipped on ice to Ross University School of Veterinary Medicine Diagnostic Services for complete blood count (CBC) with differential. Another 3 ml of blood was transferred to citrate centrifuge tube, inverted 3× and centrifuged at 3000 rpm for 10 minutes at 4° C. and the resulting plasma (1.5 ml) transferred to a labeled cryotubes and flash frozen prior to shipment in a nitrogen vapor shipper to Antech GLP for coagulation profiles. Serum was prepared by incubation of the blood in centrifuge tubes without clot activators for 1 hour at room temperature to allow clotting followed by centrifugation at 4° C. at 3000 rpm for 10 minutes. Serum aliquots were transferred to pre-labeled cryotubes and flash frozen prior to shipment in nitrogen vapor shipper to Antech GLP Super for clinical chemistry profiles.

Aqueous Collection

Aqueous humor (˜0.05 mL) was sampled using a 0.3 mL syringe with a 31 gauge after anterior chamber paracentesis OU at baseline and at the indicated post-IVT injection days indicated, after prepping the eye as indicated for IVT injection. Aqueous aliquots were transferred to pre-labeled cryotubes and flash frozen, stored in −80° C. prior to shipment to the Sponsor in a nitrogen vapor shipper and on dry ice.

### Clinical Observations

General wellbeing will be confirmed twice daily by cage side observations. Body weights were collected at the times at which animals are sedated for ophthalmic exams.

### Criteria for Evaluation

Primary Endpoints
Slit lamp exams
Fundus imaging
Aqueous samples
Plasma samples
CBC samples
Serum samples

Secondary Endpoints
Clinical observations

### Results

### Clinical Exams

The monkey was evaluated by physical exam at baseline and at each ophthalmic observation interval for general health, including body weight and integrity of integument, thorax and abdomen. All physical exam findings were within normal limits. Ophthalmic exams revealed that both eyes receiving intravitreal vehicle injections tolerated the procedure well with minimal injection-associated complications. The same was true of low dose CC-101, with only mild, transient iris hyperemia (K600 Day 7) and keratic precipitate (K600 Day 14). Administration of higher concentration CC-101, however, resulting in more persisting ocular inflammation, again manifesting as mild keratic precipitate (K600 & K787 Day 31 & 33) and anterior chamber cell (K787 Day 31, K600 & K787 Day 33), accompanied by mild iris hyperemia (K600 Day 31-43, K787 Day 36 & 43), lens capsule cell (K787 Day 31 & 33) and vitreous cell, (K787 Day 33 -43). All signs of inflammation following high dose CC-101 resolved by day 21 post-dosing.

### Imaging

Anterior segment and fundus images of the vehicle treated eyes (K601 OU) remained within normal limits at all exam intervals with the exception of a reduced response to mydriatic at Day 29 in K601 OD, resulting in diminished fundus image quality. Reduced pupil response to mydriatic and consequent diminished fundus image quality was more prevalent in the CC-101 treated eyes, occurring in K600 OS on day 14, 21 and 29, and to a lesser degree in K600 OD over the same time points, and in K787 OU on day 7, 29, 36 and 43, though the anterior and posterior poles otherwise remained within normal limits.

### Clinical Pathology

Clinical pathology parameters remained stable throughout the study.

### Safety

Data generated in this study was primarily designed to assess the tolerability of CC-101 Histidine after ocular injection. Tolerability was evaluated via slit lamp exam, tonometry and fundus imaging, which revealed minor inflammation at higher CC-101 concentrations. No adverse systemic effects of the IVT injection were observed by daily cage side exams. Animals were returned to general housing at the close of the study in good health.

### Conclusion:

Drug induced ocular inflammation is observed and well-recognized with intraocular IVT drug treatment. This study was designed to assess the ocular tolerance of CC-101 Histidine to establish a dose for efficacy testing in the African green monkey. The initially evaluated dose of CC-101, in which the lyophilized contents of a dosing vial were suspended in 1 mL 0.9% saline was well tolerated following intravitreal injection, with minimal transient inflammatory changes detected by slit lamp exam that resolved by three weeks post-dosing.

This finding provided rationale for exploring a 2× higher dose, which resulted in more persistent and consistent mild inflammatory signs by slit lamp exam, that again, fully resolved by three weeks post-dosing. Given that the higher CC-101 dose was evaluated in the same eyes/animals as the lower dose, the possibility that the observed inflammatory response reflected an immune response to repeat CC-101 exposure rather than to the higher dose itself, cannot be excluded.

As a human cell derived drug product, it would be anticipated that CC-101 would contain antigenic peptide fractions that might contribute to such an adaptive immune response. Until chronicity of dosing is controlled for in subsequent study designs a no-observed-adverse-effect-level (NOAEL) cannot be clearly defined, however, these data do indicate that the 1× single dose CC-101 is well tolerated and would represent an appropriate dose for pharmacodynamics evaluations in African green monkey test system.

### Example 4

### In Vivo Traumatic Brain Injury and Vision Deficit Study

Traumatic brain injury frequently leads to progressing vision problems resulting in blindness. Inflammation due to microglial polarization following blast injury may play a vital role in the development of visual defects. In this study, it was assessed whether CC-101 or mesenchymal stem cells derived from adipose tissue (adipose-derived stem cells ADSC) can limit retinal tissue damage from blast injury and improve visual function through direct cell-to-cell contact or cell-independent paracrine signaling.

### Methods:

### Blast Injury Model

The overpressure air blast is delivered by a small horizontally mounted air cannon system consisting of a modified paintball gun (Invert Mini, Empire Paintball), pressurized air tank, and x-y table.

12 weeks old C57B1/6 mice were subjected to 50-psi air pulse limited to a 7.5 mm diameter area on the left side of the head, mid-cranial area.

### CC-101 Treatment

Within lhr of blast injury, 1000 human ADSC labeled with maurocalcine-cy5 peptide or 1 μL CC-101 (64 μg/ml) (N=8 mice/group) were intravitreally delivered into both eyes. Blast only and no-blast control mice received saline.

### In Vivo Live Visual Function Experiments

After 3 and 6 weeks post blast/injection, visual function experiments were assessed by Optokinetic Tracking (OKT) for acuity and contrast sensitivity by the standard procedures using OptoMotry (Cerebral Mechanics).

Fluorescein Angiography was performed to measure vascular permeability. Mice were anesthetized with Ketamine/Dexdomitor cocktail, and the eyes were dilated with tropicamide. About 75 μl of Sodium fluorescein (2.5 mg/ml) was injected i.p and imaged the retina (only LE) within 30-60seconds of injection. RE was imaged subsequently (average 2-3 min after i.p injection), Micron IV Retinal microscope (Phoenix Research Lab) was used to capture bright field, Cy5 fluorescence (where possible) and Green fluorescence using the appropriate filters. Snap shots were taken from videos.

### GFAP Immunohistochemistry

At the end of the study, animals were euthanized. Eyes were enucleated at 6 weeks post ADSC or ADSC-CM (CC-101) injections, fixed in 4% paraformaldehyde in PBS, cryoprotected in 30% sucrose overnight at 4° C. Afterward, eyes were embedded in Optimal cutting temperature (OCT) compound and cryosectioned into 10 μm sections. GFAP immunohistochemical analysis was performed by an investigator blinded to the study groups. Briefly, retinal sections from near the optic nerve head (ONH) were washed with 1X PBS to remove the OCT compound, boiled in citrate buffer, pH 6.0 for antigen retrieval, and blocked in goat blocking buffer (10% goat serum/5% BSA/0.5% Triton X-100 in PBS) for 30 min at room temperature. To assess for gliosis, sections were incubated overnight with GFAP primary antibodies (ThermoFisher, 1:250) at 4° C. in a humidified chamber. Next day, sections were washed 3 times with 1X PBS and incubated with a 1:500 goat anti-mouse IgG conjugated to AlexaFluor488, and DAPI (both ThermoFisher) to stain nuclei for 1.5 hr at room temperature, then washed with 1X PBS. Finally, slides were mounted with Prolong Diamond media (ThermoFisher) and let dry overnight at room temperature in darkness. For each slide, one section was kept as negative control without primary antibody. Digital images were captured from regions intermediate to the ONH and the ora serrata from three retinal sections approximately 20-100 μm apart using a Zeiss 710 laser scanning confocal microscope and quantification of pixel intensities of each antigen was computed using ImageJ analysis software.

### Results:

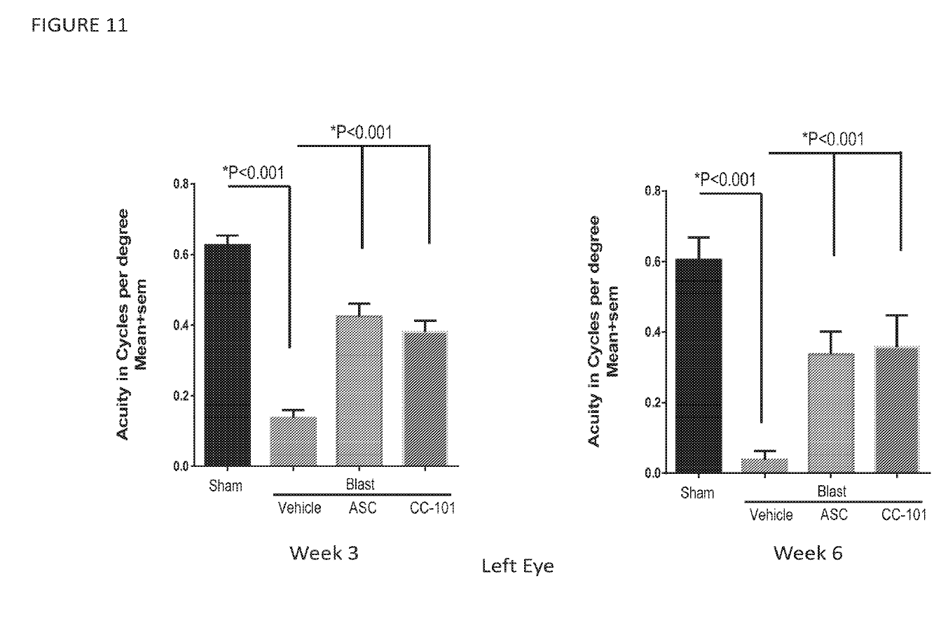

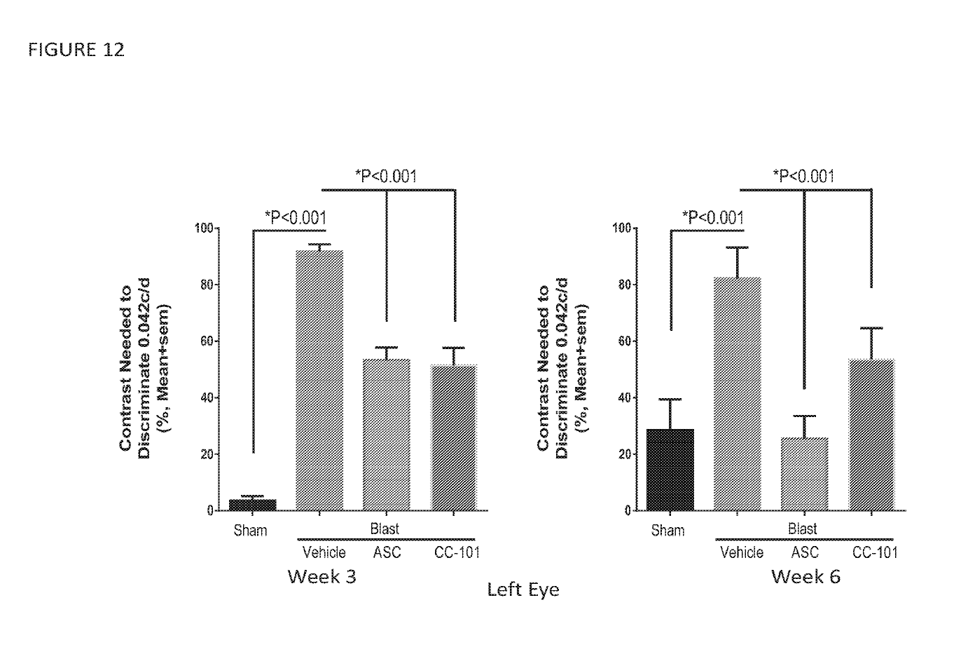

As shown in FIG. 11, intravitreal injection of CC-101 improved visual acuity in blast mice at 3 weeks and the protective effective sustained at 6 weeks. Likewise, intravitreal injection of CC-101 also improved contrast sensitivity. (See FIG. 12). Intravitreal injection of ADSC as well demonstrated similar effect as CC-101.

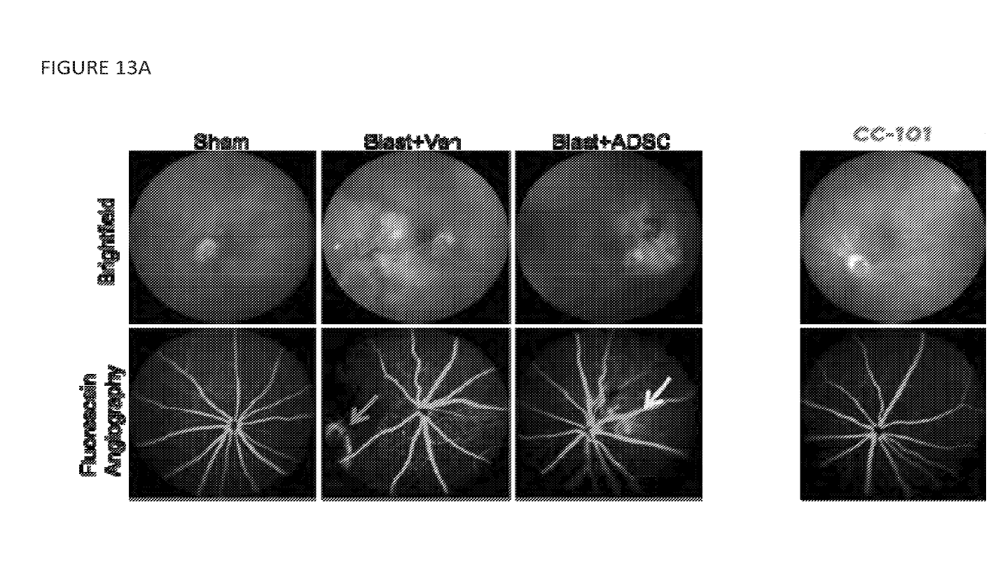

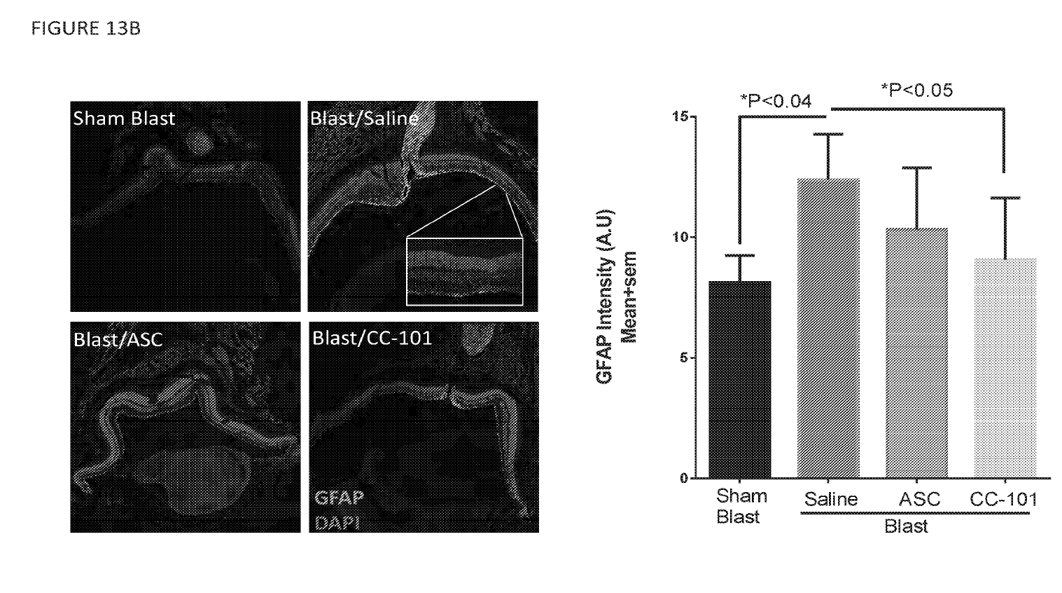

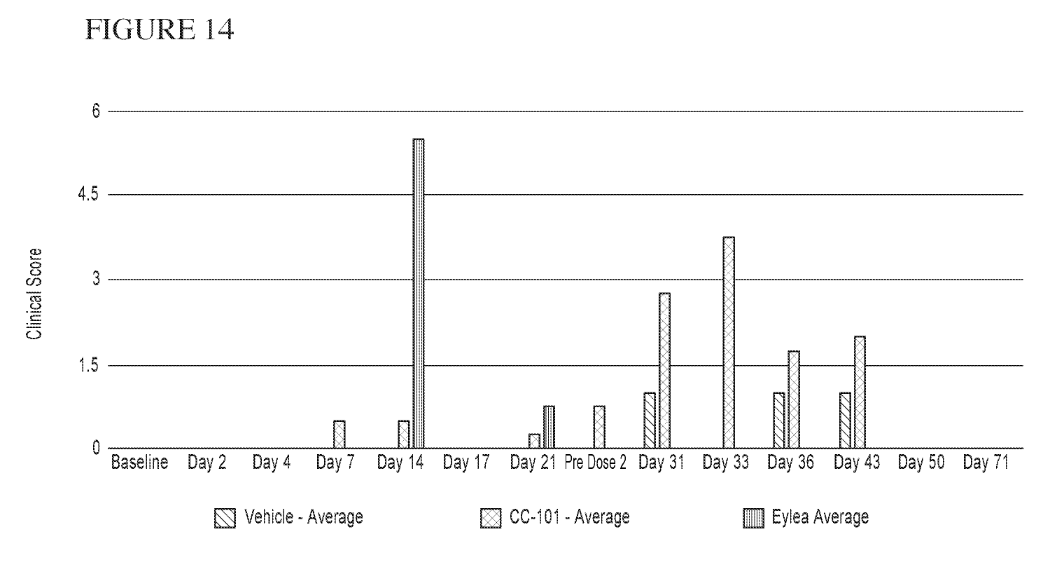

Intravitreal administration of ADSC and/or CC-101 improved vascular leakage as observed with brightfield and fluorescein angiography imaging. (See FIG. 13A). The focal blast mild TBI model showed extensive lesions (possibly hyper proliferation of RPE) in the retina accompanied by fluorescein leakage (microvascular damage), which were near completely absent in the animals that received CC-101. Interestingly, ADSC that were labeled with cy-5 were found to be associated specifically with lesions. Immunohistological analysis of CC-101 treated animals also showed significantly less GFAP in regions intermediate to the ONH and the ora serrata (See FIG. 13B).

### Conclusion:

The findings suggest that CC-101 as well as ADSC improve visual deficits of the blast injury through their anti-inflammatory properties on activated pro-inflammatory microglia and retinal endothelial cells. Although additional studies are warranted, visual rescue from TBI appears to function through cell-independent paracrine signaling. Considering the similarities in the observed therapeutic effects of ADSCs and CC-101, a shelf-stable regenerative therapy for immediate delivery at the time of injury may provide a practical and cost effective solution against the traumatic effects of blast injuries to the retina.

Blast injury reproducibly shows lesions and vascular leakage. CC-101 animals that were blast injured showed a significant normal appearance and no leakage.

### Example 5

### In Vitro Vascular Permeability Assay

50×10³ HRMVEC cells were cultured with 250 μl CSC complete media (10% serum; Cell Systems Inc) on the coated (with attachment factor, Thermo Fisher Scientific) upper chamber (0.4 μm polycarbonate transwell, Corning, Inc.) and 500 μl CSC complete media (10% serum) was filled in the bottom chamber at 37° C., 5% CO₂.

After 48 hours, the upper chamber was exposed to Staurosporine (ST, 1μm) with and without CC-101 (diluted in 1:1 ratio with fresh media to maintain 10% serum). Bottom wells were replaced with 500 μl of media with 10% Serum.

After 2 hours of treatment, media from the upper chamber was removed and 100 μl of 4 kDa-FITC dextran (5 mg/ml, Sigma-Aldrich) in Ca/Mg free DPBS was added. After 1 hour, 100 μl of the media from the bottom well was collected and fluorescence was measured using a plate reader (485 nm extrication and 520 emission). The amount of fluorescence measured gives the amount of FITC dextran leakage through the HRMVEC cells present on the upper chamber-transwell.

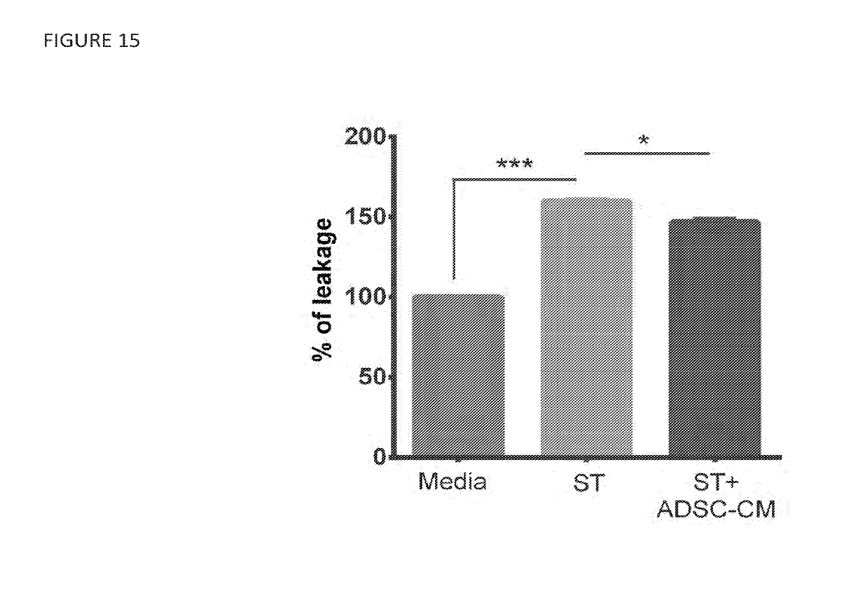

As shown in FIG. 15, human retinal endothelial cells in the inserts incubated with ST showed a significant two-fold increase in fluorescence. On the other hand, cells incubated with CC-101 showed a significant reduction in fluorescence. Data shown is from a single experiment performed in triplicate (***p<0.01; *p<0.05).

### EQUIVALENTS

The details of one or more embodiments of the invention are set forth in the accompanying description above. Although any methods and materials similar or equivalent to those described herein can be used in the practice or testing of the present invention, the preferred methods and materials are now described.

The foregoing description has been presented only for the purposes of illustration and is not intended to limit the invention to the precise form disclosed, but by the claims appended hereto.

1. A lyophilized composition comprising
a) a concentrated, cell-free conditioned medium comprising the secretome of cultured adipose cells, wherein the adipose cells comprise at least one adipose stem cell (ASC) and wherein at least 90% of the cultured adipose cells express a pericyte marker; and
b) an effective amount of a lyophilizing agent.

2. The lyophilized composition of claim 1, wherein the pericyte marker is selected from the group consisting of CD140b, CD73, CD90, and CD105 and wherein the cultured adipose cells are negative for CD45, CD14, CD19, HLA-DR, and CD31.

3. The lyophilized composition of claim 2, wherein at least 95% of the cultured adipose cells express the pericyte marker.

4. The lyophilized composition of claim 1, wherein the lyophilizing agent is sucrose.

5. The lyophilized composition of claim 1, wherein the composition additionally comprises an effective amount of a buffer for filtration.

6. The lyophilized composition of claim 5, wherein the buffer for filtration comprises Tris-EDTA or histidine.

7. The lyophilized composition of claim 6, wherein the effective amount of Tris-EDTA is about 25 nM Tris and about 1 mM EDTA.

8. The lyophilized composition of claim 1, wherein the adipose cells are obtained following liposuction.

9. The lyophilized composition of claim 8, wherein the adipose cells are obtained from a female.

10. The lyophilized composition of claim 1, wherein the composition is shelf-stable at a temperature between about 20 and 35° C. for a period of at least 3 months.

11. The lyophilized composition of claim 1, wherein the composition is non-immunogenic.

12. The lyophilized composition of claim 1, wherein the secretome comprises a therapeutically effective amount of one or more regenerative or anti-inflammatory factors.

13. The lyophilized composition of claim 12, wherein the one or more regenerative or anti-inflammatory factors are selected from the group consisting of cytokines, chemokines, growth factors, enzymes, microRNAs, phospholipids, polysaccharides, and any combinations thereof

14. The lyophilized composition of claim 13, wherein the one or more regenerative or anti-inflammatory factors i) are separate from extracellular vesicles or ii) are bound within or on the surface of the extracellular vesicles secreted by the ASCs.

15. (canceled)

16. The lyophilized composition of claim 12, wherein the at least one ASC has been cultured under conditions that increase the expression of the one or more regenerative or anti-inflammatory factors.

17. The lyophilized composition of claim 16, wherein the at least one ASC has been cultured in the presence of exogenously added amounts of IFNγ and TNFα.

18. The lyophilized composition of claim 17, wherein the expression of one or more of Growth-regulated alpha protein (CXCL1), interleukin-6 (IL6), interleukin-8 (IL-8), C-C motif chemokine 2 (CCL2), C-C motif chemokine 8 (CCL8), C-C motif chemokine 5 (CCL5), C-X-C motif chemokine 10 (CXCL10), or Tumor necrosis factor receptor superfamily member 11B (TNFRSF11B) is increased.

19. The lyophilized composition of claim 17, wherein the secretome comprises one more miRNAs selected from the group consisting of hsa-miR-221/222, hsa-miR-199, hsa-miR-22, hsa-miR-16, and hsa-miR-26.

20. The lyophilized composition of claim 12, wherein the composition comprises between 0.05-1.5 mg/ml total protein.

21. The lyophilized composition of claim 1, wherein the composition comprises between 1×10⁸ and 9×10¹¹ extracellular vesicles

22. A pharmaceutical composition comprising an effective amount of the lyophilized composition of claim 1 and a sustained release drug delivery matrix.

23. The pharmaceutical composition of claim 22, wherein the sustained release drug delivery matrix is biodegradable, biocompatible, or both biodegradable and biocompatible.

24. The pharmaceutical composition of claim 22, wherein the pharmaceutical composition releases therapeutically effective amounts of one or more regenerative and anti-inflammatory factors from the secretome of the adipose cells for a period of up to 6 months.

25. The pharmaceutical composition of claim 24, wherein the one or more regenerative or anti-inflammatory factors are selected from the group consisting of cytokines, chemokines, growth factors, enzymes, microRNAs, phospholipids, polysaccharides, and any combinations thereof.

26. The pharmaceutical composition of claim 25, wherein the regenerative or anti-inflammatory factors stimulate tissue regeneration, neurovascular repair, or both tissue regeneration and neurovascular repair.

27. The pharmaceutical composition of claim 25, wherein the one or more regenerative or anti-inflammatory factors are i) separate from extracellular vesicles or ii) are bound within or on the surface of extracellular vesicles secreted by the ASCs.

28. (canceled)

29. The pharmaceutical composition of claim 22, wherein the at least one ASC has been cultured under conditions that increase the expression of the one or more regenerative or anti-inflammatory factors.

30. The pharmaceutical composition of claim 29, wherein the at least one ASC has been cultured in the presence of exogenously added amounts of IFNγ and TNFα.

31. The pharmaceutical composition of claim 30, wherein the expression of one or more of Growth-regulated alpha protein (CXCL1), interleukin-6 (IL6), interleukin-8 (IL-8), C-C motif chemokine 2 (CCL2), C-C motif chemokine 8 (CCL8), C-C motif chemokine 5 (CCL5), C-X-C motif chemokine 10 (CXCL10), or Tumor necrosis factor receptor superfamily member 11B (TNFRSF11B) is increased.

32. The pharmaceutical composition of claim 25, wherein the composition comprises between 0.05 mg/ml to 1.5 mg/ml of total protein.

33. The pharmaceutical composition of claim 22, wherein the sustained release drug delivery matrix is selected from the group consisting of a gel, a paste-like composition, a semi-solid composition, and a microparticulate composition.

34. The pharmaceutical composition of claim 33, wherein the sustained release drug delivery matrix is mechanically formed through macroscopic processing.

35. The pharmaceutical composition of claim 34, wherein the sustained release drug delivery matrix does not cause any chemical or biological changes to the lyophilized composition.

36. The pharmaceutical composition of claim 33, wherein the sustained release drug delivery matrix comprises a hydrophobic matrix.

37. The pharmaceutical composition of claim 36, wherein the hydrophobic matrix comprises one or more hydrophobic excipients selected from the group consisting of magnesium stearate, magnesium palmitate, fatty acid salts, cetyl palmitate, fatty acid salts, plant oils, fatty acid esters, tocopherols, and combinations thereof.

38. The pharmaceutical composition of claim 37, wherein the hydrophobic matrix comprises magnesium stearate and tocopherol.

39. The pharmaceutical composition of claim 36, wherein the hydrophobic matrix comprises at least a hydrophobic solid component and a hydrophobic liquid component.

40. The pharmaceutical composition of claim 39,
wherein the hydrophobic solid component is selected from the group consisting of waxes, fruit wax, carnauba wax, bees wax, waxy alcohols, plant waxes, soybean waxes, synthetic waxes, triglycerides, lipids, long-chain fatty acids and their salts, magnesium palmitate, esters of long-chain fatty acids, long-chain alcohols, waxy alcohols, oxethylated plant oils, and oxethylated fatty alcohols and
wherein the hydrophobic liquid component is selected from the group consisting of plant oils, castor oil, jojoba oil, soybean oil, silicon oils, paraffin oils, and mineral oils, cremophor, oxethylated plant oils, oxethylated fatty alcohols, tocopherols, lipids, and phospholipids.

41. The pharmaceutical composition of claim 40, wherein the long-chain fatty acid is magnesium stearate.

42. The pharmaceutical composition of claim 40, wherein the long-chain alcohol is cetyl palmitate or cetyl alcohol.

43. The pharmaceutical composition of claim 22, wherein the effective amount of the lyophilized composition is between about 0.01 and about 50% (w/w).

44. The pharmaceutical composition of claim 43, wherein the effective amount of the lyophilized composition is about 0.2% (w/w).

45. The pharmaceutical composition according to claim 36, wherein the lyophilized composition is dispersed in the hydrophobic matrix in particulate form or in a dissolved state.

46. (canceled)

47. A dosage form comprising the pharmaceutical composition of claim 22, wherein the dosage form has a size and shape suitable for injection into a human or mammalian eye.

48. A method of treating ophthalmic disorders in a patient comprising administering an effective amount of the pharmaceutical composition of claim 22.

49. A method of treating ophthalmic disorders in a patient comprising administering an effective amount of the lyophilized composition of claim 1.

50. The method of claim 48, wherein the ophthalmic disorder is an inflammatory or degenerative ophthalmic disease effecting vascular function, neurological function or vascular and neurological function.

51. The method of claim 49, wherein the ophthalmic disorder is an inflammatory or degenerative ophthalmic disease effecting vascular function, neurological function or vascular and neurological function.

52. The method of claim 50, wherein the inflammatory or degenerative ophthalmic disease effecting vascular function, neurological function or vascular and neurological function is selected from the group consisting of wet and dry AMD, diabetic retinopathy, retinopathy of prematurity, punctate inner choroidopathy, retinal branch vein occlusion, iritis, uveitis, endophthalmitis, optic neuropathies, glaucoma, Stargardt's Disease, retinal detachment, Retinitis Pigmentosa, Juvenile retinoschisis, senile retinoschisis, limbal stem cell deficiency, corneal surface diseases, traumatic ocular injuries including injury to the cornea, traumatic brain injuries, traumatic ocular injuries, and traumatic injuries of the brain effecting vision or the retina.

53. The method of claim 51 , wherein the inflammatory or degenerative ophthalmic disease effecting vascular function, neurological function or vascular and neurological function is selected from the group consisting of wet and dry AMD, diabetic retinopathy, retinopathy of prematurity, punctate inner choroidopathy, retinal branch vein occlusion, iritis, uveitis, endophthalmitis, optic neuropathies, glaucoma, Stargardt's Disease, retinal detachment, Retinitis Pigmentosa, Juvenile retinoschisis, senile retinoschisis, limbal stem cell deficiency, corneal surface diseases, traumatic ocular injuries including injury to the cornea, traumatic brain injuries, traumatic ocular injuries, and traumatic injuries of the brain effecting vision or the retina.

54. The method of claim 48, wherein the pharmaceutical composition is administered at least every 2-6 months.

55. The method of claim 48, wherein the pharmaceutical composition is administered topically to the eye of the patient or by injection.

56. The method of claim 55, wherein the pharmaceutical composition is administered via intraocular injection.

57. The method of claim 56, wherein the pharmaceutical composition is injected into the vitreous chamber of the eye, injected sub-conjunctivally, injected intra-retinally, injected sub-tenon, or injected retrobulbar.

58. The method of claim 50, wherein regenerative factors released from the pharmaceutical composition decrease vascular permeability, decrease abnormal vascular growth, reduce damage to neurovascular tissue, reduce gliosis, improve or protect retinal function, improve or protect neurological function, improve or protect vision, or any combination thereof.

59. The method of claim 48, wherein the pharmaceutical composition comprises between 0.5 and 1 ml of the lyophilized composition and the sustained release drug delivery matrix.

60. The method of claim 59, wherein the pharmaceutical composition is micronized into a suspension prior to intravitreal injection.

61. The method of claim 48, wherein the effective amount of the lyophilized composition is between about 0.01 and about 50% (w/w).

62. The method of claim 61, wherein the effective amount of the lyophilized composition is about 0.2% (w/w).

63. A method of making the lyophilized composition of claim 1 comprising:
a) enzymatically digesting adipose tissue to obtain a population of adipose cells, wherein the population of adipose cells comprises at least one adipose stem cell (ASC);
b) culturing between adipose cells in a first culture medium at a seed density between 2 and 4×10⁵ cells/cm²;
c) passaging the cells in the first culture medium at least once;
d) selecting cells having at least 90% expression of one or more pericyte markers;
e) culturing the selected cells in a second culture medium, wherein the second culture medium is serum free and comprises at least one inflammatory cytokine;
f) transferring the selected cells into a basal culture medium that does not contain inflammatory cytokines;
g) removing cells from the basal culture medium to produce a cell-free conditioned medium comprising the secretome of the adipose cells; and
h) lyophilizing the conditioned medium.

64. The method of claim 63, wherein the adipose tissue is digested with collagenase.

65. The method of claim 63, wherein the one or more pericyte markers are selected from the group consisting of CD73, CD90, CD105, CD140b, and neural/glial antigen 2 (NG2).

66. The method of claim 63, wherein the at least one adipose stem cell is CD45⁻.

67. The method of claim 65, wherein at least 95% of the cells express the one or more pericyte markers.

68. The method of claim 63, wherein the inflammatory cytokine is selected from the group consisting of TNFα, IFNγ, and combinations thereof.

69. The method of claim 68, wherein the second serum free culture medium comprises between about 10 and about 30 ng/ml TNFα, between about 1 and about 20 ng/ml IFNγ, or a combination thereof.

70. The method of claim 69, wherein the second serum free culture medium comprises 20 ng/ml TNFα.

71. The method of claim 69, wherein the second serum free culture medium comprises 10 ng/ml IFNγ.

72. The method of claim 63, wherein culturing the cells in the second serum free culture medium containing at least one inflammatory cytokine increases TIMP1 expression by the cells.

73. The method of claim 63, wherein culturing the cells in the second serum free culture medium containing at least one inflammatory cytokine increases TSG-6 expression by the cells.

74. The method of claim 73, wherein TSG-6 expression is increased by at least 2 fold.

75. The method of claim 63, wherein culturing the cells in the presence of one or more inflammatory cytokines decreases the T cell activity of the lyophilized composition.

76. The method of claim 63, wherein culturing the cells in the second serum free culture medium containing at least one inflammatory cytokine increases the expression of one or more regenerative or anti-inflammatory factors by the cells.

77. The method of claim 76, wherein the one or more regenerative or anti-inflammatory factors is selected from the group consisting of Growth-regulated alpha protein (CXCL1), interleukin-6 (IL6), interleukin-8 (IL-8), C-C motif chemokine 2 (CCL2), C-C motif chemokine 8 (CCL8), C-C motif chemokine 5 (CCL5), C-X-C motif chemokine 10 (CXCL10), or Tumor necrosis factor receptor superfamily member 11B (TNFRSF11B), and any combination thereof.

78. The method of claim 75, wherein the cells are removed from the second serum free culture medium after 24 hours.

79. The method of claim 63, wherein the cells in the first culture medium are passaged 2, 3, 4, or 5 times.

80. The method of claim 63, wherein the conditioned media is stabilized by adding an effective amount of EDTA.

81. The method of claim 63, wherein the conditioned media is concentrated prior to lyophilization.

82. The method of claim 81, wherein the conditioned media is filtered using tangential flow filtration (TFF) at a molecular weight cut off (MWC) of about 5 kDa.

83. The method of claim 82, wherein, following TFF filtering, the conditioned media is diafiltered into Tris EDTA buffer or histidine buffer.

84. The method of claim 83, wherein, following diafiltration, an effective amount of sucrose is added as a lyophilization stabilizer.

85. A method of making the pharmaceutical composition of claim 22, comprising mixing an effective amount of the lyophilized composition with the sustained release drug delivery matrix to form a gel, paste-like, semi-solid drug composition, or microparticulate composition.

86. The method of claim 85, wherein the lyophilized composition is reconstituted in the sustained release drug delivery matrix.

87. The method of claim 85, wherein the pharmaceutical composition further comprises at least one excipient selected from the group consisting of monosaccharides, disaccharides, oligosaccharides, polysaccharides like hyaluronic acid, pectin, gum arabic and other gums, albumin, chitosan, collagen, collagen-n-hydroxysuccinimide, fibrin, fibrinogen, gelatin, globulin, polyaminoacids, polyurethane comprising amino acids, prolamin, protein-based polymers, copolymers and derivatives thereof, and mixtures thereof.

88. The method of claim 85, wherein forming of the gel, paste-like or semi-solid pharmaceutical composition includes repeated cycles of pressing and folding, in an algorithmic manner, of the mixture of the sustained release drug delivery matrix and the lyophilized composition.

89. The method for claim 85, further comprising:
forming the pharmaceutical composition into a suitable dosage form.

90. The method of claim 89, wherein the dosage form is suitable for intraocular injection.

91. The method of claim 49, wherein the lyophilized composition is administered at least every 2-6 months.

92. The method of claim 49, wherein the lyophilized composition is administered topically to the eye of the patient or by injection.

93. The method of claim 92, wherein the lyophilized composition is administered via intraocular injection.

94. The method of claim 93, wherein the lyophilized composition is injected into the vitreous chamber of the eye, injected sub-conjunctivally, injected intra-retinally, injected sub-tenon, or injected retrobulbar.

95. The method of claim 51, wherein regenerative factors released from the pharmaceutical composition decrease vascular permeability, decrease abnormal vascular growth, reduce damage to neurovascular tissue, reduce gliosis, improve or protect retinal function, improve or protect neurological function, improve or protect vision, or any combination thereof.
